(19) United States
(12) Patent Application Publication
(10) Pub. No.: US 2024/0317663 A1
(43) Pub. Date: Sep. 26, 2024

(54) VITAMIN D3-LIKE COMPOUND

(71) Applicant: KANAGAWA UNIVERSITY, Yokohama-shi, Kanagawa (JP)

(72) Inventors: Sentaro OKAMOTO, Yokohama-shi, Kanagawa (JP); Kota IBE, Yokohama-shi, Kanagawa (JP)

(21) Appl. No.: 18/573,553

(22) PCT Filed: Jun. 17, 2022
(86) PCT No.: PCT/JP2022/024408

§ 371 Date: Dec. 22, 2023

(30) Foreign Application Priority Data
Jun. 22, 2021 (WO) PCT/JP2021/023645

(51) Int. Cl. C07C 43/23; A61K 31/593; C07C 41/18

(52) U.S. Cl. CPC C07C 43/23; A61K 31/593; C07C 41/18

(57) ABSTRACT

Provided is a novel vitamin D3-like compound. A compound according to the present invention is represented by formula (1). In the formula, m and n are independently 0 or 1, p is an integer of 0-4, and s is an integer of 0-4. Y represents an oxygen atom, etc. R¹ represents a predetermined monovalent aliphatic hydrocarbon group. R² represents a predetermined alkoxy group, etc. R³ represents a predetermined divalent aliphatic hydrocarbon group which may have a substituent and may contain a heteroatom in a carbon chain. R⁴ represents a predetermined monovalent aliphatic hydrocarbon group. R⁵ represents a hydrogen atom, a predetermined alkoxy group which may have a substituent, etc. R⁶ represents a hydrogen atom or a monovalent aliphatic hydrocarbon group. R⁷ and R⁸ both represent a hydrogen atom or are integrated to represent a methylidene group.

### TECHNICAL FIELD

The present invention relates to a novel vitamin D3-like compound.

### BACKGROUND ART

Active vitamin D3 derivatives are used as therapeutic agents for osteoporosis as they act to promote calcium absorption in the small intestine and to regulate bone resorption and bone formation. They are also used for the treatment of secondary hyperparathyroidism with increased parathyroid hormone (PTH) as they have an inhibitory effect on the secretion of PTH. In addition to these activities, they have been found to have immunomodulating activity, cytostatic activity, and cell differentiation activity and have been studied on their use as therapeutic agents for, for example, cancer, psoriasis, rheumatoid arthritis, true diabetes, hypertension, acne, eczema, dermatitis, and other diseases.

In the conventional art, such active vitamin D3 derivatives useful for the treatment of various diseases are produced in a variety of forms (see, for example, Non-Patent Documents 1 and 2).

A known method for producing such vitamin D3 derivatives includes allowing vitamin D2 (starting material) of Formula (A) below to undergo cleavage at the sites indicated by the wavy lines to produce a common natural CD ring moiety; and subjecting the CD ring moiety to derivatization, such as formation of lower ring moieties or side chains.

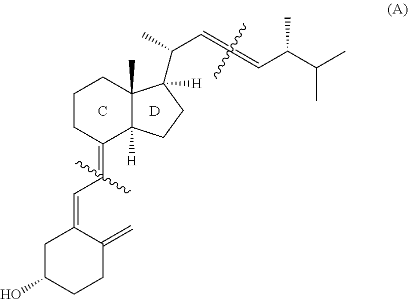

### CITATION LIST

### Non-Patent Document

Non-Patent Document 1: Maestro et al., J. Med. Chem., 2019, 62, 6854-6875
Non-Patent Document 2: Gogoi et al., J. Med. Chem., 2018, 61, 4928-4937

### DISCLOSURE OF THE INVENTION

### Problems to be Solved by the Invention

The CD ring moiety of the structure of Formula (A) above is difficult to synthesize chemically. Thus, vitamin D2 is often produced using cellular processes and then subjected to chemical synthesis processes. Unfortunately, the synthesis process using cells is costly, time-consuming, and laborious as compared to chemical synthesis. Therefore, the synthesis process using cells is disadvantageous in that it cannot easily produce a large amount of a wide variety of vitamin D3 derivatives.

It is an object of the present invention, which has been made in view of these circumstances, to provide a novel vitamin D3-like compound.

### Means for Solving the Problems

As a result of intensive studies for solving the problem described above, the inventors have created a compound of Formula (1) below and completed the present invention based on findings that the compound of Formula (1) acts as an agonist of the active vitamin D3 receptor. More specifically, the present invention provides the following aspects.

<1> A compound of Formula (1):

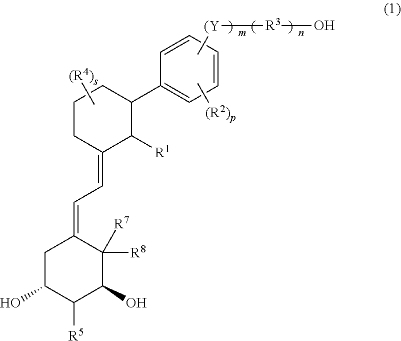

wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, R⁶ is a hydrogen atom or a monovalent aliphatic hydrocarbon group, and R⁷ and R⁸ are simultaneously hydrogen atoms or combined to form a methylidene group.

<2> A prophylactic or therapeutic agent for a disease responsive to active vitamin D₃, the prophylactic or therapeutic agent comprising the compound according to <1> as an active ingredient.

<3> The prophylactic or therapeutic agent according to <2>, wherein the disease responsive to active vitamin D₃ is osteoporosis, rickets, chronic hypocalcemia, renal osteodystrophy, secondary hyperparathyroidism, psoriasis, or cancer.

<4> A compound of Formula (2):

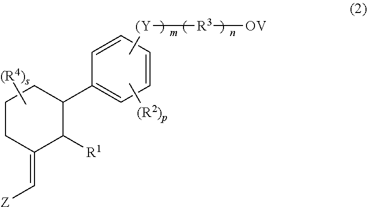

wherein m and n are independently 0 or 1, p is an integer of 0 to 4, s is an integer of 0 to 4, V is a hydrogen atom or a protecting group which is represented by W, Y is an oxygen atom, a sulfur atom, or a divalent group represented by —NR⁶—, Z is a halogen atom or a group represented by Formula (3) below, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, and R⁶ is a hydrogen atom or a monovalent aliphatic hydrocarbon group,

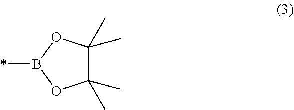 [SMILES: *B1OC(C)(C)C(C)(C)O1]

wherein * indicates a bond.

<5> A compound of Formula (4):

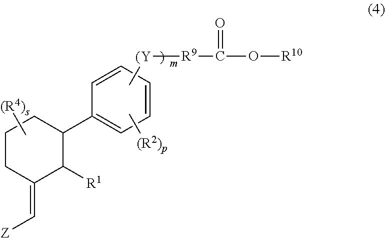

wherein m is 0 or 1, p is an integer of 0 to 4, s is an integer of 0 to 4, Y is an oxygen atom, a sulfur atom, or a divalent group represented by —NR⁶—, Z is a halogen atom or a group represented by Formula (3) below, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R⁴ is a linear or branched, saturated or unsaturated, monovalent aliphatic hydrocarbon group having 1 to 6 carbon atoms, R⁶ is a hydrogen atom or a monovalent aliphatic hydrocarbon group, R⁹ is a linear or branched, saturated or unsaturated, divalent aliphatic hydrocarbon group having 1 to 19 carbon atoms, optionally having a substituent, and optionally having a heteroatom-containing carbon chain, and R¹⁰ is an alkyl group having 1 to 8 carbon atoms,

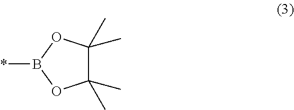 [SMILES: *B1OC(C)(C)C(C)(C)O1]

wherein * indicates a bond.

<6> A compound of Formula (5):

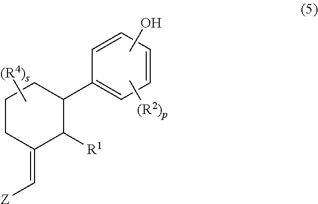

wherein p is an integer of 0 to 4, s is an integer of 0 to 6, Z is a halogen atom or a group represented by Formula (3) below, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, and R⁴ is a linear or branched, saturated or unsaturated, monovalent aliphatic hydrocarbon group having 1 to 6 carbon atoms,

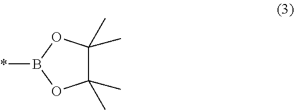 [SMILES: *B1OC(C)(C)C(C)(C)O1]

wherein * indicates a bond.

<7> A compound of Formula (6):

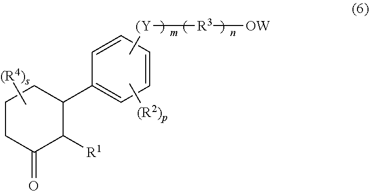

wherein m and n are independently 0 or 1, p is an integer of 0 to 4, s is an integer of 0 to 4, W is a protecting group, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, and R⁶ is a hydrogen atom or a monovalent aliphatic hydrocarbon group.

<8> A method for producing a compound of Formula (1) below, the method comprising:
allowing a compound of Formula (7) below to react with a compound of Formula (8) below to form a compound of Formula (9) below; and
removing groups represented by W from the compound of Formula (9) to form the compound of Formula (1),

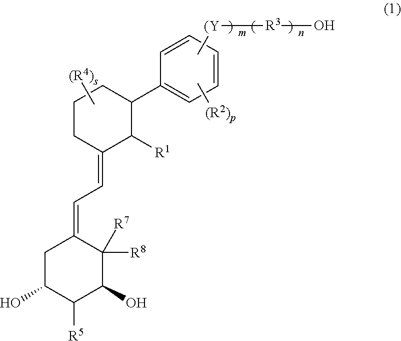

wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, R⁶ is a hydrogen atom or a monovalent aliphatic hydrocarbon group, and R⁷ and R⁸ are simultaneously hydrogen atoms or combined to form a methylidene group,

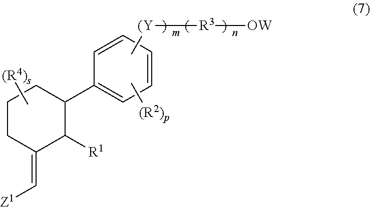

wherein m, n, p, s, Y, R¹, R², R³, R⁴, and R⁶ are as defined above, W is a protecting group, and Z¹ is a halogen atom or a group represented by Formula (3) below,

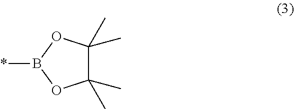 [SMILES: *B1OC(C)(C)C(C)(C)O1]

wherein * indicates a bond,

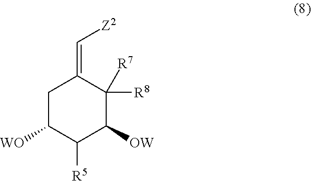

wherein R⁵, R⁷, R⁸, and W are as defined above, and Z² is a halogen atom or a group represented by Formula (3), provided that when Z¹ in Formula (7) is a halogen atom, Z² is a group represented by Formula (3) and that when Z¹ in Formula (7) is a group represented by Formula (3), Z² is a halogen atom,

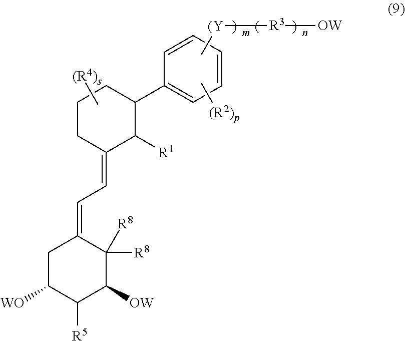

wherein m, n, p, s, W, Y, R¹, R², R³, R⁴, R⁵, R⁶, R⁷, and R⁸ are as defined above.

<9> A method for producing a compound of Formula (13) below, the method comprising:
allowing a compound of Formula (10) below to react with a compound of Formula (8) below to form a compound of Formula (11) below;
obtaining a compound of Formula (12) below from the compound of Formula (11); and
removing groups represented by W from the compound of Formula (12) to form the compound of Formula (13),

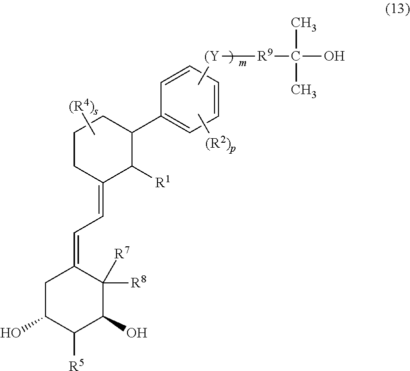

wherein m is 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, R⁶ is a hydrogen atom or a monovalent aliphatic hydrocarbon group, R⁷ and R⁸ are simultaneously hydrogen atoms or combined to form a methylidene group, and R⁹ is a linear or branched, saturated or unsaturated, divalent aliphatic hydrocarbon group having 1 to 19 carbon atoms, optionally having a substituent, and optionally having a heteroatom-containing carbon chain,

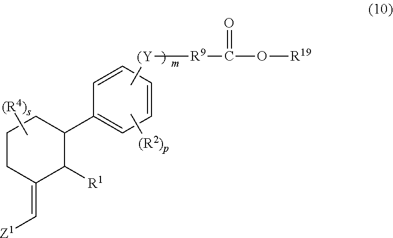

wherein m, p, s, Y, R¹, R², R⁴, R⁶, R⁹, and R¹⁰ are as defined above, R¹⁰ is an alkyl group having 1 to 8 carbon atoms, and Z¹ is a halogen atom or a group represented by Formula (3) below,

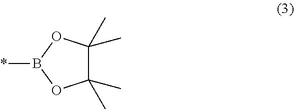 [SMILES: *B1OC(C)(C)C(C)(C)O1]

wherein * indicates a bond,

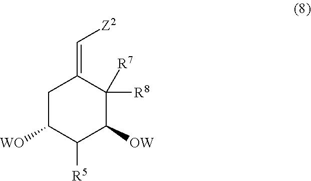

wherein R⁵, R⁷, and R⁸ are as defined above, W is a protecting group, and Z² is a halogen atom or a group represented by Formula (3), provided that when Z¹ in Formula (10) is a halogen atom, Z² is a group represented by Formula (3) and that when Z¹ in Formula (10) is a group represented by Formula (3), Z² is a halogen atom,

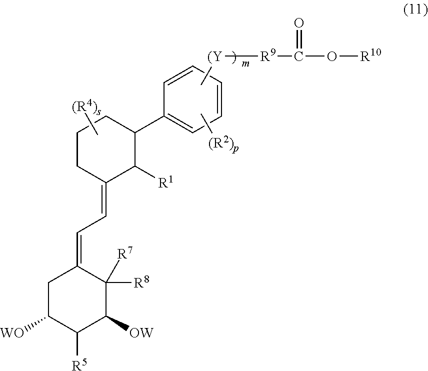

wherein m, p, s, W, Y, R¹, R², R⁴, R⁵, R⁶, R⁷, R⁸, R⁹, and R¹⁰ are as defined above,

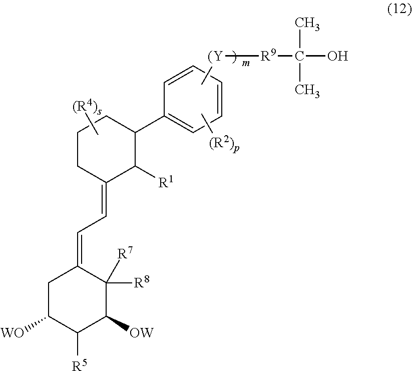

wherein m, p, s, W, Y, R¹, R², R⁴, R⁵, R⁶, R⁷, R⁸, and R⁹ are as defined above.

<10> A method for producing a compound of Formula (16) below, the method comprising:
allowing a compound of Formula (14) below to react with a compound of Formula (8) below to form a compound of Formula (15) below; and
removing groups represented by W from the compound of Formula (15) to form the compound of Formula (16),

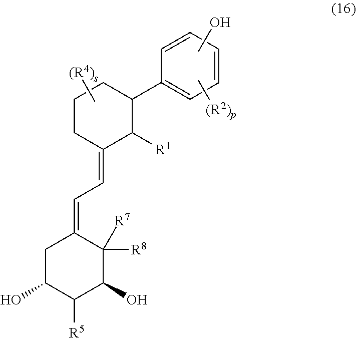

wherein p is an integer of 0 to 4, s is an integer of 0 to 4, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, and R⁷ and R⁸ are simultaneously hydrogen atoms or combined to form a methylidene group,

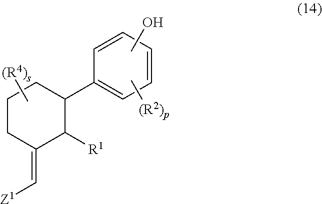

wherein p, s, R¹, R², and R⁴ are as defined above, and Z¹ is a halogen atom or a group represented by Formula (3) below,

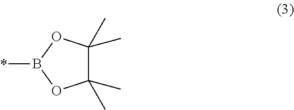 [SMILES: *B1OC(C)(C)C(C)(C)O1]

wherein * indicates a bond,

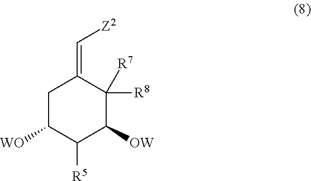

wherein R⁵, R⁷, and R⁸ are as defined above, W is a protecting group, and Z² is a halogen atom or a group represented by Formula (3), provided that when Z¹ in Formula (14) is a halogen atom, Z² is a group represented by Formula (3) and that when Z¹ in Formula (14) is a group represented by Formula (3), Z² is a halogen atom,

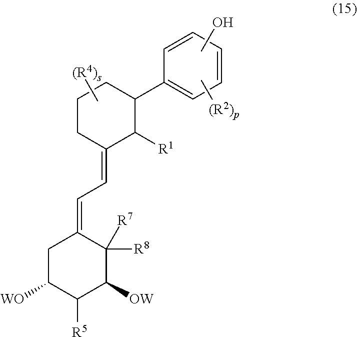

wherein p, s, W, R¹, R², R⁴, R⁵, R⁷, and R⁸ are as defined above.

<11> A method for producing a compound of Formula (19) below, the method comprising:
allowing a compound of Formula (6) below to react with a compound of Formula (17) below to form a compound of Formula (18) below; and
removing groups represented by W from the compound of Formula (18) to form the compound of Formula (19),

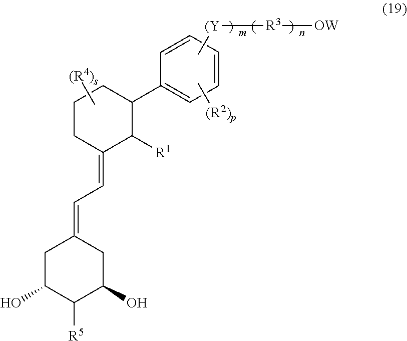

wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, and R⁶ is a hydrogen atom or a monovalent aliphatic hydrocarbon group,

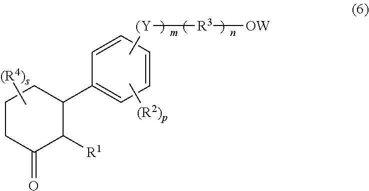

wherein m, n, p, s, Y, R¹, R², R³, R⁴, and R⁶ are as defined above, and W is a protecting group,

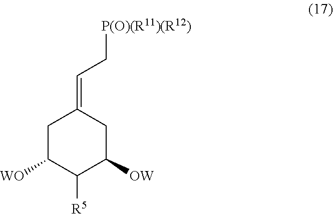

wherein R⁵ and W are as defined above, and R¹¹ and R¹² are independently a monovalent hydrocarbon group having 1 to 20 carbon atoms,

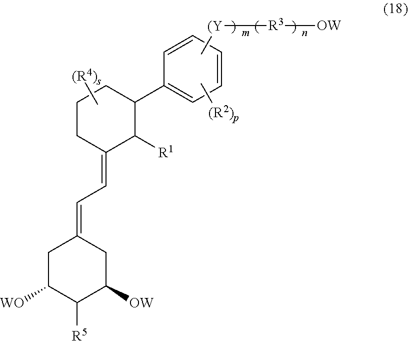

wherein m, n, p, s, W, Y, R¹, R², R³, R⁴, R⁵, and R⁶ are as defined above.

<12> A method for producing a compound of Formula (23) below, the method comprising:
allowing a compound of Formula (7) below to react with a compound of Formula (20) below to form a compound of Formula (21) below;
obtaining a compound of Formula (22) below from the compound of Formula (21); and
removing a group represented by W from the compound of Formula (22) to form the compound of Formula (23),

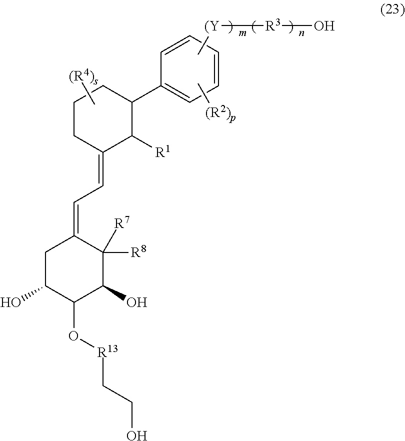

wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁶ is a hydrogen atom or a monovalent aliphatic hydrocarbon group, R⁷ and R⁸ are simultaneously hydrogen atoms or combined to form a methylidene group, and R¹³ is a linear or branched, saturated or unsaturated, divalent aliphatic hydrocarbon group having 1 to 4 carbon atoms, optionally having a substituent, and optionally having a heteroatom-containing carbon chain,

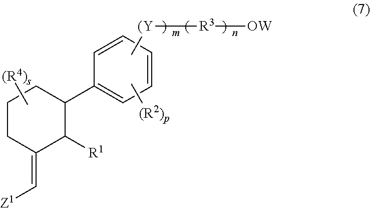

wherein m, n, p, s, Y, R¹, R², R³, R⁴, and R⁶ are as defined above, W is a protecting group, and Z¹ is a halogen atom or a group represented by Formula (3) below,

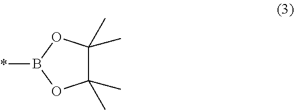 [SMILES: *B1OC(C)(C)C(C)(C)O1]

wherein * indicates a bond,

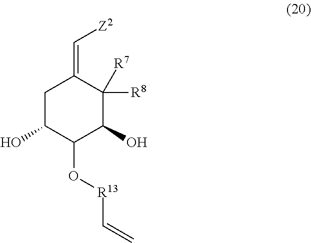

wherein R⁷, R⁸, and R¹³ are as defined above, and Z² is a halogen atom or a group represented by Formula (3), provided that when Z¹ in Formula (7) is a halogen atom, Z² is a group represented by Formula (3) and that when Z¹ in Formula (7) is a group represented by Formula (3), Z² is a halogen atom,

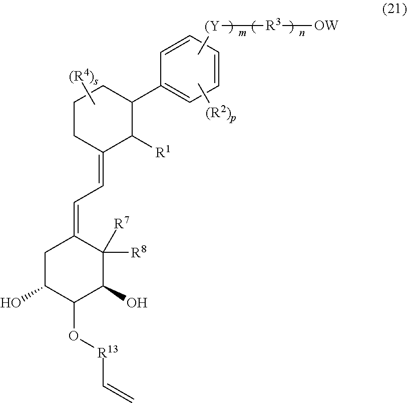

wherein m, n, p, s, Y, R¹, R², R³, R⁴, R⁶, R⁷, R⁸, and R¹³ are as defined above, and W is a protecting group,

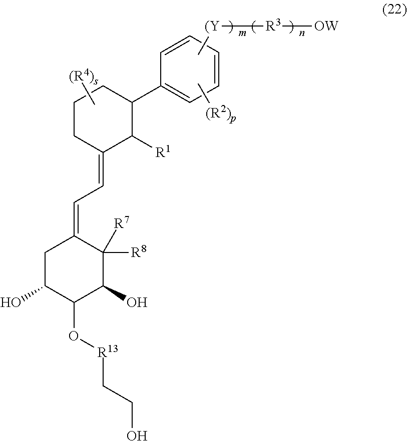

wherein m, n, p, s, Y, R¹, R², R³, R⁴, R⁶, R⁷, R⁸, R¹³, and W are as defined above.

<13> A method for producing a compound of Formula (27) below, the method comprising:
allowing a compound of Formula (6) below to react with a compound of Formula (24) below to form a compound of Formula (25) below;
removing groups represented by W from the compound of Formula (25) to form a compound of Formula (26) below; and
obtaining the compound of Formula (27) from the compound of Formula (26),

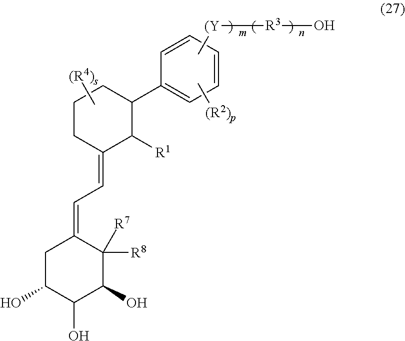

wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁶ is a hydrogen atom or a monovalent aliphatic hydrocarbon group, and R⁷ and R⁸ are simultaneously hydrogen atoms or combined to form a methylidene group,

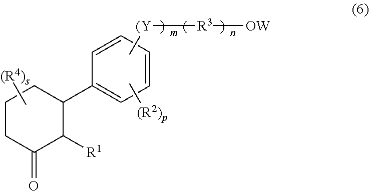

wherein m, n, p, s, Y, R¹, R², R³, R⁴, and R⁶ are as defined above, and W is a protecting group,

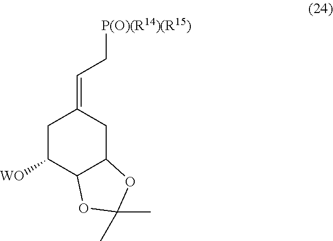

wherein W is as defined above, and R¹⁴ and R¹⁵ are independently a monovalent hydrocarbon group having 1 to 20 carbon atoms,

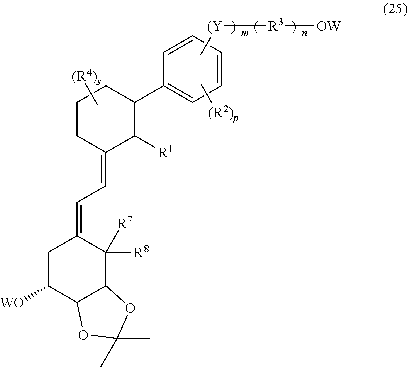

wherein m, n, p, s, Y, R¹, R², R³, R⁴, R⁶, R⁷, R⁸, and W are as defined above,

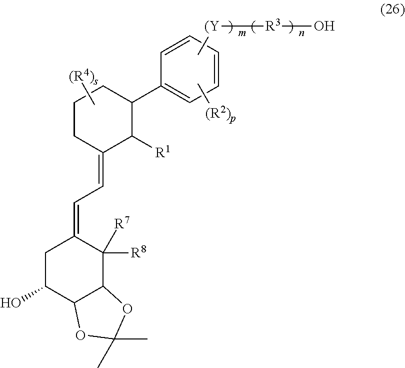

wherein m, n, p, s, Y, R¹, R², R³, R⁴, R⁶, R⁷, and R⁸ are as defined above.

### Effects of the Invention

The present invention provides a novel vitamin D3-like compound.

### BRIEF DESCRIPTION OF THE DRAWINGS

FIG. 1 is a graph showing concentration-activity curves obtained by a time-resolved fluorescence resonance energy transfer (TR-FRET) vitamin D receptor (VDR) coactivator assay performed in Test Example 1; and

FIG. 2 is a graph showing concentration-activity curves obtained by a vitamin D receptor (NR1I1, VDR) reporter assay performed in Test Example 2.

### PREFERRED MODE FOR CARRYING OUT THE INVENTION

Hereinafter, specific embodiments of the present invention will be described in detail. It should be noted that the embodiments described below are not intended to limit the present invention and may be altered or modified in various ways without departing from the gist of the present invention. In the description, the expression “X to Y”, in which X and Y are any numerical values for a certain range, means X or more and Y or less.

### Compound

An embodiment of the present invention is directed to a compound of Formula (1) below.

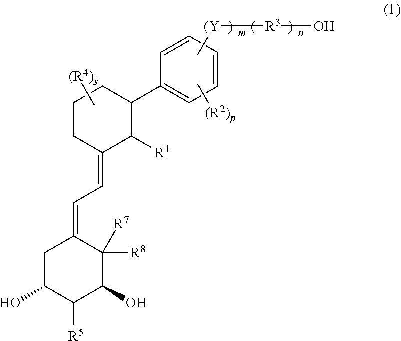

In Formula (1), m and n are independently 0 or 1. While m and n may be the same or different, m and n are preferably 1, or m is preferably 0 and n is preferably 1, or m and n are preferably 0, and m and n are more preferably 1, or m is more preferably 0 and n is more preferably 1.

In Formula (1), p is an integer of 0 to 4, and s is an integer of 0 to 4. When p is an integer of 2 or more, R²s may be the same or different. When s is an integer of 2 or more, R⁴s may be the same or different. In Formula (1), p and s are preferably independently an integer of 0 to 3, more preferably independently an integer of 0 to 2, even more preferably independently 0 or 1, furthermore preferably 0.

In Formula (1), Y is an oxygen atom, a sulfur atom, or a divalent group represented by —NR⁶—. In particular, Y is preferably an oxygen atom or a sulfur atom, more preferably an oxygen atom.

In Formula (1), R¹ is a linear or branched, saturated or unsaturated, monovalent aliphatic hydrocarbon group having 1 to 6 carbon atoms.

The linear or branched, saturated or unsaturated, monovalent aliphatic hydrocarbon group having 1 to 6 carbon atoms may be an alkyl group, such as methyl, ethyl, propyl, butyl, pentyl, hexyl, isopropyl, isobutyl, isopentyl, or tert-butyl; an alkenyl group, such as vinyl, allyl, or butenyl; an alkynyl group, such as ethynyl, propynyl, or butynyl; a cycloalkyl group, such as cyclopropyl, cyclobutyl, cyclopentyl, or cyclohexyl; or a cycloalkenyl group, such as cyclopentenyl or cyclohexenyl.

In particular, for steric structure, R¹ is preferably alkyl, more preferably methyl, ethyl, or propyl, even more preferably methyl or ethyl.

In Formula (1), R² is an alkyl group having 1 to 8 carbon atoms, a hydroxyl group, an alkoxy group having 1 to 8 carbon atoms, or a halogen atom.

The alkyl group having 1 to 8 carbon atoms may be methyl, ethyl, propyl, butyl, pentyl, hexyl, heptyl, or octyl. The alkoxy group having 1 to 8 carbon atoms may be methoxy, ethoxy, propoxy, isopropoxy, butoxy, pentoxy, hexoxy, or heptoxy. The halogen atom may be chlorine, bromine, iodine, or fluorine.

In particular, R² is preferably an alkyl group having 1 to 8 carbon atoms.

In Formula (1), R³ is a linear or branched, saturated or unsaturated, divalent aliphatic hydrocarbon group having 1 to 20 carbon atoms, optionally having a substituent, and optionally having a heteroatom-containing carbon chain.

The substituent may be a halogen atom, an epoxy group, a hydroxy group, or an amino group. The heteroatom may be oxygen, sulfur, or nitrogen. The linear or branched, saturated or unsaturated, divalent aliphatic hydrocarbon group having 1 to 20 carbon atoms may be methylene, ethylene, propylene, tetramethylene, ethylethylene, pentamethylene, or hexamethylene.

In particular, R³ is preferably —(CH₂)₂—, —(CH₂)₃—, —(CH₂)₄—, —CH₂—C(CH₃)₂—, —(CH₂)₂—C(CH₃)₂—, —(CH₂)₃—C(CH₃)₂—, —(CH₂)₄—C(CH₃)₂—, —CH₂—C(CH₂CH₃)₂—, —(CH₂)₂—C(CH₂CH₃)₂—, —(CH₂)₃—C(CH₂CH₃)₂—, —(CH₂)₄—C(CH₂CH₃)₂—, —C≡C—CH₂—C(CH₃)₂—, —C≡C—CH₂—C(CH₂CH₃)₂—, or —C≡C—CH₂—C(CH₂CH₂CH₃)₂—, more preferably —CH₂—C(CH₃)₂—, —(CH₂)₂—C(CH₃)₂—, —(CH₂)₃—C(CH₃)₂—, —(CH₂)₄—C(CH₃)₂—, —C≡C—CH₂—C(CH₂CH₃)₂—, or —C≡C—CH₂—C(CH₂CH₂CH₃)₂—, even more preferably —CH₂—C(CH₃)₂—, —(CH₂)₂—C(CH₃)₂—, —(CH₂)₃—C(CH₃)₂—, —C≡C—CH₂—C(CH₂CH₃)₂—, or —C≡C—CH₂—C(CH₂CH₂CH₃)₂—.

In Formula (1), R⁴ is a linear or branched, saturated or unsaturated, monovalent aliphatic hydrocarbon group having 1 to 6 carbon atoms.

The linear or branched, saturated or unsaturated, monovalent aliphatic hydrocarbon group having 1 to 6 carbon atoms may be an alkyl group, such as methyl, ethyl, propyl, butyl, pentyl, hexyl, isopropyl, isobutyl, isopentyl, or tert-butyl; an alkenyl group, such as vinyl, allyl, or butenyl; an alkynyl group, such as ethynyl, propynyl, or butynyl; a cycloalkyl group, such as cyclopropyl, cyclobutyl, cyclopentyl, or cyclohexyl; or a cycloalkenyl group, such as cyclopentenyl or cyclohexenyl.

In particular, R⁴ is preferably alkyl, more preferably isopropyl.

In Formula (1), R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent.

The substituent may be a halogen atom, an epoxy group, a hydroxy group, or an amino group. The alkoxy group having 1 to 6 carbon atoms may be methoxy, ethoxy, propoxy, isopropoxy, butoxy, pentoxy, or hexoxy. The linear or branched, saturated or unsaturated, monovalent aliphatic hydrocarbon group having 1 to 6 carbon atoms may be an alkyl group, such as methyl, ethyl, propyl, butyl, pentyl, hexyl, isopropyl, isobutyl, isopentyl, or tert-butyl; an alkenyl group, such as vinyl, allyl, or butenyl; an alkynyl group, such as ethynyl, propynyl, or butynyl; a cycloalkyl group, such as cyclopropyl, cyclobutyl, cyclopentyl, or cyclohexyl; or a cycloalkenyl group, such as cyclopentenyl or cyclohexenyl.

In particular, R⁵ is preferably a hydrogen atom or an alkoxy group having 1 to 6 carbon atoms and optionally having a substituent, more preferably a hydrogen atom.

In Formula (1), R⁶ is a hydrogen atom or a monovalent aliphatic hydrocarbon group.

The monovalent aliphatic hydrocarbon group may be, for example, a linear or branched, saturated or unsaturated, aliphatic hydrocarbon group having 1 to 6 carbon atoms. Specifically, the monovalent aliphatic hydrocarbon group may be an alkyl group, such as methyl, ethyl, propyl, butyl, pentyl, hexyl, isopropyl, isobutyl, isopentyl, or tert-butyl; an alkenyl group, such as vinyl, allyl, or butenyl; an alkynyl group, such as ethynyl, propynyl, or butynyl; a cycloalkyl group, such as cyclopropyl, cyclobutyl, cyclopentyl, or cyclohexyl; or a cycloalkenyl group, such as cyclopentenyl or cyclohexenyl.

In particular, R⁶ is preferably a hydrogen atom or an alkyl group.

In Formula (1), R⁷ and R⁸ are simultaneously hydrogen atoms or combined to form a methylidene group.

Some examples of the compound of Formula (1) are shown below. It should be noted that these examples are non-limiting examples of the compound of Formula (1).

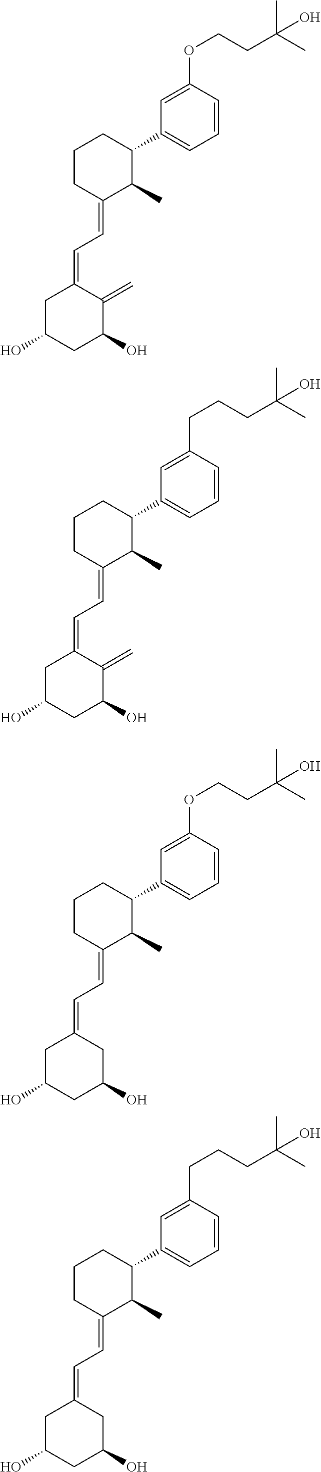 [SMILES: C=C1/C(=C\C=C2/CCC[C@H](c3cccc(OCCC(C)(C)O)c3)[C@H]2C)C[C@@H](O)C[C@@H]1O ; C=C1/C(=C\C=C2/CCC[C@H](c3cccc(CCCC(C)(C)O)c3)[C@H]2C)C[C@@H](O)C[C@@H]1O ; C[C@H]1/C(=C/C=C2C[C@@H](O)C[C@H](O)C2)CCC[C@@H]1c1cccc(OCCC(C)(C)O)c1 ; C[C@H]1/C(=C/C=C2C[C@@H](O)C[C@H](O)C2)CCC[C@@H]1c1cccc(CCCC(C)(C)O)c1]

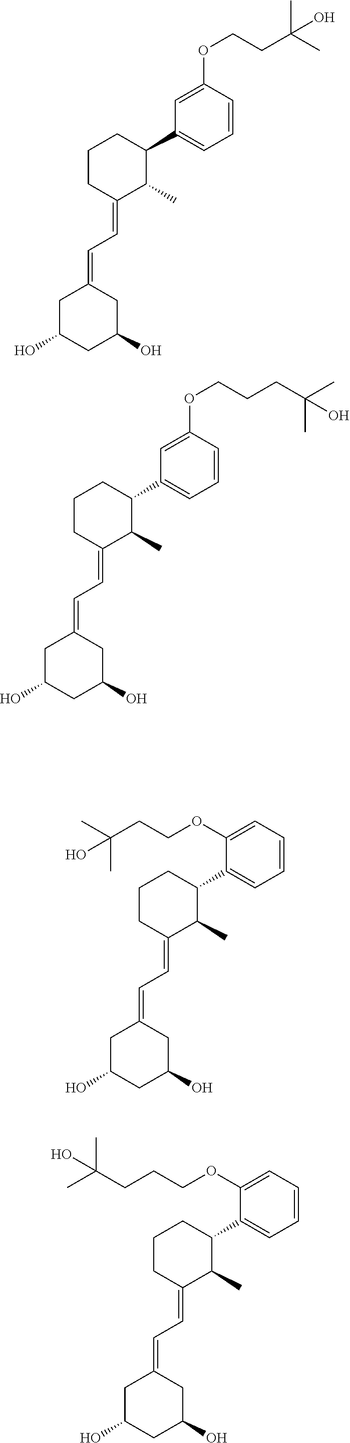 [SMILES: C[C@@H]1/C(=C/C=C2C[C@@H](O)C[C@H](O)C2)CCC[C@H]1c1cccc(OCCC(C)(C)O)c1 ; C[C@H]1/C(=C/C=C2C[C@@H](O)C[C@H](O)C2)CCC[C@@H]1c1cccc(OCCCC(C)(C)O)c1 ; C[C@H]1/C(=C/C=C2C[C@@H](O)C[C@H](O)C2)CCC[C@@H]1c1ccccc1OCCC(C)(C)O ; C[C@H]1/C(=C/C=C2C[C@@H](O)C[C@H](O)C2)CCC[C@@H]1c1ccccc1OCCCC(C)(C)O]

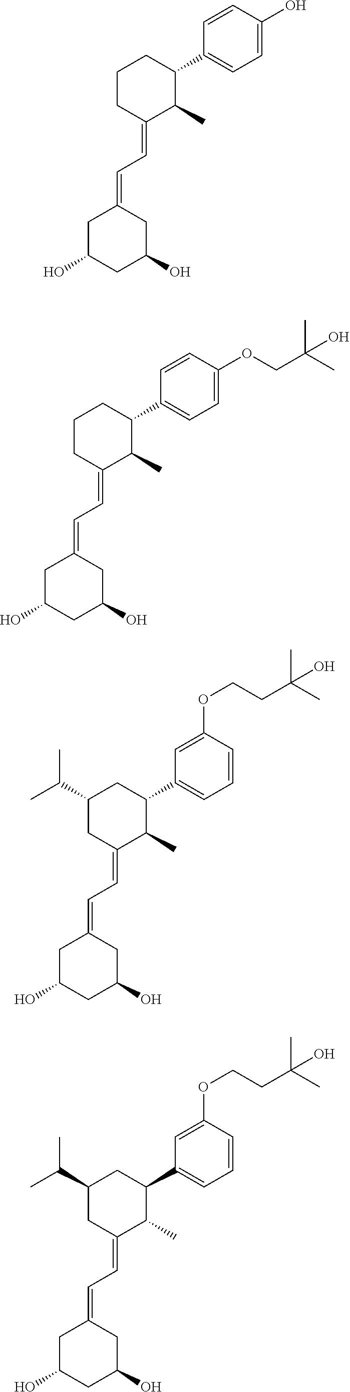 [SMILES: C[C@H]1/C(=C/C=C2C[C@@H](O)C[C@H](O)C2)CCC[C@@H]1c1ccc(O)cc1 ; C[C@H]1/C(=C/C=C2C[C@@H](O)C[C@H](O)C2)CCC[C@@H]1c1ccc(OCC(C)(C)O)cc1 ; CC(C)[C@H]1C/C(=C\C=C2C[C@@H](O)C[C@H](O)C2)[C@H](C)[C@@H](c2cccc(OCCC(C)(C)O)c2)C1 ; CC(C)[C@@H]1C/C(=C\C=C2C[C@@H](O)C[C@H](O)C2)[C@@H](C)[C@H](c2cccc(OCCC(C)(C)O)c2)C1]

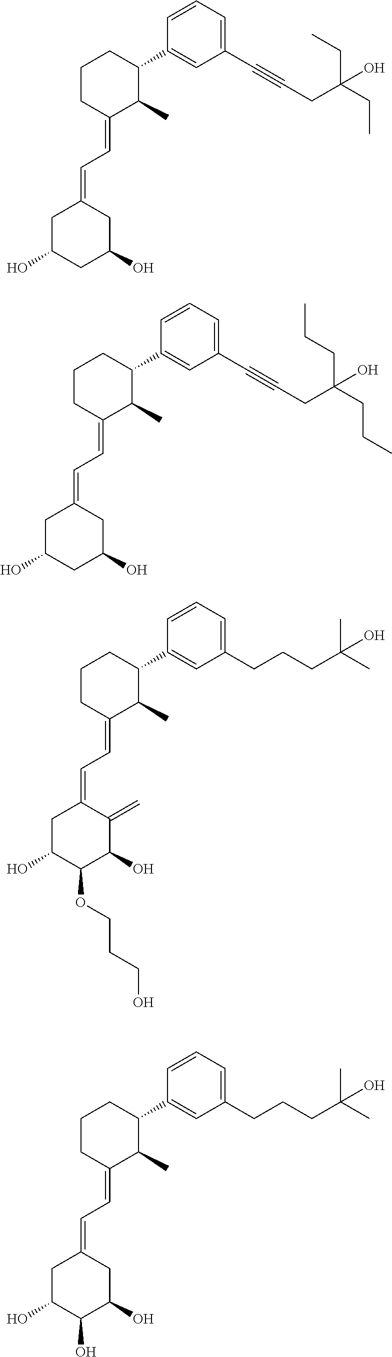

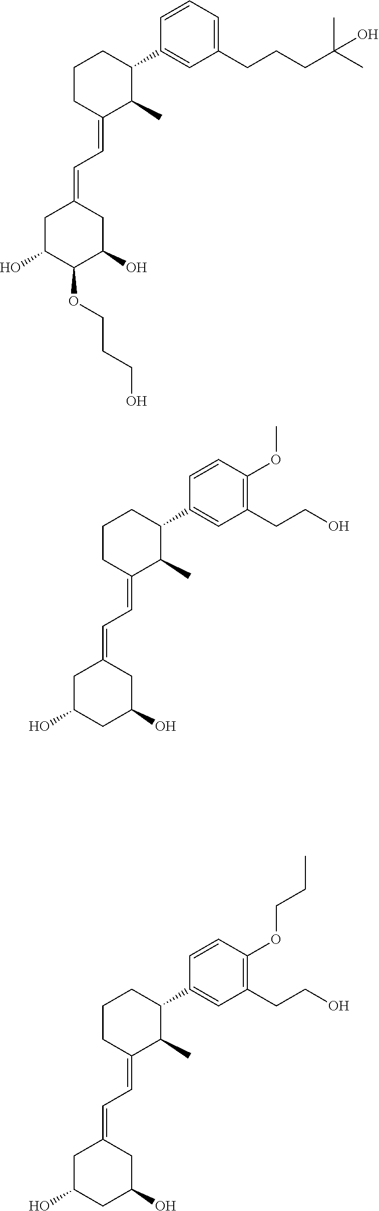

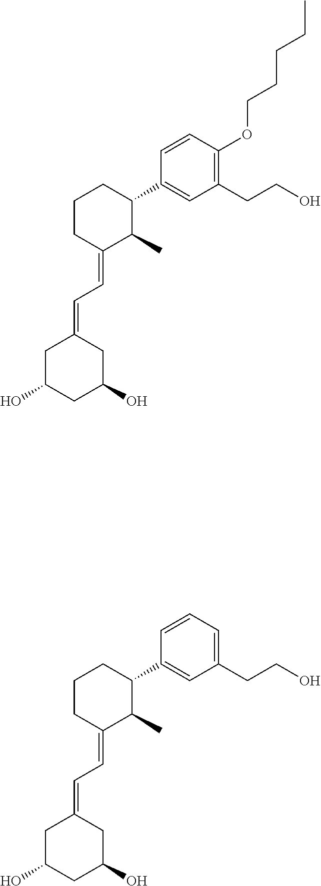 [SMILES: CCCCCOc1ccc([C@H]2CCC/C(=C\C=C3C[C@@H](O)C[C@H](O)C3)[C@@H]2C)cc1CCO ; C[C@H]1/C(=C/C=C2C[C@@H](O)C[C@H](O)C2)CCC[C@@H]1c1cccc(CCO)c1]

The compound of Formula (1) may have an acidic or basic functional group. In that case, the compound of Formula (1) may be in the form of a pharmaceutically acceptable salt. For example, the compound of Formula (1) having an acidic functional group may be in the form of an alkali metal salt (e.g., a sodium salt, a potassium salt), an alkaline-earth metal salt (e.g., a calcium salt, a magnesium salt), or an ammonium salt. For example, the compound of Formula (1) having a basic functional group may be in the form of a salt with an inorganic acid, such as hydrochloric acid, hydrobromic acid, nitric acid, sulfuric acid, or phosphoric acid, or in the form of a salt with an organic acid, such as acetic acid, phthalic acid, fumaric acid, oxalic acid, tartaric acid, maleic acid, citric acid, succinic acid, methanesulfonic acid, benzenesulfonic acid, or p-toluenesulfonic acid.

Prophylactic or Therapeutic Agent There is provided a prophylactic or therapeutic agent including the compound of Formula (1) as an active ingredient. Such a prophylactic or therapeutic agent is effective against diseases responsive to active vitamin D3. The compound of Formula (1) is as described above, and a detailed description thereof will not be repeated here. The term “prophylactic” is intended to include not only preventing the onset of diseases but also delaying the onset of diseases. The term “therapeutic” is intended to include not only eliminating or reducing the symptoms of diseases but also controlling the rate of progression of symptoms.

Examples of diseases responsive to active vitamin D3 include osteoporosis, rickets, chronic hypocalcemia, renal osteodystrophy, secondary hyperparathyroidism, psoriasis, and cancer.

The prophylactic or therapeutic agent may be produced by any suitable method employed in the field of pharmaceuticals or by any suitable modification thereof.

The prophylactic or therapeutic agent may contain an additional ingredient in addition to the compound of Formula (1). For example, the prophylactic or therapeutic agent may contain an organic or inorganic carrier commonly used as a formulation material. To form a solid formulation, the carrier may be used as an excipient, a lubricant, a binder, or a disintegrant. To form a liquid formulation, the carrier may be used as a solvent, a dissolution aid, a suspending agent, an isotonic agent, or a buffering agent. The prophylactic or therapeutic agent may also contain a formulation additive, such as a preservative, an antioxidant, a coloring agent, or a sweetening agent.

The prophylactic or therapeutic agent may be in any dosage form. Dosage forms of the prophylactic or therapeutic agent include oral dosage forms, such as tablets, capsules, granules, dispersions, troches, syrups, emulsions, suspensions, and films; and parenteral dosage forms, such as injections, infusions, topical agents, suppositories, pellets, nasal agents, transpulmonary agents (inhalants), and eye drops.

The prophylactic or therapeutic agent may be administered to any subject. Preferably, the prophylactic or therapeutic agent is administered to mammals. Mammals include both humans and non-human animals (e.g., mice, rats, hamsters, rabbits, cats, dogs, cows, sheep, monkeys).

The dosage of the prophylactic or therapeutic agent may be appropriately determined according to the administration subject, route of administration, target disease or symptom, or other factors.

The prophylactic or therapeutic agent may be administered in combination with any other drug depending on the purpose of administration or other purposes. The type, dosage, and other characteristics of the drug to be used in combination with the prophylactic or therapeutic agent may be appropriately selected based on the desired effect and other factors. The concomitant drug and the prophylactic or therapeutic agent may be administered simultaneously or separately.

### Intermediate Compounds

A compound of Formula (2) below is provided as an intermediate compound to form the compound of Formula (1).

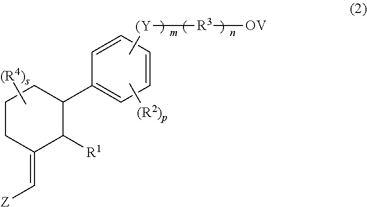

In Formula (2), m, n, p, s, Y, R¹, R², R³, R⁴, and R⁶ are as defined above.

In Formula (2), V is a hydrogen atom or a protecting group which is represented by W. The protecting group may be a silyl ether protecting group, such as TMS (trimethylsilyl), TES (triethylsilyl), or TBS (tert-butyldimethylsilyl).

In Formula (2), Z is a halogen atom or a group represented by Formula (3) below. The halogen atom may be chlorine, bromine, iodine, or fluorine.

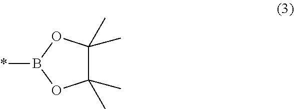 [SMILES: *B1OC(C)(C)C(C)(C)O1]

In Formula (3), * indicates a bond.

A compound of Formula (4) below is also provided as an intermediate compound to form the compound of Formula (1).

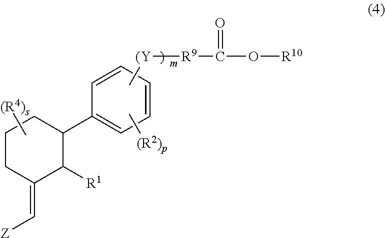

In Formula (4), m, p, s, Y, Z, R¹, R², R⁴, and R⁶ are as defined above.

In Formula (4), R⁹ is a linear or branched, saturated or unsaturated, divalent aliphatic hydrocarbon group having 1 to 19 carbon atoms, optionally having a substituent, and optionally having a heteroatom-containing carbon chain.

The heteroatom may be oxygen, sulfur, or nitrogen. The linear or branched, saturated or unsaturated, divalent aliphatic hydrocarbon group having 1 to 19 carbon atoms may be methylene, ethylene, propylene, tetramethylene, ethylethylene, pentamethylene, or hexamethylene.

In Formula (4), R¹⁰ is an alkyl group having 1 to 8 carbon atoms. The alkyl group having 1 to 8 carbon atoms may be methyl, ethyl, propyl, butyl, pentyl, hexyl, heptyl, or octyl.

A compound of Formula (5) below is also provided as an intermediate compound to form the compound of Formula (1).

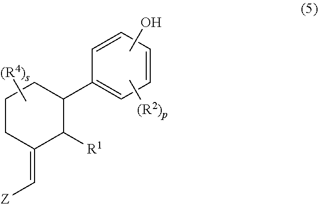

In Formula (5), p, s, Z, R¹, R², and R⁴ are as defined above.

A compound of Formula (6) below is also provided as an intermediate compound to form the compound of Formula (1).

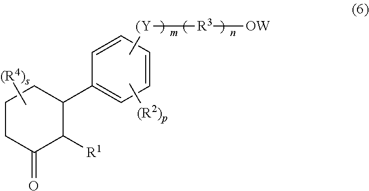

In Formula (6), m, n, p, s, W, Y, R¹, R², R³, R⁴, and R⁶ are as defined above.

### Methods for Producing Intermediate Compounds

A method for producing the intermediate compound of Formula (2), (4), (5), or (6) above will be described with reference to Scheme 1 below. In the description, “OMOM”, “i-Bu”, and “Tf” represent a methoxymethoxy group, an isobutyl group, and a trifluoromethylsulfonyl group, respectively.

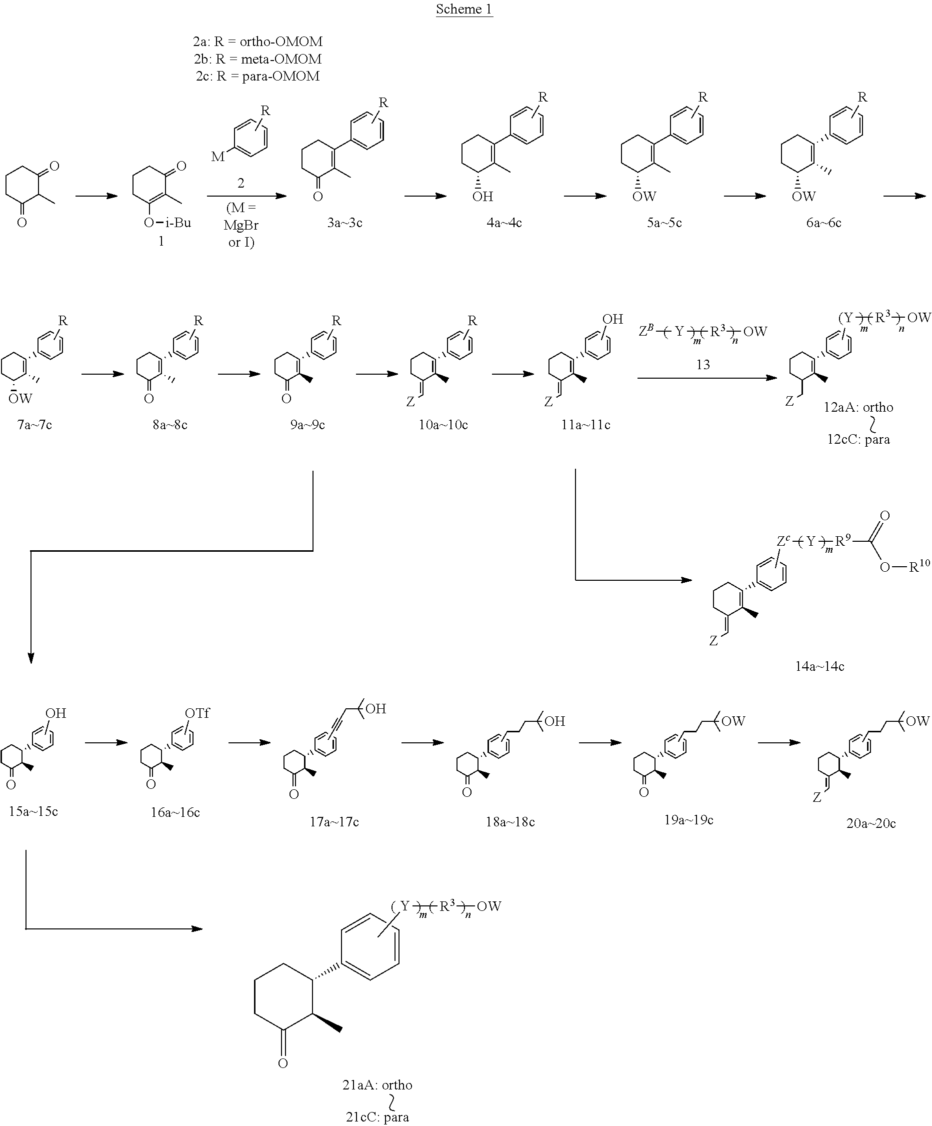

Compound 1 is allowed to react with compound 2 to form compounds 3a-3c.

Compounds 3a-3c are allowed to react with, for example, a solution of (S)-α,α-diphenylprolinol, trimethyl borate, and N,N-diethylaniline borane in tetrahydrofuran to form compounds 4a-4c.

Z^{A}—W and compounds 4a-4c are allowed to react to form compounds 5a-5c, in which Z^{A} is a halogen atom, which may be chlorine, bromine, iodine, or fluorine and is preferably chlorine, and the protecting group W may be as described above.

Compounds 5a-5c are allowed to react under a hydrogen atmosphere to form compounds 6a-6c. This reaction may be carried out using, for example, palladium as a catalyst.

The protecting group W is removed from compounds 6a-6c to form compounds 7a-7c.

Compounds 7a-7c are allowed to react with an oxidizing agent to form compounds 8a-8c. The oxidizing agent may be, for example, pyridinium chlorochromate.

Compounds 8a-8c are isomerized to form compounds 9a-9c.

Compounds 9a-9c are allowed to react with a halogenating agent to form compounds 10a-10c. The halogenating agent may be, for example, (bromomethyl)triphenylphosphonium bromide.

Compounds 10a-10c are allowed to react with an acid to form compounds 11a-11c. The acid may be, for example, hydrochloric acid.

Compounds 11a-11c are allowed to react with compound 13 to form compounds 12aA-12cC. In the formula of compound 13, m, n, W, Y, and R³ are as defined above. In the formula of compound 13, Z^{B} is a halogen atom, which may be chlorine, bromine, iodine, or fluorine.

Compounds 11a-11c are allowed to react with a compound of Formula (B) below to form compounds 14a-14c. In Formula (B) below, Zc is a halogen atom, which may be chlorine, bromine, iodine, or fluorine.

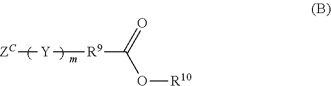

Compounds 9a-9c are allowed to react with an acid to form compounds 15a-15c. The acid may be, for example, hydrochloric acid.

Compounds 15a-15c are allowed to react with trifluoromethanesulfonic anhydride to form compounds 16a-16c.

Compounds 16a-16c are allowed to react with 2-methylpent-4-yn-2-ol to form compounds 17a-17c.

Compounds 17a-17c are allowed to react under a hydrogen atmosphere to form compounds 18a-18c. This reaction may be carried out using, for example, palladium as a catalyst.

Z^{D}—W and compounds 18a-18c are allowed to react to form compounds 19a-19c, in which Z^{D} is a halogen atom, which may be chlorine, bromine, iodine, or fluorine and is preferably chlorine, and the protecting group W may be as described above.

Compounds 19a-19c are allowed to react with a halogenating agent to form compounds 20a-20c. The halogenating agent may be, for example, (bromomethyl)triphenylphosphonium bromide.

Compounds 15a-15c are allowed to react with compound 13 to form compounds 21aA-21cC. In the formula of compound 13, m, n, W, Y, Z^{B}, and R³ are as defined above.

Next, a method for producing the intermediate will be described with reference to Scheme 2 below.

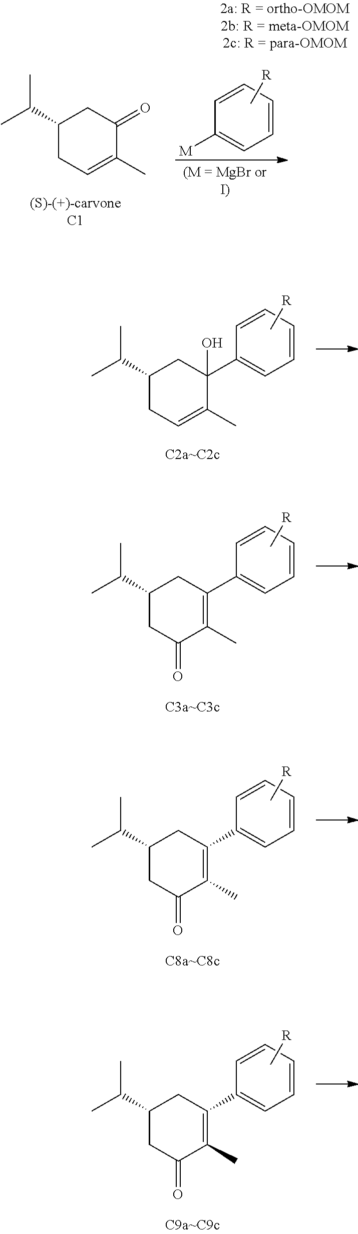

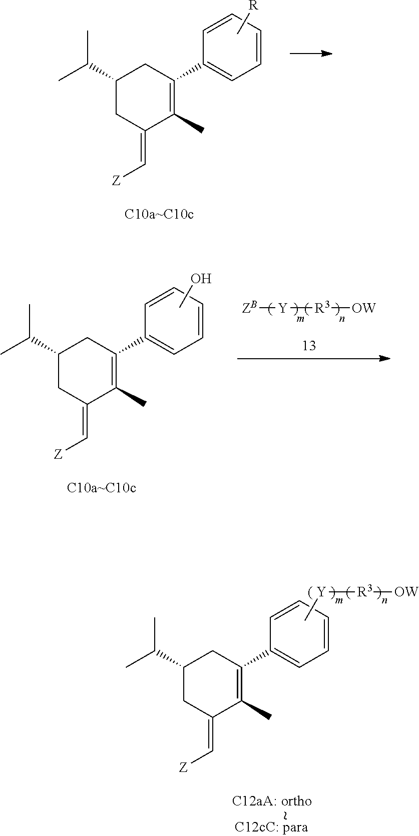

Compound C1 is allowed to react with compound 2 to form compounds C2a-C2c.

Compounds C2a-C2c are allowed to react with an oxidizing agent to form compounds C3a-C3c. The oxidizing agent may be, for example, pyridinium chlorochromate.

Compounds C3a-C3c are allowed to react under a hydrogen atmosphere to form compounds C8a-C8c. This reaction may be carried out using, for example, palladium as a catalyst.

Compounds C8a-C8c are isomerized to form compounds C9a-C9c.

Compounds C9a-C9c are allowed to react with a halogenating agent to form compounds C10a-C10c. The halogenating agent may be, for example, (bromomethyl)triphenylphosphonium bromide.

Compounds C10a-C10c are allowed to react with an acid to form compounds C11a-C11c. The acid may be, for example, hydrochloric acid.

Compounds C11a-C11c are allowed to react with compound 13 to form compounds C12aA-C12cC. In the formula of compound 13, m, n, Y, W, Z^{B}, and R³ are as defined above.

### Method for Producing Final Compound

A method for producing the compound of Formula (1) includes allowing a compound of Formula (7) below to react with a compound of Formula (8) below to form a compound of Formula (9) below; and removing the groups represented by W from the compound of Formula (9) to form the compound of Formula (1).

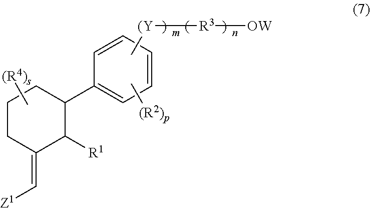

In Formula (7), m, n, p, s, W, Y, R¹, R², R³, R⁴, and R⁶ are as defined above. Z¹ is a halogen atom or a group represented by Formula (3) below. The halogen atom may be chlorine, bromine, iodine, or fluorine.

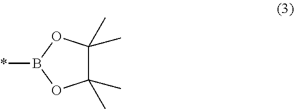 [SMILES: *B1OC(C)(C)C(C)(C)O1]

In Formula (3), * indicates a bond.

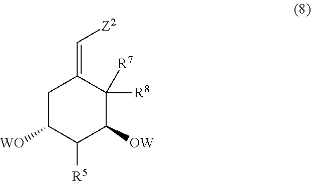

In Formula (8), R⁵, R⁷, R⁸, and W are as defined above.

In Formula (8), Z² is a halogen atom or a group represented by Formula (3) above. The halogen atom may be chlorine, bromine, iodine, or fluorine. In Formula (7), when Z¹ is a halogen atom, Z² is a group represented by Formula (3) above, and when Z¹ is a group represented by Formula (3) above, Z² is a halogen atom.

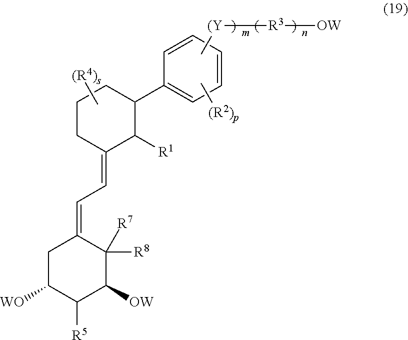

In Formula (9), m, n, p, s, W, Y, R¹, R², R³, R⁴, R⁵, R⁶, R⁷, and R⁸ are as defined above.

A method for producing a compound of Formula (13) below includes allowing a compound of Formula (10) below to react with a compound of Formula (8) below to form a compound of Formula (11) below; obtaining a compound of Formula (12) below from the compound of Formula (11); and removing the groups represented by W from the compound of Formula (12) to form the compound of Formula (13).

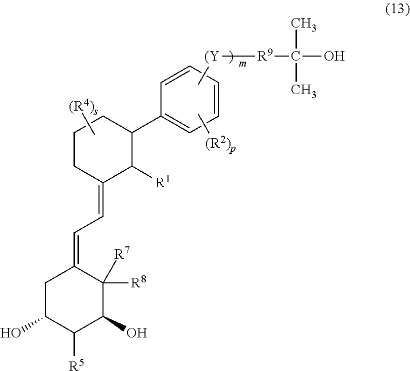

In Formula (13), m, p, s, Y, R¹, R², R⁴, R⁵, R⁶, R⁷, R⁸, and R⁹ are as defined above.

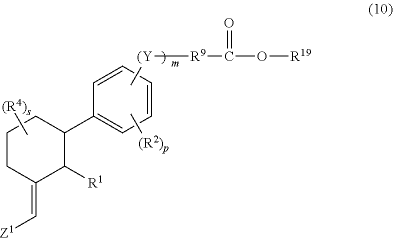

In Formula (10), m, p, s, Y, R¹, R², R⁴, R⁶, R⁹, R¹⁰, and Z¹ are as defined above.

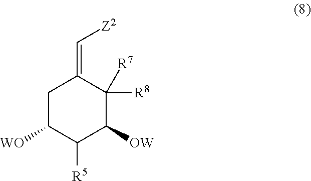

In Formula (8), W, Z², R⁵, R⁷, and R⁸ are as defined above.

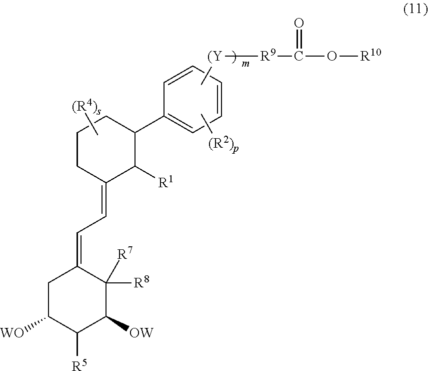

In Formula (11), m, p, s, W, Y, R¹, R², R⁴, R⁵, R⁶, R⁷, R⁸, R⁹, and R¹⁰ are as defined above.

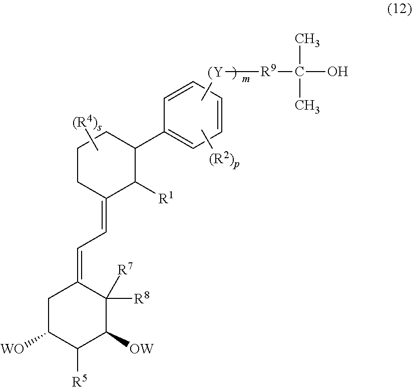

In Formula (12), m, p, s, W, Y, R¹, R², R⁴, R⁵, R⁶, R⁷, R⁸, and R⁹ are as defined above.

A method for producing a compound of Formula (16) below includes allowing a compound of Formula (14) below to react with a compound of Formula (8) below to form a compound of Formula (15) below; and removing the groups represented by W from the compound of Formula (15) to form the compound of Formula (16).

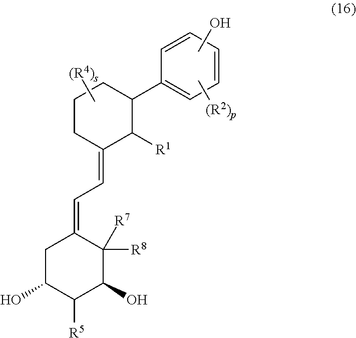

In Formula (16), p, s, R¹, R², R⁴, R⁵, R⁷, and R⁸ are as defined above.

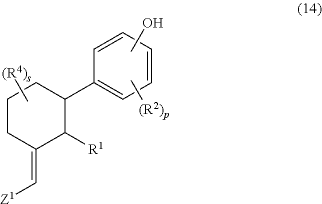

In formula (14), p, s, Z¹, R¹, R², and R⁴ are as defined above.

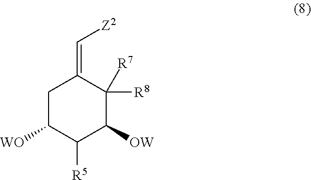

In Formula (8), W, Z², R⁵, R⁷, and R⁸ are as defined above.

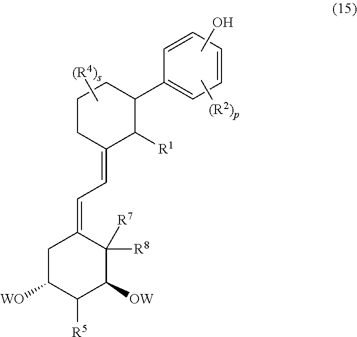

In Formula (15), p, s, W, R¹, R², R⁴, R⁵, R⁷, and R⁸ are as defined above.

A method for producing a compound of Formula (19) below includes allowing a compound of Formula (6) below to react with a compound of Formula (17) below to form a compound of Formula (18) below; and removing the groups represented by W from the compound of Formula (18) to form the compound of Formula (19).

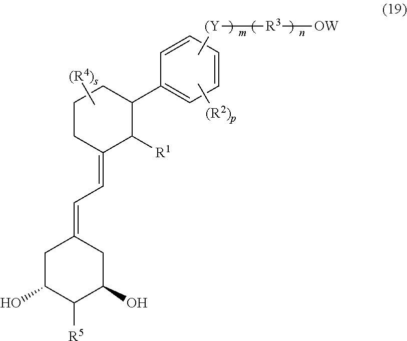

In Formula (19), m, n, p, s, Y, R¹, R², R³, R⁴, R⁵, and R⁶ are as defined above.

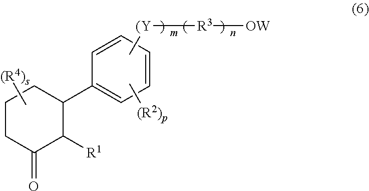

In Formula (6), m, n, p, s, W, Y, R¹, R², R³, R⁴, and R⁶ are as defined above.

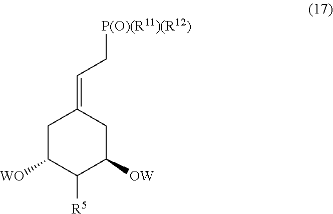

In Formula (17), R⁵ and W are as defined above. R¹¹ and R¹² are independently a monovalent hydrocarbon group having 1 to 20 carbon atoms. The monovalent hydrocarbon group having 1 to 20 carbon atoms may be an aryl group having 6 to 20 carbon atoms, such as phenyl or naphthyl.

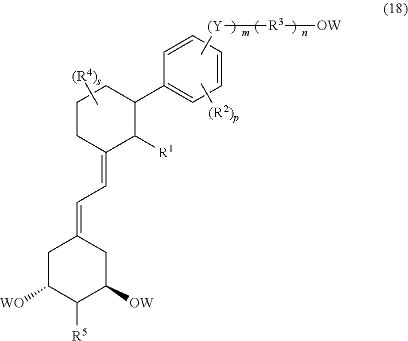

In Formula (18), m, n, p, s, W, Y, R¹, R², R³, R⁴, R⁵, and R⁶ are as defined above.

A method for producing a compound of Formula (23) below includes allowing a compound of Formula (7) below to react with a compound of Formula (20) below to form a compound of Formula (21) below; obtaining a compound of Formula (22) below from the compound of Formula (21); and removing the group represented by W from the compound of Formula (22) to form the compound of Formula (23).

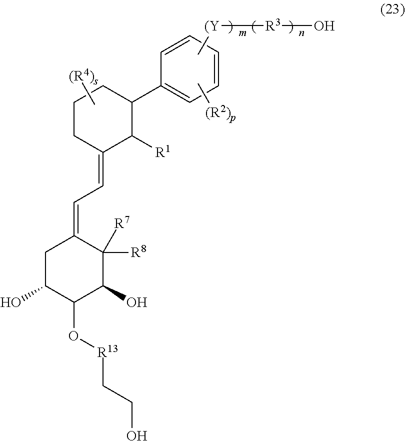

In Formula (23), m, n, p, s, Y, R¹, R², R³, R⁴, R⁶, R⁷, R⁸, and R¹³ are as defined above.

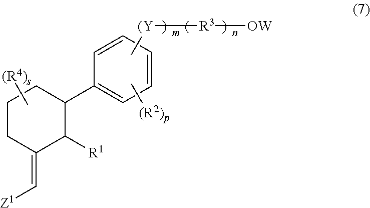

In Formula (7), m, n, p, s, W, Y, Z¹, R¹, R², R³, R⁴, and R⁶ are as defined above.

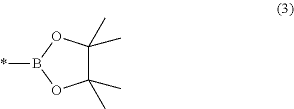 [SMILES: *B1OC(C)(C)C(C)(C)O1]

In Formula (3), * indicates a bond.

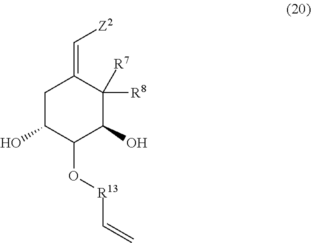

In Formula (20), Z², R⁷, R⁸, and R¹³ are as defined above.

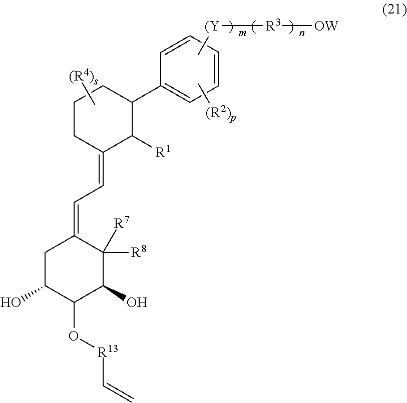

In Formula (21), m, n, p, s, W, Y, R¹, R², R³, R⁴, R⁶, R⁷, R⁸, and R¹³ are as defined above.

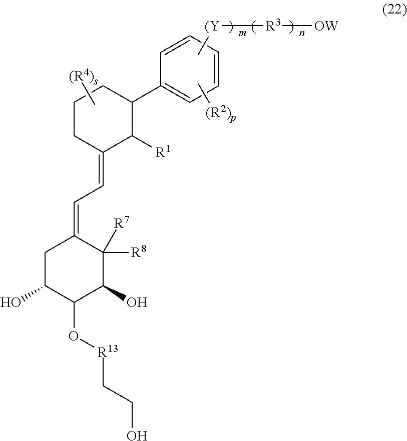

In Formula (22), m, n, p, s, Y, R¹, R², R³, R⁴, R⁶, R⁷, R⁸, R¹³, and W are as defined above.

A method for producing a compound of Formula (27) below includes allowing a compound of Formula (6) below to react with a compound of Formula (24) below to form a compound of Formula (25) below; removing the groups represented by W from the compound of Formula (25) to form a compound of Formula (26) below; and obtaining the compound of Formula (27) from the compound of Formula (26).

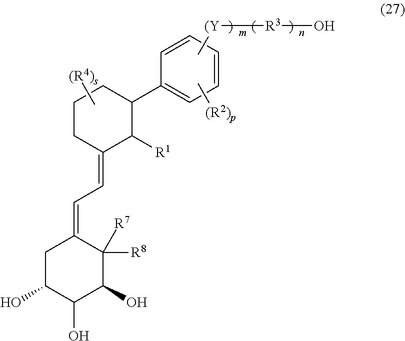

In Formula (27), m and n are independently 0 or 1, p is an integer of 0 to 4, and s is an integer of 0 to 4. Y is an oxygen atom, a sulfur atom, or a divalent group represented by —NR⁶—. R¹ is a linear or branched, saturated or unsaturated, monovalent aliphatic hydrocarbon group having 1 to 6 carbon atoms. R² is an alkyl group having 1 to 8 carbon atoms, a hydroxyl group, an alkoxy group having 1 to 8 carbon atoms, or a halogen atom. R³ is a linear or branched, saturated or unsaturated, divalent aliphatic hydrocarbon group having 1 to 20 carbon atoms, optionally having a substituent, and optionally having a heteroatom-containing carbon chain. R⁴ is a linear or branched, saturated or unsaturated, monovalent aliphatic hydrocarbon group having 1 to 6 carbon atoms. R⁶ is a hydrogen atom or a monovalent aliphatic hydrocarbon group. R⁷ and R⁸ are simultaneously hydrogen atoms or combined to form a methylidene group.

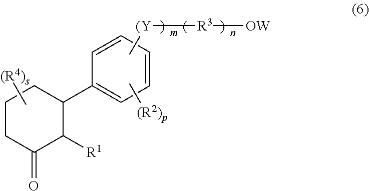

In Formula (6), m, n, p, s, Y, R¹, R², R³, R⁴, and R⁶ are as defined above. W is a protecting group.

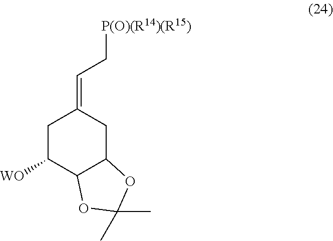

In Formula (24), W is as defined above. R¹⁴ and R¹⁵ are independently a monovalent hydrocarbon group having 1 to 20 carbon atoms.

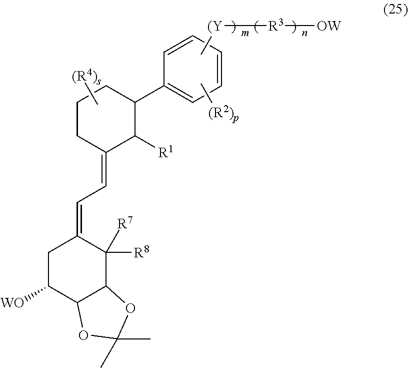

In Formula (25), m, n, p, s, Y, R¹, R², R³, R⁴, R⁶, R⁷, R⁸, and W are as defined above.

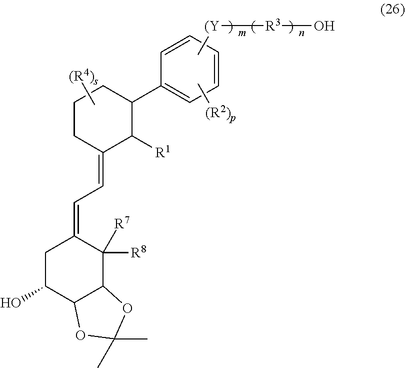

In Formula (26), m, n, p, s, Y, R¹, R², R³, R⁴, R⁶, R⁷, and R⁸ are as defined above.

### EXAMPLES

Hereinafter, the present invention will be specifically described with reference to examples, which are not intended to limit the present invention.

### Synthesis of Compounds

### Synthesis Example 1

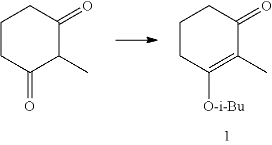

According to the method described in the document: Levine, et al., Organic Letters, 2009, 11, 289-292, 3-isobutoxy-2-methylcyclohex-2-en-1-one was prepared from 2-methylcyclohexane-1,3-dione, which was commercially available.

### Synthesis Example 2

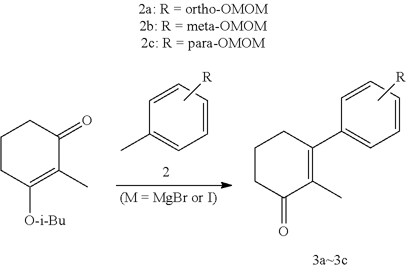

### Synthesis Example 2-1

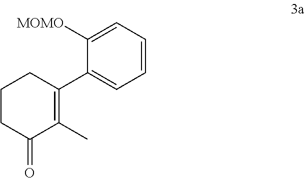

Under an argon atmosphere at −78° C., a 1.55 M hexane solution of n-butyl lithium (43.4 mL, 67.2 mmol, 1.4 equivalents) was added dropwise to a solution of 1-iodo-2-(methoxymethoxy)benzene (16.9 g, 64.0 mmol, 1.3 equivalents) in tetrahydrofuran (128 mL). The mixture was stirred at 0° C. for 30 minutes and then cooled to −78° C. To the mixture was added 3-isobutoxy-2-methylcyclohex-2-en-1-none (9.0 g, 49.2 mmol, 1.0 equivalent) and stirred for 1.5 hours. The reaction mixture was quenched by adding 1 M hydrochloric acid, followed by extraction with ethyl acetate. The organic layer was washed with a saturated aqueous sodium bicarbonate solution and then a saturated aqueous sodium chloride solution, then dried over anhydrous magnesium sulfate, and then filtered through Celite. The filtrate was concentrated under reduced pressure. The resulting residue was purified by silica gel column chromatography to give compound 3a (9.3 g) in a yield of 58%.

¹H NMR (600 MHz, CDCl₃) δ 7.25-7.28 (m, 1H, Ar), 7.15 (d, J=8.4 Hz, 1H, Ar), 7.01-7.03 (m, 2H, Ar), 5.15 (s, 2H, OCH₂O), 3.44 (s, 3H, CH₃O), 2.42-2.68 (m, 4H, CH₂), 2.04-2.12 (m, 2H, CH₂CH₂CH₂), 1.61 (t, J=1.8 Hz, 3H, CH₃C═C). ¹³C NMR (150 MHz, CDCl₃) δ 200.0, 155.1, 152.9, 132.8, 131.1, 129.0, 128.3, 121.8, 114.8, 94.5, 56.1, 38.0, 32.3, 22.9, 12.7.

IR (ATR) 2948, 2825, 1664, 1625, 1487, 1450, 1440, 1353, 1239, 1195, 1153, 1117, 1102, 1079, 1041, 995, 973, 923, 756 cm⁻¹.
HR-MS m/z=calcd for C₁₅H₁₈NaO₃ [M+Na]⁺: 269.11536, found 269.11431.

### Synthesis Example 2-2

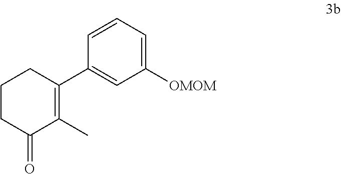

Under an argon atmosphere, 1-bromo-3-(methoxymethoxy)benzene (12.7 g, 58.7 mmol, 1.3 equivalents) was added dropwise to a mixture of magnesium shavings (2.1 g, 88 mmol, 3.0 equivalents) and tetrahydrofuran (58.7 mL). The mixture was stirred for 30 minutes. At 0° C., 3-isobutoxy-2-methylcyclohex-2-en-1-none (8.2 g, 45.1 mmol, 1.0 equivalent) was added to the resulting Grignard reagent and stirred for 1.5 hours. The reaction mixture was quenched by adding 1 M hydrochloric acid, followed by extraction with ethyl acetate. The resulting organic layer was washed with a saturated aqueous sodium bicarbonate solution and then a saturated aqueous sodium chloride solution, then dried over anhydrous magnesium sulfate, and then filtered through Celite. The filtrate was concentrated under reduced pressure. The resulting residue was purified by silica gel column chromatography to give compound 3b (8.5 g) in a yield of 76%.

¹H NMR (600 MHz, CDCl₃) δ 7.31 (t, J=7.8 Hz, 1H, Ar), 7.00 (dd, J=3.0, 7.8 Hz, 1H, Ar), 6.87 (dd, 1H, J=1.2, 3.0 Hz, Ar), 6.83 (dd, J=1.2, 7.8 Hz, 1H, Ar), 5.19 (s, 2H, OCH₂O), 3.50 (s, 3H, CH₃O), 2.63-2.61 (m, 2H, CH₂), 2.52 (t, J=6.0 Hz, 2H, CH₂), 2.09 (quint, J=6.5 Hz, 2H, CH₂), 1.73 (t, J=1.8 Hz, 3H, CH₃C═C).

¹³C NMR (150 MHz, CDCl₃) δ 200.0, 157.2, 156.2, 142.8, 131.9, 129.5, 120.6, 115.5, 115.1, 94.5, 56.1, 37.8, 32.8, 22.8, 12.9.
IR (ATR) 2948, 2825, 1662, 1598, 1576, 1483, 1432, 1352, 1306, 1262, 1149, 1107, 1078, 1017, 979, 921, 875, 787, 701 cm⁻¹.
HR-MS m/z=calcd for C₁₅H₁₈NaO₃ [M+Na]⁺: 269.11536, found 269.11526.

### Synthesis Example 2-3

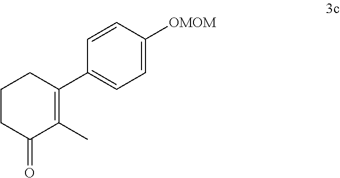

Similar to the synthesis of compound 3b, compound 3c (10.9 g) was synthesized in a yield of 76% from compound 1 (10.6 g, 58.1 mmol).

¹H NMR (600 MHz, CDCl₃) δ 7.15 (d, J=9.0 Hz, 2H, Ar), 7.06 (d, J=9.0 Hz, 2H, Ar), 5.20 (s, 2H, OCH₂O), 3.50 (s, 3H, CH₃O), 2.63-2.59 (m, 2H, CH₂), 2.51 (t, J=6 Hz, 2H, CH₂), 2.08 (quint, J=6.3 Hz, 2H, CH₂), 1.75 (t, J=1.8 Hz, 3H, CH₃C═C).

¹³C NMR (150 MHz, CDCl₃) δ 200.1, 156.9, 156.1, 134.7, 131.7, 128.7, 115.9, 94.4, 56.1, 37.8, 32.9, 22.7, 13.0.
IR (ATR) 2948, 2826, 1660, 1606, 1508, 1439, 1353, 1234, 1197, 1176, 1151, 1104, 1078, 996, 974, 922, 835 cm⁻¹.
HR-MS m/z=calcd for C₁₅H₁₈NaO₃ [M+Na]⁺: 269.11536, found 269.11711.

### Synthesis Example 3

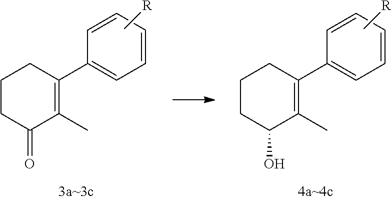

### Synthesis Example 3-1

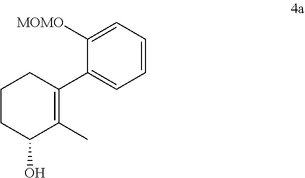

Under an argon atmosphere, (S)-α,α-diphenylprolinol (0.76 g, 3.0 mmol, 10.0 mol %) and trimethyl borate (0.4 mL, 3.6 mmol, 12.0 mol %) were dissolved in tetrahydrofuran (50 mL). After the resulting tetrahydrofuran solution was stirred at room temperature for 1 hour, N,N-diethylaniline borane (5.6 mL, 31.5 mmol, 1.05 equivalents) was added to the solution. After the resulting mixture was cooled to −10° C., a solution of compound 3a (7.4 g, 30.0 mmol, 1.0 equivalent) in tetrahydrofuran (50 mL) was added dropwise to the mixture over 45 minutes. The resulting mixture was stirred at −5° C. for 12 hours. The reaction mixture was quenched by adding 1 M hydrochloric acid, followed by extraction with diethyl ether. The resulting organic layer was washed with a saturated aqueous sodium chloride solution, then dried over anhydrous sodium sulfate, and then filtered, and the filtrate was concentrated. The resulting residue was purified by silica gel column chromatography (hexane/ethyl acetate) to give compound (R)-4a (6.8 g) in a yield of 91%.

¹H NMR (600 MHz, CDCl₃) δ 7.19 (t, J=8.4 Hz, 1H, Ar), 7.09 (d, J=8.4 Hz, 1H, Ar), 7.09-6.96 (m, 2H, Ar), 5.12 (s, 1H, OCH₂O), 4.18-4.06 (m, 1H, OCH), 3.45 (s, 3H, CH₃O), 2.39-1.62 (m, 6H, OH and CH₂), 1.59 (t, J=1.8 Hz, 3H, CH₃C═C), 1.55-1.47 (m, 1H, CH₂).

¹³C NMR (150 MHz, CDCl₃) δ 154.2 and 153.9 (rotamers), 134.0, 133.2, 131.2, 130.2 and 127.8 (rotamers), 127.6, 121.9, 115.5 and 115.4 (rotamers), 94.9, 69.5 and 69.3 (rotamers), 55.9, 32.3 and 32.1 (rotamers), 31.4, 18.9 and 18.4 (rotamers), 17.5.
IR (ATR) 3393, 2933, 2860, 1597, 1578, 1487, 1440, 1240, 1193, 1151, 1114, 1076, 1046, 1002, 957, 922, 753 cm⁻¹.
HR-MS m/z=calcd for C₁₅H₂₀NaO₃ [M+Na]⁺: 271.13101, found 271.13283.

### Synthesis Example 3-2

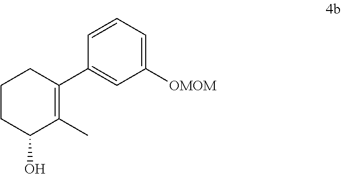

Similar to the synthesis of compound (R)-4a from compound 3a, compound (R)-4b (4.6 g) was synthesized in a yield of 93% from compound 3b (4.9 g, 19.9 mmol).

¹H NMR (600 MHz, CDCl₃) δ 7.24 (t, J=7.8 Hz, 1H, Ar), 6.91 (dd, J=2.4, 8.4 Hz, 1H, Ar), 6.82 (t, J=1.8 Hz, 1H, Ar), 6.78 (d, J=7.8 Hz, 1H, Ar), 5.17 (s, 2H, OCH₂O), 4.12 (brs, 1H, CHO), 3.49 (s, 3H, OCH₃), 2.31-2.17 (m, 2H, CH₂), 1.87-1.77 (m, 3H, CH₂), 1.72-1.67 (m, 1H, CH₂), 1.70 (t, J=1.8 Hz, 3H, CH₃C═C), 1.53 (brs, 1H, OH).

¹³C NMR (150 MHz, CDCl₃) δ 157.1, 144.8, 136.5, 130.5, 129.1, 121.8, 116.1, 114.2, 94.5, 69.4, 56.0, 32.2, 32.0, 18.6, 17.7.
IR (ATR) 3362, 2931, 2861, 2827, 1598, 1577, 1483, 1434, 1275, 1150, 1077, 1013, 983, 963, 922, 787, 703 cm⁻¹.
HR-MS m/z=calcd for C₁₅H₂₀NaO₃ [M+Na]⁺: 271.13101, found 271.13326.

### Synthesis Example 3-3

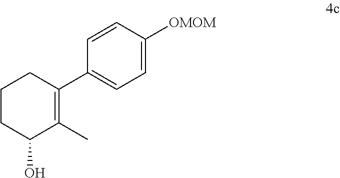

Similar to the synthesis of compound (R)-4a from compound 3a, compound (R)-4c (10.7 g) was synthesized in a yield of 93% from compound 3c (10.9 g, 44.3 mmol).

¹H NMR (600 MHz, CDCl₃) δ 7.06 (d, J=9.0 Hz, 2H, Ar), 6.99 (d, J=9.0 Hz, 2H, Ar), 5.18 (s, 2H, OCH₂O), 4.12 (brs, 1H, CHO), 3.50 (s, 3H, OCH₃), 2.27-2.18 (m, 2H, CH₂), 1.87-1.77 (m, 3H, CH₂), 1.74-1.67 (m, 1H, CH₂), 1.70 (t, J=1.8 Hz, 3H, CH₃), 1.53 (brs, 1H, OH).

¹³C NMR (150 MHz, CDCl₃) δ 155.7, 136.9, 136.2, 130.3, 129.2, 115.8, 94.5, 69.5, 56.0, 32.3, 32.0, 18.7, 17.7.
IR (ATR) 3389, 2932, 2861, 2827, 1606, 1508, 1442, 1230, 1198, 1151, 1077, 1004, 958, 922, 834 cm⁻¹.
HR-MS m/z=calcd for C₁₅H₂₀NaO₃ [M+Na]⁺: 271.13101, found 271.13371.

### Synthesis Example 4

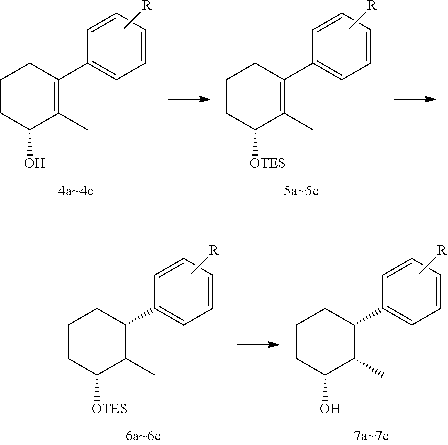

### Synthesis Example 4-1

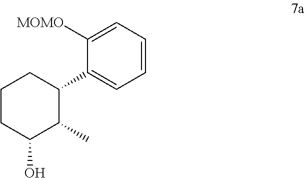

Chlorotriethylsilane (3.4 mL, 20.4 mmol, 1.5 equivalents) was added at 0° C. to a solution of compound 4a (3.4 g, 13.6 mmol, 1.0 equivalent) and imidazole (2.0 g, 29.9 mmol, 2.2 equivalents) in N,N-dimethylformamide (27.2 mL). After the mixture was stirred at room temperature for 3 hours, a saturated aqueous sodium bicarbonate solution was added at 0° C. to the reaction mixture, followed by extraction with hexane. The organic layer was dried over anhydrous magnesium sulfate and then filtered. The filtrate was concentrated to give a crude product of compound 5a.

Palladium (supported on charcoal, 0.25 g) was added in an amount of 10 mass % to a solution of the crude product of compound 5a in methanol (68 mL). The mixture was stirred at room temperature (or 40° C.) under a hydrogen atmosphere for 12 hours. After the resulting mixture was filtered through Celite, the filtrate was concentrated to give a crude product of compound 6a.

A 1 M tetrahydrofuran solution of tetrabutylammonium fluoride (20.4 mL, 20.4 mmol, 1.5 equivalents) was added at 0° C. to a solution of the crude product of compound 6a in tetrahydrofuran (13.6 mL). After the mixture was stirred at room temperature for 3 hours, a saturated aqueous ammonium chloride solution was added to the reaction mixture, followed by extraction with diethyl ether. The resulting organic layer was dried over anhydrous sodium sulfate and then filtered, and the filtrate was concentrated. The resulting residue was purified by silica gel column chromatography (hexane/ethyl acetate) to give compound 7a (d.r. 92:8) (2.7 g) in a yield of 80%.

¹H NMR (600 MHz, CDCl₃) δ 7.14 (t, J=7.8 Hz, 1H, Ar), 7.11 (d, J=7.2 Hz, 1H, Ar), 7.07 (d, J=8.4 Hz, 1H, Ar), 6.96 (t, J=7.8 Hz, 1H, Ar), 5.20 (d, J=6.0 Hz, 1H, OCH₂O), 5.18 (d, J=6.0 Hz, 1H, OCH₂O), 3.96 (dt, J=11.4, 4.2 Hz, 1H, CHO), 3.49 (s, 3H, OCH₃), 3.22 (dt, J=13.2, 3.0 Hz, 1H, CHAr), 2.41-2.34 (m, 1H, CHCH₃), 1.90-1.70 (m, 3H, CH₂), 1.55-1.41 (m, 4H, CH₂), 1.34 (brs, 1H, OH), 0.66 (d, J=7.8 Hz, 3H, CHCH₃).

¹³C NMR (150 MHz, CDCl₃) δ 154.5, 133.2, 128.1, 126.8, 121.2, 113.7, 94.5, 73.7, 56.1, 39.1, 38.2, 28.7, 24.4, 23.2, 5.9.
IR (ATR) 3399, 2931, 2863, 2825, 1723, 1600, 1584, 1489, 1449, 1291, 1229, 1202, 1184, 1152, 1074, 1006, 924, 753 cm⁻¹.
HR-MS m/z=calcd for C₁₅H₂₂NaO₃ [M+Na]⁺: 273.14666, found 273.14538.

### Synthesis Example 4-2

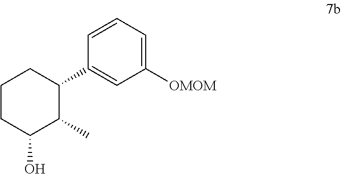

Similar to the synthesis of compound 7a from compound (R)-4a, compound 7b (2.3 g) was synthesized in a yield of 91% from compound (R)-4b (2.6 g, 10.1 mmol).

¹H NMR (600 MHz, CDCl₃) δ 7.21 (t, J=7.8 Hz, 1H, Ar), 6.88 (d, J=8.4 Hz, 1H, Ar), 6.85 (s, 1H, Ar), 6.83 (d, 1H, J=7.8 Hz, Ar), 5.17 (d, J=6.6 Hz, H, 1), 5.16 (d, J=6.6 Hz, 1H, OCH₂O), 3.92 (dt, J=12.0, 4.2 Hz, 1H, CHO), 3.48 (s, 3H, OCH₃), 2.79 (dt, J=13.2, 3.6 Hz, 1H, CHAr), 2.28-2.22 (m, 1H, CHCH₃), 1.92-1.86 (m 1H, CH₂), 1.78-1.68 (m 2H, CH₂), 1.59 (dd, J=12.6, 2.4 Hz, 1H, CH₂), 1.55 (brs, 1H, OH), 1.49 (ddd, J=12.0, 4.2, 3.6 Hz, 1H, CH₂), 1.40 (tq, 4.2, 13.2 Hz, 1H, CH₂), 0.66 (d, J=7.2 Hz, 3H, CHCH₃).

¹³C NMR (150 MHz, CDCl₃) δ 157.2, 146.4, 129.0, 121.1, 115.7, 113.4, 94.5, 73.7, 55.9, 45.5, 41.4, 28.4, 24.1, 22.9, 5.5.
IR (ATR) 3383, 2931, 2863, 2825, 1601, 1583, 1487, 1445, 1240, 1150, 1076, 1011, 995, 944, 922, 793, 775, 699 cm⁻¹.
HR-MS m/z=calcd for C₁₅H₂₂NaO₃ [M+Na]⁺: 273.14666, found 273.14538.

### Synthesis Example 4-3

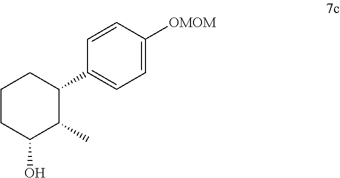

Similar to the synthesis of compound 7a from compound (R)-4a, compound 7c (4.5 g) was synthesized in a yield of 97% from compound (R)-4c (5.8 g, 23.5 mmol).

¹H NMR (600 MHz, CDCl₃) δ 7.09 (d, J=7.8 Hz, 2H, Ar), 6.97 (d, J=7.8 Hz, 2H, Ar), 5.16 (s, 2H, OCH₂O), 3.93-3.91 (m, 1H, CHO), 3.48 (s, 3H, OCH₃), 2.77 (dt, J=13.2, 3.6 Hz, 1H, CHAr), 2.24-2.18 (m, 1H, CHCH₃), 1.91-1.85 (m, 1H, CH₂), 1.74 (dd, J=4.2, 13.2 Hz, 1H, CH₂), 1.70 (dd, J=3.6, 12.6 Hz, 1H, CH₂), 1.58 (brs, 1H, OH), 1.49 (dq, J=4.2, 12.0 Hz, 1H, CH₂), 1.45-1.36 (m, 2H, CH₂), 0.65 (d, J=7.8 Hz, 3H, CHCH₃).

¹³C NMR (150 MHz, CDCl₃) δ 155.4, 138.0, 128.4, 115.9, 94.6, 73.8, 56.0, 44.9, 41.6, 28.5, 24.2, 23.2, 5.4.
IR (ATR) 3395, 2931, 2895, 2862, 2826, 1610, 1509, 1467, 1446, 1232, 1199, 1177, 1151, 1077, 1008, 924, 831 cm⁻¹.
HR-MS m/z=calcd for C₁₅H₂₂NaO₃ [M+Na]⁺: 273.14666, found 273.14542.

### Synthesis Example 5

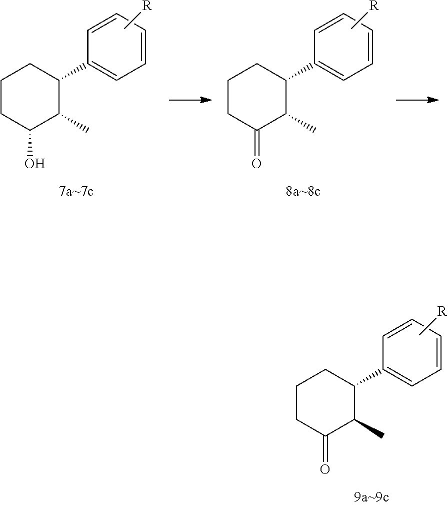

### Synthesis Example 5-1

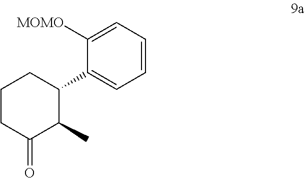

Pyridinium chlorochromate (1.1 g, 5.1 mmol, 1.5 equivalents) and Celite (1.1 g) were added at 0° C. to a solution of compound 7a (0.85 g, 3.4 mmol, 1.0 equivalent) in methylene chloride (34 mL) and stirred at room temperature for 3 hours. After the solid was filtered off, the filtrate was concentrated to give a crude product of compound 8a. Potassium carbonate (1.4 g, 10.2 mmol, 3.0 equivalents) was added to a solution of the crude product of compound 8a in methanol (15 mL). After the mixture was stirred at room temperature for 24 hours, water was added to the mixture. After the mixture was extracted with hexane, the organic layer was dried over anhydrous magnesium sulfate. After the organic layer was filtered, the filtrate was concentrated to give compound 9a (0.77 g) in a yield of 91%.

¹H NMR (600 MHz, CDCl₃) δ 7.22 (d, J=7.8 Hz, 1H, Ar), 7.18 (t, J=7.8 Hz, 1H, Ar), 7.09 (d, J=7.8 Hz, 1H, Ar), 7.01 (t, J=7.8 Hz, 1H, Ar), 5.20 (d, J=7.2 Hz, 1H, OCH₂O), 5.17 (d, J=7.2 Hz, 1H, OCH₂O), 3.46 (s, 3H, OCH₃), 3.15 (brt, J=10.2 Hz, 1H, CH₂), 2.79-2.72 (m, 1H, CH₂), 2.53-2.48 (m, 2H, CH₂ and CHCH₃), 2.46 (dd, J=6.0, 13.2 Hz, 1H, CHAr), 2.17-2.11 (m, 1H, CH₂), 2.03-1.92 (m, 2H, CH₂), 1.82-1.73 (m, 1H, CH₂), 0.83 (d, J=6.6 Hz, 3H, CHCH₃).

¹³C NMR (150 MHz, CDCl₃) δ 212.9, 154.7, 132.5, 127.5, 127.4, 122.0, 114.2, 94.4, 56.0, 49.8, 45.3, 41.9, 33.0, 26.6, 12.0.
IR (ATR) 2933, 2865, 2826, 1708, 1600, 1585, 1491, 1453, 1233, 1201, 1182, 1153, 1078, 1045, 1001, 924, 755 cm⁻¹.
HR-MS m/z=calcd for C₁₅H₂₀NaO₃ [M+Na]⁺. 271.13101, found 271.13008.

### Synthesis Example 5-2

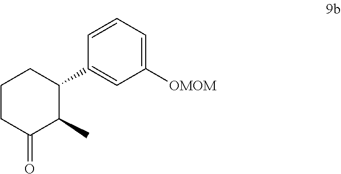

Similar to the synthesis of compound 9a from compound 7a, compound 9b (3.2 g) was synthesized in a yield of 93% from compound 7b (3.5 g, 13.9 mmol).

¹H NMR (600 MHz, CDCl₃) δ 7.24 (t, J=8.1 Hz, 1H, Ar), 6.92 (dd, J=2.4, 8.4 Hz, 1H, Ar), 6.87 (s, 1H, Ar), 6.85 (d, J=7.2 Hz, 1H, Ar), 5.18 (d, J=7.2 Hz, 1H, OCH₂O), 5.17 (d, J=7.2 Hz, 1H, OCH₂O), 3.49 (s, 3H, OCH₃), 2.61 (dq, J=12.0, 6.0 Hz, 1H, CHCH₃), 2.52 (dd, J=4.2, 12.0 Hz, 1H, CH₂), 2.51-2.48 (m, 1H, CHAr), 2.46 (dd, J=6.0, 13.8 Hz, 1H, CH₂), 2.17-2.12 (m, 1H, CH₂), 2.00 (dd, J=3.0, 13.8 Hz, 1H, CH₂), 1.93 (dq, J=3.6, 12.0 Hz, 1H, CH₂), 1.75 (tq, J=4.2, 12.6 Hz, 1H, CH₂), 0.83 (d, J=6.0 Hz, 3H, CHCH₃).

¹³C NMR (150 MHz, CDCl₃) δ 212.3, 157.6, 145.6, 129.6, 120.8, 115.3, 114.1, 94.5, 56.0, 53.1, 50.5, 41.8, 34.4, 26.5, 12.3.
IR (ATR) 2933, 2871, 2826, 1708, 1599, 1584, 1487, 1449, 1260, 1246, 1150, 1079, 1015, 995, 950, 923, 785, 701 cm⁻¹.
HR-MS m/z=calcd for C₁₅H₂₀NaO₃ [M+Na]⁺: 271.13101, found 271.13039.

### Synthesis Example 5-3

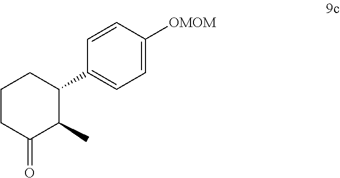

Similar to the synthesis of compound 9a from compound 7a, compound 9c (1.13 g) was synthesized in a yield of 91% from compound 7c (1.25 g, 5.0 mmol).

¹H NMR (600 MHz, CDCl₃) δ 7.11 (d, J=8.4 Hz, 2H, Ar), 7.00 (d, J=8.4 Hz, 2H, Ar), 5.17 (s, 2H, OCH₂O), 3.49 (s, 3H, OCH₃), 2.58 (dq, J=12.0, 6.6 Hz, 1H, CHCH₃), 2.50 (dd, J=3.0, 12.0 Hz, 1H, CH₂), 2.49-2.48 (m, 1H, CHAr), 2.44 (dd, J=6.0, 13.2 Hz, 1H, CH₂), 2.17-2.11 (m, 1H, CH₂), 1.97 (dd, J=3.0, 13.2 Hz, 1H, CH₂), 1.91 (dq, J=3.6, 13.2 Hz, 1H, CH₂), 1.75 (tq, J=4.8, 12.6 Hz, 1H, CH₂), 0.81 (d, J=6.0 Hz, 3H, CHCH₃).

¹³C NMR (150 MHz, CDCl₃) δ 212.4, 155.9, 137.3, 128.1, 116.3, 94.5, 56.0, 52.4, 50.8, 41.8, 34.6, 26.4, 12.2.
IR (ATR) 2933, 2825, 1708, 1610, 1510, 1446, 1309, 1234, 1199, 1178, 1152, 1078, 1003, 923, 832 cm⁻¹.
HR-MS m/z=calcd for C₁₅H₂₀NaO₃ [M+Na]⁺: 271.13101, found 271.13152.

### Synthesis Example 6

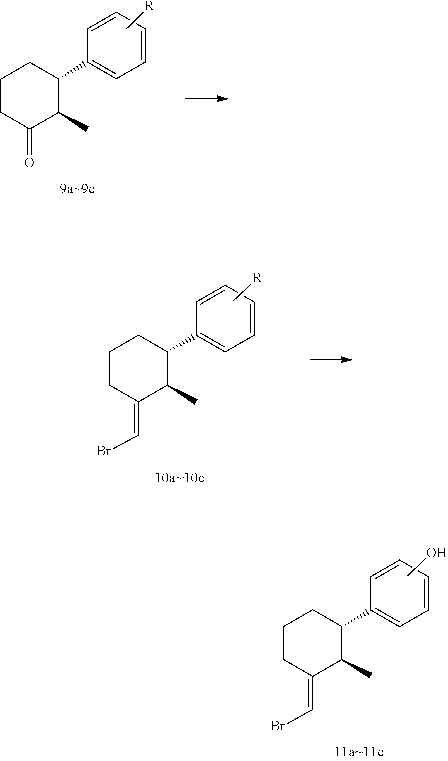

### Synthesis Example 6-1

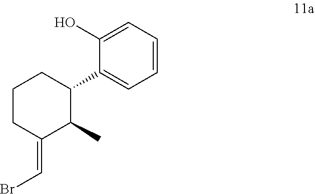 [SMILES: C[C@H]1/C(=C/Br)CCC[C@@H]1c1ccccc1O]

A 0.5 M tetrahydrofuran solution of lithium diisopropylamide (23.0 mL, 11.5 mmol, 1.4 equivalents) was added at −78° C. to a mixture of (bromomethyl)triphenylphosphonium bromide (5.4 g, 12.3 mmol, 1.5 equivalents) and tetrahydrofuran (12.0 mL). The mixture was heated to 0° C. and stirred for 1 hour. After the mixture was cooled again to −78° C., a solution of compound 9a (2.0 g, 8.05 mmol, 1.0 equivalent) in tetrahydrofuran (41 mL) was added to the mixture. The resulting mixture was stirred for 12 hours while being heated at 0° C. The reaction mixture was filtered through silica gel with hexane. The resulting filtrate was concentrated under reduced pressure to give a crude product of compound 10a.

At 0° C., 3 M hydrochloric acid (8.0 mL) was added to a solution of the crude product of compound 10a in methanol (16.0 mL). After the mixture was stirred for 24 hours, the reaction was quenched by adding a saturated aqueous sodium bicarbonate solution, followed by extraction with diethyl ether. The resulting organic layer was washed with a saturated aqueous sodium chloride solution, then dried over anhydrous sodium sulfate, and then filtered, and the filtrate was concentrated. The resulting residue was purified by silica gel column chromatography (hexane/ethyl acetate) to give compound 11a (2.0 g) in a yield of 88%.

¹H NMR (600 MHz, CDCl₃) δ 7.17 (d, J=7.8 Hz, 1H, Ar), 7.07 (t, J=7.8 Hz, 1H, Ar), 6.91 (t, J=7.8 Hz, 1H, Ar), 6.72 (d, J=7.8 Hz, 1H, Ar), 5.94 (s, 1H, C═CHBr), 4.67 (brs, 1H, OH), 3.10 (brd, 1H, CHAr), 2.68 (dt, J=3.6, 12.0 Hz, 1H, CHCH₃), 2.51-2.44 (m, 1H, allylic CH₂), 1.95-1.84 (m, 3H, CH₂), 1.76 (dq, J=3.0, 12.6 Hz, 1H, CH₂), 1.46 (tq, J=3.6, 12.6 Hz, 1H, CH₂), 0.87 (d, J=6.6 Hz, 3H, CHCH₃).

¹³C NMR (150 MHz, CDCl₃) δ 153.0, 148.5, 131.1, 127.8, 126.9, 121.1, 115.5, 98.9, 45.4, 43.1, 33.6, 32.4, 26.6, 15.3.
IR (ATR) 3526, 2963, 2929, 2855, 1590, 1501, 1489, 1451, 1376, 1330, 1269, 1251, 1235, 1209, 1172, 1127, 1082, 1039, 870, 848, 826, 802, 782, 751, 707 cm⁻¹.
HR-MS m/z=calcd for C₁₄H₁₆BrO [M−H]⁻: 279.03845, found 279.03320.

### Synthesis Example 6-2

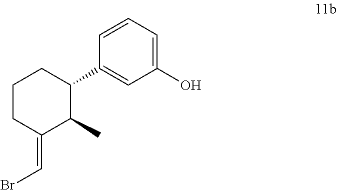 [SMILES: C[C@H]1/C(=C/Br)CCC[C@@H]1c1cccc(O)c1]

Similar to the synthesis of compound 11a from compound 9a, compound 11b (2.9 g) was synthesized in a yield of 90% from compound 9b (2.5 g, 10.0 mmol).

¹H NMR (600 MHz, CDCl₃) δ 7.16 (t, J=7.8 Hz, 1H, Ar), 6.74 (d, J=7.8 Hz, 1H, Ar), 6.67 (d, J=9.0 Hz, 1H, Ar), 6.65 (s, 1H, Ar), 5.93 (s, 1H, C═CHBr), 4.59 (s, 1H, OH), 3.10 (brd, J=11.4 Hz, 1H, CHAr), 2.32 (dq, J=11.4, 6.6 Hz, 1H, CHCH₃), 2.18 (dt, J=3.6, 12.0 Hz, 1H, CH₂), 1.94-1.86 (m, 3H, CH₂), 1.67 (dq, J=3.0, 12.6 Hz, 1H, CH₂), 1.43 (tq, J=3.6, 12.6 Hz, 1H, CH₂), 0.83 (d, J=6.6 Hz, 3H, CHCH₃).

¹³C NMR (150 MHz, CDCl₃) δ 155.6, 148.3, 147.3, 129.6, 120.0, 114.2, 113.2, 99.1, 53.2, 43.7, 35.2, 32.4, 26.6, 15.7.
IR (ATR) 3338, 2962, 2928, 2875, 2854, 1613, 1590, 1491, 1456, 1376, 1330, 1277, 1247, 1156, 970, 885, 856, 834, 801, 784, 761, 699 cm⁻¹.
HR-MS m/z=calcd for C₁₄H₁₆BrO [M−H]⁻: 279.03845, found 279.03489.

### Synthesis Example 6-3

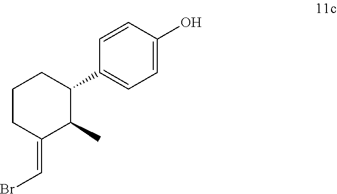 [SMILES: C[C@H]1/C(=C/Br)CCC[C@@H]1c1ccc(O)cc1]

Similar to the synthesis of compound 11a from compound 9a, compound 11c (0.3 g) was synthesized in a yield of 87% from compound 9c (0.3 g, 1.2 mmol).

¹H NMR (600 MHz, CDCl₃) δ 7.03 (d, J=8.4 Hz, 2H, Ar), 6.77 (d, J=8.4 Hz, 2H, Ar), 5.92 (s, 1H, C═CHBr), 4.65 (s, 1H, OH), 3.09 (brd, J=15.0 Hz, 1H, CHAr), 2.28 (dq, J=12.0, 6.6 Hz, 1H, CHCH₃), 2.17 (dt, J=3.6, 12.0 Hz, 1H, CH₂), 1.94-1.83 (m, 3H, CH₂), 1.65 (dq, J=3.6, 12.6 Hz, 1H, CH₂), 1.42 (tq, J=3.6, 12.6 Hz, 1H, CH₂), 0.81 (d, J=6.6 Hz, 3H, CHCH₃).

¹³C NMR (150 MHz, CDCl₃) δ 153.8, 148.5, 137.7, 128.4, 115.2, 98.9, 52.5, 44.0, 35.5, 32.4, 26.6, 15.7.
IR (ATR) 3334, 2962, 2927, 2873, 2854, 1612, 1598, 1512, 1455, 1443, 1375, 1331, 1227, 1173, 849, 827, 800, 783, 763, 708 cm⁻¹.
HR-MS m/z=calcd for C₁₄H₁₆BrO [M−H]⁻: 279.03845, found 279.03612.

### Synthesis Example 7

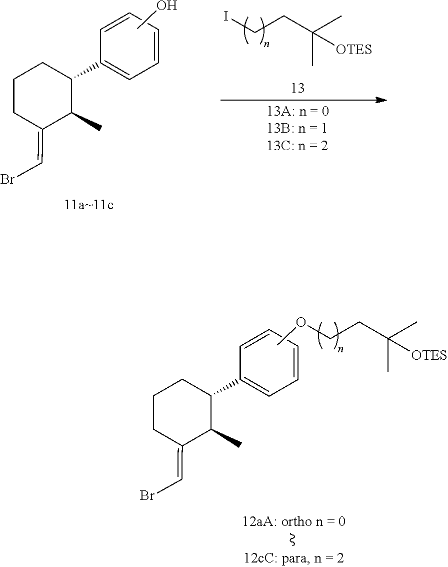

### Synthesis Example 7-1

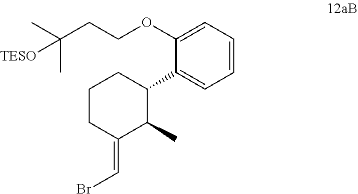

Anhydrous potassium carbonate (0.47 g, 3.4 mmol, 4.0 equivalents) and triethyl((4-iodo-2-methylbutan-2-yl)oxy)silane (13B) (1.1 g, 3.4 mmol, 4.0 equivalents) were added to a solution of compound 11a (0.24 g, 0.85 mmol, 1.0 equivalent) in N,N-dimethylformamide (1.7 mL). The mixture was stirred at 50° C. for 24 hours. After the reaction mixture was cooled to room temperature, water was added to the reaction mixture, followed by extraction with diethyl ether. The resulting organic layer was washed with a saturated aqueous sodium chloride solution, then dried over anhydrous magnesium sulfate, and then filtered, and the filtrate was concentrated. The resulting residue was purified by silica gel column chromatography (hexane/ethyl acetate) to give compound 12aB (0.33 g) in a yield of 81%.

¹H NMR (600 MHz, CDCl₃) δ 7.16 (d, J=7.8 Hz, 1H, Ar), 7.15 (d, J=7.2 Hz, 1H, Ar), 6.91 (t, J=7.2 Hz, 1H, Ar), 6.87 (d, J=7.2 Hz, 1H, Ar), 5.92 (s, 1H, C═CHBr), 4.10 (t, J=7.2 Hz, 2H, OCH₂), 3.09 (brd, J=13.8 Hz, 1H, CHAr), 2.78 (brt, J=8.4 Hz, 1H, CH₂), 2.47 (brs, 1H, CH₂), 1.94 (t, J=7.2 Hz, 2H, OCH₂CH₂), 1.95-1.84 (m, 2H, CH₂ and CHCH₃), 1.80 (brd, J=12.6 Hz, 1H, CH₂), 1.72 (brq, J=11.4 Hz, 1H, CH₂), 1.43 (tq, J=4.2, 12.6 Hz, 1H, CH₂), 1.30 (s, 3H, C(CH₃)₂), 1.29 (s, 3H, C(CH₃)₂), 0.95 (t, J=7.8 Hz, 9H, Si(CH₂CH₃)₃), 0.82 (d, J=6.6 Hz, 3H, CHCH₃), 0.59 (q, J=7.8 Hz, 6H, Si(CH₂CH₃)₃).

¹³C NMR (150 MHz, CDCl₃) δ 156.6, 149.0, 133.4, 127.4, 126.8, 120.4, 111.5, 98.6, 72.4, 64.8, 43.8, 43.0, 33.8, 32.5, 30.5, 30.4, 26.7, 15.4, 7.1, 6.7.
IR (ATR) 2956, 2931, 2874, 1599, 1585, 1492, 1450, 1378, 1365, 1231, 1159, 1051, 1025, 740, 723 cm⁻¹.

### Synthesis Example 7-2

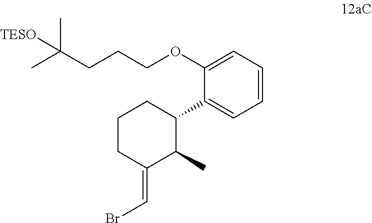

Similar to the synthesis of compound 12aB from compound 11a, compound 12aC (0.38 g) was synthesized in a yield of 89% from compound 11a (0.24 g, 0.85 mmol) and 13C.

¹H NMR (600 MHz, CDCl₃) δ 7.15 (d, J=7.8 Hz, 1H, Ar), 7.14 (d, J=7.8 Hz, 1H, Ar), 6.90 (t, J=7.8 Hz, 1H, Ar), 6.83 (d, J=7.8 Hz, 1H, Ar), 5.92 (s, 1H, C═CHBr), 3.94 (t, J=6.6 Hz, 2H, OCH₂), 3.09 (brd, J=13.8 Hz, 1H, CHAr), 2.77 (brt, J=6.9 Hz, 1H, CH₂), 2.51 (brs, 1H, CH₂), 1.93-1.72 (m, 6H, CH₂ and CHCH₃), 1.59-1.56 (m, 2H, CH₂), 1.44 (tq, J=4.2, 12.6 Hz, 1H, CH₂), 1.24 (s, 6H, C(CH₃)₂), 0.95 (t, J=7.8 Hz, 9H, Si(CH₂CH₃)₃), 0.83 (d, J=6.6 Hz, 3H, CHCH₃), 0.57 (q, J=7.8 Hz, 6H, Si(CH₂CH₃)₃).

¹³C NMR (150 MHz, CDCl₃) δ 156.7, 148.9, 133.5, 127.7, 120.5, 111.7, 98.6, 73.0, 68.7, 42.9, 41.7, 33.6, 32.5, 29.9, 26.7, 24.6, 17.7, 15.4, 7.1, 6.8.
IR (ATR) 2954, 2932, 2873, 1599, 1584, 1492, 1450, 1378, 1363, 1233, 1214, 1157, 1050, 1016, 742, 723 cm⁻¹

### Synthesis Example 7-3

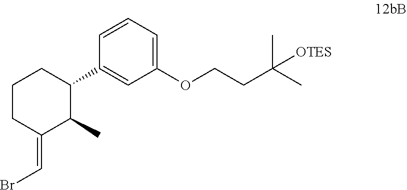

Similar to the synthesis of compound 12aB from compound 11a, compound 12bB (0.13 g) was synthesized in a yield of 88% from compound 11b (84 mg, 0.30 mmol) and 13B.

¹H NMR (600 MHz, CDCl₃) δ 7.20 (t, J=7.8 Hz, 1H, Ar), 6.75 (d, J=7.8 Hz, 1H, Ar), 6.74 (dd, J=7.8 Hz, 1H, Ar), 6.71 (s, 1H, Ar), 5.93 (s, 1H, C═CHBr), 4.12 (t, J=7.2 Hz, 2H, OCH₂CH₂), 3.10 (brd, J=13.2 Hz, 1H, CHAr), 2.37-2.30 (m, 1H, CH₂), 2.19 (dt, J=3.6, 11.4 Hz, 1H, CH₂), 1.94 (t, J=7.2 Hz, 2H, OCH₂CH₂), 1.95-1.86 (in, 4H, CH₂, and CHCH₃), 1.69 (dq, J=6.0, 12.6 Hz, 1H, CH₂), 1.43 (tq, J=3.6, 13.2 Hz, 1H, CH₂), 1.30 (s, 6H, C(CH₃)₃), 0.95 (t, J=7.8 Hz, 9H, Si(CH₂CH₃)₃), 0.83 (d, J=6.6 Hz, 3H, CHCH₃), 0.59 (q, J=7.8 Hz, 6H, Si(CH₂CH₃)₃).

¹³C NMR (150 MHz, CDCl₃) δ 159.2, 148.4, 146.9, 129.4, 119.6, 113.9, 111.8, 99.0, 72.4, 64.6, 53.4, 43.8, 43.7, 35.3, 32.5, 30.4, 30.3, 26.6, 15.7, 7.1, 6.7.
IR (ATR) 2955, 2932, 2875, 1730, 1600, 1583, 1457, 1444, 1381, 1365, 1264, 1242, 1222, 1157, 1032, 785, 743, 723, 700 cm⁻¹.

### Synthesis Example 7-4

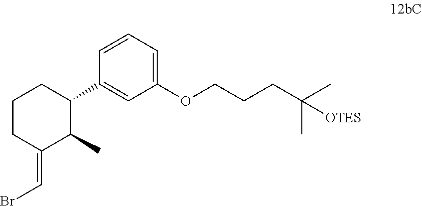

Similar to the synthesis of compound 12aB from compound 11a, compound 12bC (0.134 g) was synthesized in a yield of 90% from compound 11b (84 mg, 0.30 mmol) and 13C.

¹H NMR (600 MHz, CDCl₃) δ 7.19 (t, J=7.8 Hz, 1H, Ar), 6.76-6.69 (m, 3H, Ar), 5.93 (s, 1H, C═CHBr), 3.95 (t, J=7.2 Hz, 2H, OCH₂CH₂), 3.10 (brd, J=12.6 Hz, 1H, CHAr), 2.34 (dq, J=10.8, 6.6 Hz, 1H, CHCH₃), 2.19 (dt, J=11.4, 3.6 Hz, 1H, CH₂), 1.95-1.83 (m, 5H, CH₂ and OCH₂CH₂), 1.69 (dq, J=3.0, 12.6 Hz, 1H, CH₂), 1.59-1.56 (m, 2H, OCH₂CH₂CH₂), 1.43 (tq, J=3.0, 13.2 Hz, 1H, CH₂), 1.24 (s, 6H, C(CH₃)₂), 0.95 (t, J=8.1 Hz, 9H, Si(CH₂CH₃)₃), 0.83 (d, J=6.6 Hz, 3H, CHCH₃), 0.57 (q, J=7.8 Hz, 6H, Si(CH₂CH₃)₃).

¹³C NMR (150 MHz, CDCl₃) δ 159.3, 148.4, 146.9, 129.3, 119.6, 113.8, 111.8, 99.0, 73.0, 68.4, 53.4, 43.7, 41.3, 35.3, 32.4, 29.9, 26.6, 24.4, 15.7, 7.1, 6.8.
IR (ATR) 2954, 2932, 2874, 1729, 1600, 1583, 1456, 1444, 1380, 1363, 1262, 1238, 1215, 1156, 1052, 1034, 1015, 801, 784, 741, 721, 699, 671 cm⁻¹.

### Synthesis Example 8

In the formula below, “Me” represents a methyl group.

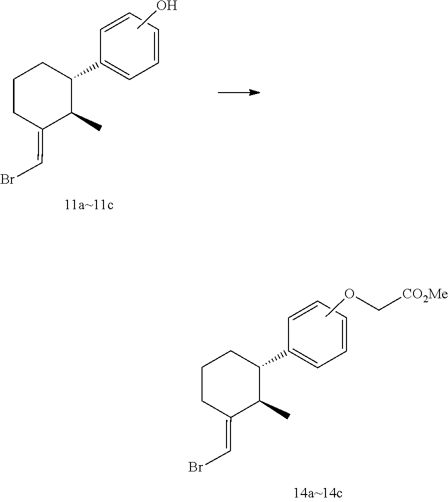

### Synthesis Example 8-1

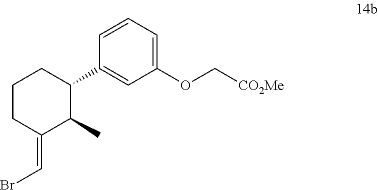 [SMILES: COC(=O)COc1cccc([C@H]2CCC/C(=C\Br)[C@@H]2C)c1]

Anhydrous potassium carbonate (28 mg, 2.0 mmol, 4.0 equivalents) and methyl 2-iodoacetate (24 mg, 2.0 mmol, 4.0 equivalents) were added to a solution of compound 11 b (0.14 g, 0.5 mmol, 1.0 equivalent) in N,N-dimethylformamide (1.0 mL). The mixture was stirred at 50° C. for 24 hours. After the reaction mixture was cooled to room temperature, water was added to the reaction mixture, followed by extraction with diethyl ether. The resulting organic layer was washed with a saturated aqueous sodium chloride solution, then dried over anhydrous magnesium sulfate, and then filtered, and the filtrate was concentrated. The resulting residue was purified by silica gel column chromatography (hexane/ethyl acetate) to give compound 14b (0.14 g) in a yield of 80%.

¹H NMR (600 MHz, CDCl₃) δ 7.22 (t, J=7.8 Hz, 1H, Ar), 6.81 (d, J=7.8 Hz, 1H, Ar), 6.73 (s, 1H, Ar), 6.72 (dd, J=2.4, 8.4 Hz, 1H, Ar), 5.93 (s, 1H, C═CHBr), 4.63 (s, 2H, CH₂C(O)OCH₃), 3.81 (s, 3H, CH₂C(O)OCH₃), 3.10 (brd, J=12.9 Hz, 1H, CHAr), 2.32 (dq, J=10.8, 6.6 Hz, 1H, CHCH₃), 2.20 (dt, J=3.6, 12.0 Hz, 1H, CH₂), 1.96-1.87 (m, 3H, CH₂), 1.67 (dq, J=3.0, 12.6 Hz, 1H, CH₂), 1.43 (tq, J=3.0, 12.6, 1H, CH₂), 0.82 (d, J=6.6 Hz, 3H, CHCH₃).

¹³C NMR (150 MHz, CDCl₃) δ 169.4, 157.9, 148.2, 147.2, 129.5, 121.0, 114.3, 111.7, 99.1, 65.4, 53.3, 52.3, 43.7, 35.3, 32.4, 26.6, 15.7.
IR (ATR) 2954, 2929, 2877, 2855, 1763, 1740, 1608, 1585, 1486, 1439, 1376, 1289, 1207, 1159, 1089, 879, 857, 786, 700 cm⁻¹.
HR-MS m/z=calcd for C₁₇H₂₁BrNaO₃ [M+Na]⁺: 375.05718, found 375.05864.

### Synthesis Example 8-2

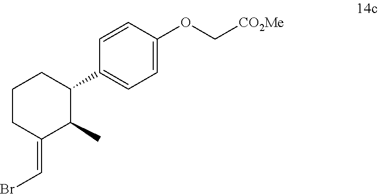 [SMILES: COC(=O)COc1ccc([C@H]2CCC/C(=C\Br)[C@@H]2C)cc1]

Similar to the synthesis of compound 14b from compound 11b, compound 14c (0.17 g) was synthesized in a yield of 96% from compound 11c (0.14 g, 0.5 mmol) and methyl 2-iodoacetate (24 mg, 2.0 mmol).

¹H NMR (600 MHz, CDCl₃) δ 7.08 (d, J=8.4 Hz, 2H, Ar), 6.85 (d, J=8.4 Hz, 2H, Ar), 5.92 (s, 1H, C═CHBr), 4.62 (s, 2H, CH₂C(O)OCH₃), 3.81 (s, 3H, CH₂C(O)OCH₃), 3.09 (brd, J=12.9 Hz, 1H, CHAr), 2.28 (dq, J=12.0, 6.6 Hz, 1H, CHCH₃), 2.19 (dt, J=3.6, 11.4 Hz, 1H, CH₂), 1.94-1.83 (m, 3H, CH₂), 1.65 (dq, J=3.6, 12.6 Hz, 1H, CH₂), 1.42 (tq, J=3.6, 12.6 Hz, 1H, CH₂), 0.80 (d, J=6.6 Hz, 3H, CHCH₃).

¹³C NMR (150 MHz, CDCl₃) δ 169.5, 156.2, 148.4, 138.7, 128.3, 114.6, 99.0, 65.5, 52.5, 52.2, 44.0, 35.5, 32.4, 26.6, 15.7.
IR (ATR) 2954, 2929, 2873, 2855, 1762, 1740, 1609, 1586, 1510, 1438, 1376, 1308, 1288, 1203, 1177, 1085, 849, 828, 801, 705 cm⁻¹.
HR-MS m/z=calcd for C₁₇H₂₁BrNaO₃ [M+Na]⁺: 375.05718, found 375.05975.

### Synthesis Example 9

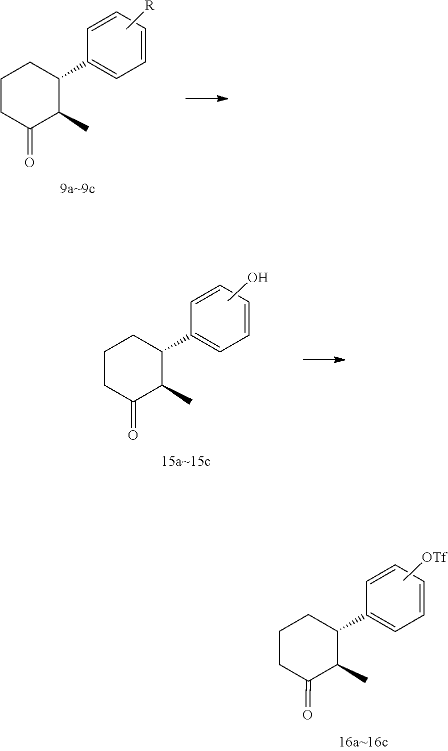

### Synthesis Example 9-1

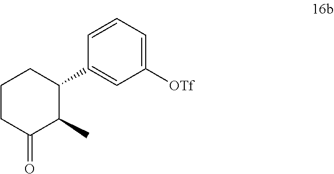 [SMILES: C[C@H]1C(=O)CCC[C@@H]1c1cccc(OS(=O)(=O)C(F)(F)F)c1]

To a solution of compound 9b (0.94 g, 3.8 mmol, 1.0 equivalent) in methanol (8.0 mL) was added 3 M hydrochloric acid (4.0 mL) at 0° C. After the mixture was stirred for 24 hours, the reaction was quenched by adding a saturated aqueous sodium bicarbonate solution, followed by extraction with diethyl ether. The resulting organic layer was washed with a saturated aqueous sodium chloride solution, then dried over anhydrous sodium sulfate, and then filtered. The filtrate was concentrated under reduced pressure to give a crude product of compound 15b.

Trifluoromethanesulfonic anhydride (0.94 mL, 5.7 mmol, 1.5 equivalents) was added at 0° C. to a solution of the crude product of compound 15b and triethylamine (1.1 mL, 7.6 mmol, 2.0 equivalents) in methylene chloride (7.6 mL). After the mixture was stirred at room temperature for 1 hour, the reaction was quenched by adding water, followed by extraction with ethyl acetate. The resulting organic layer was washed with a saturated aqueous sodium chloride solution, then dried over anhydrous sodium sulfate, and then filtered, and the filtrate was concentrated. The resulting residue was purified by silica gel column chromatography (hexane/ethyl acetate) to give compound 16b (1.26 g) in a yield of 93%.

¹H NMR (600 MHz, CDCl₃) δ 7.42 (t, J=7.8 Hz, 1H, Ar), 7.24 (d, J=7.8 Hz, 1H, Ar), 7.16 (dd, J=2.4, 8.4 Hz, 1H, Ar), 7.12 (s, 1H, Ar), 2.64-2.56 (m, 2H), 2.53 (brd, J=13.8 Hz, 1H), 2.48 (dt, J=6.0, 13.2 Hz, 1H), 2.21-2.16 (m, 1H), 2.02 (brd, J=12.0 Hz, 1H), 1.93 (dq, J=3.6, 12.0 Hz, 1H), 1.78 (tq, J=4.2 13.2 Hz, 1H), 0.82 (d, J=6.0 Hz, 3H, CHCH₃).

¹³C NMR (150 MHz, CDCl₃) δ 211.1, 149.8, 146.9, 130.5, 127.3, 120.0, 119.5, 117.7, 52.7, 50.2, 41.6, 34.3, 26.2, 12.1.
IR (ATR) 2970, 2936, 2873, 1711, 1613, 1580, 1446, 1419, 1247, 1207, 1139, 1119, 968, 904, 850, 818, 793, 734, 695, 653 cm⁻¹.
HR-MS m/z=calcd for C₁₄H₁₅F₃NaO₄S [M+Na]⁺: 359.05408, found 359.05860.

### Synthesis Example 10

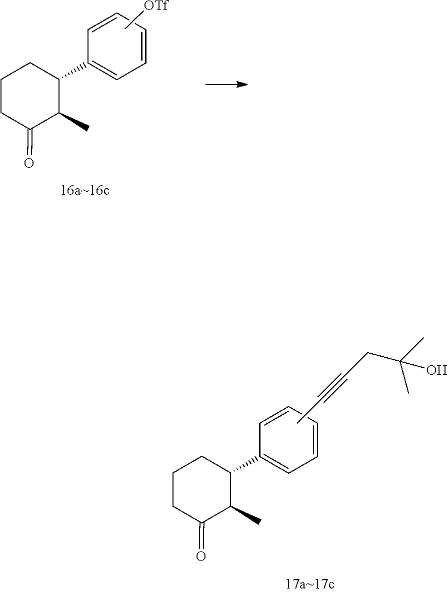

### Synthesis Example 10-1

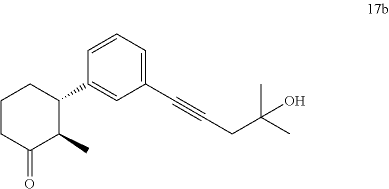 [SMILES: C[C@H]1C(=O)CCC[C@@H]1c1cccc(C#CCC(C)(C)O)c1]

A solution of compound 16b (0.94 g, 1.0 equivalent), 2-methylpent-4-yn-2-ol (2.75 g, 28 mmol, 10.0 equivalents), and triethylamine (3.9 mL, 28.0 mmol, 10.0 equivalents) in N,N dimethylformamide (5.6 mL) was added to a mixture of copper iodide (27 mg, 0.14 mmol, 5.0 mol %) and bis(triphenylphosphine)palladium(II) dichloride (0.10 g, 0.14 mmol, 5.0 mol %). After the mixture was stirred at 60° C. for 18 hours, the reaction solution was concentrated. The resulting concentrate was purified by silica gel column chromatography (hexane/ethyl acetate) to give compound 17b (0.73 g) in a yield of 92%.

¹H NMR (600 MHz, CDCl₃) δ 7.30 (d, J=7.8 Hz, 1H, Ar), 7.28 (s, 1H, Ar), 7.26 (t, J=7.8 Hz, 1H, Ar), 7.13 (d, J=7.2 Hz, 1H, Ar), 2.64-2.58 (m, 1H), 2.61 (s, 2H, C(triple bond)CCH₂), 2.53 (dd, J=4.2, 11.4 Hz, 1H), 2.53-2.47 (m, 1H), 2.46 (dd, J=6.0, 13.2 Hz, 1H), 2.18-2.13 (m, 1H), 2.00-1.93 (m, 2H), 1.93 (dq, J=3.0, 12.6 Hz, 1H), 1.76 (tq, J=4.2, 13.2 Hz, 1H), 1.39 (s, 6H, C(CH₃)₂), 0.81 (d, J=6.0 Hz, 3H, CHCH₃).

¹³C NMR (150 MHz, CDCl₃) δ 212.0, 144.0, 130.3, 130.0, 128.6, 127.0, 123.7, 86.4, 83.4, 70.2, 52.9, 50.3, 41.8, 35.1, 34.4, 28.8, 26.4, 12.3.
IR (ATR) 3429, 2971, 2932, 2871, 2227, 1705, 1599, 1579, 1482, 1448, 1427, 1377, 1361, 1246, 1218, 1138, 1020, 973, 905, 797, 699 cm⁻¹.
HR-MS m/z=calcd. for C₁₉H₂₄NaO [M+Na]⁺: 307.16740, found 307.16691.

### Synthesis Example 11

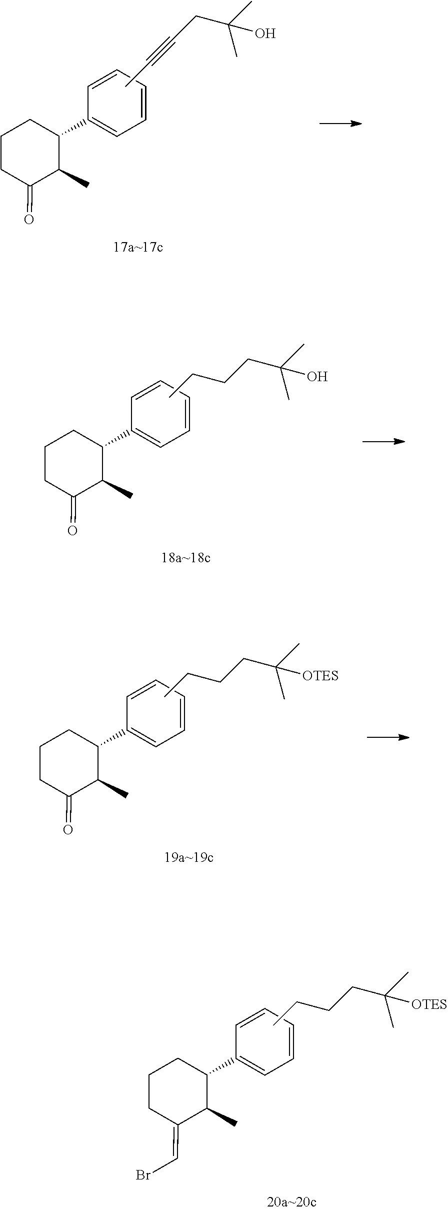

### Synthesis Example 11-1

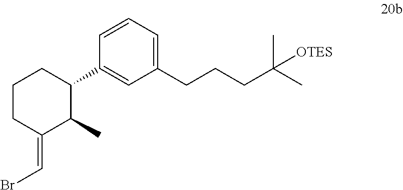

Palladium (supported on charcoal, 50.5 mg) was added in an amount of 10 mass % to a solution of compound 17b (1.1 g, 3.8 mmol, 1.0 equivalent) in ethyl acetate (38 mL). Under a hydrogen atmosphere, the mixture was stirred at room temperature for 24 hours. The reaction mixture was filtered through Celite. The filtrate was concentrated to give a crude product of compound 18b.

Chlorotriethylsilane (0.95 mL, 5.7 mmol, 1.5 equivalents) was added at 0° C. to a solution of the crude product of compound 18b and imidazole (0.52 g, 7.6 mmol, 2.0 equivalents) in N,N-dimethylformamide (7.6 mL). After the mixture was stirred at room temperature for 12 hours, a saturated aqueous sodium bicarbonate solution was added at 0° C. to the reaction mixture, followed by extraction with hexane. The organic layer was dried over anhydrous magnesium sulfate and then filtered. The filtrate was concentrated to give a crude product of compound 19b.

A 0.5 M tetrahydrofuran solution of lithium diisopropylamide (15.2 mL, 7.6 mmol, 2.0 equivalents) was added at −78° C. to a mixture of (bromomethyl)triphenylphosphonium bromide (3.3 g, 7.6 mmol, 2.0 equivalents) and tetrahydrofuran (7.6 mL). The mixture was heated to 0° C. and stirred for 1 hour. After the resulting mixture was cooled again to −78° C., a solution of the crude product of compound 19b in tetrahydrofuran (7.6 mL) was added to the mixture and stirred for 12 hours with heating at 0° C. The reaction mixture was filtered through silica gel with hexane. The resulting filtrate was concentrated under reduced pressure. The concentrate was purified by silica gel column chromatography (hexane/ethyl acetate) to give compound 20b (1.0 g) in a yield of 76%.

¹H NMR (600 MHz, CDCl₃) δ 7.20 (t, J=7.2 Hz, 1H, Ar), 7.02 (d, J=7.2 Hz, 1H, Ar), 6.97 (d, J=7.2 Hz, 1H, Ar), 6.96 (s, 1H, Ar), 5.93 (s, 1H, C═CHBr), 3.13-3.08 (m, 1H, CHAr), 2.57 (t, J=7.8 Hz, 2H, ArCH₂CH₂CH₂), 2.35 (dq, J=11.4, 6.6 Hz, 1H, CHCH₃), 2.20 (dt, J=3.6, 11.4 Hz, 1H), 1.96-1.86 (m, 3H), 1.73-1.64 (m, 3H), 1.47-1.42 (m, 3H), 1.18 (s, 6H, C(CH₃)₂), 0.93 (t, J=8.1 Hz, 9H, Si(CH₂CH₃)₃), 0.81 (d, J=6.6 Hz, 3H, CHCH₃), 0.54 (q, J=8.1 Hz, 6H, Si(CH₂CH₃)₃).

¹³C NMR (150 MHz, CDCl₃) δ 148.5, 145.1, 143.0, 128.3, 127.5, 126.4, 124.6, 98.9, 73.3, 53.4, 44.6, 43.8, 36.4, 35.4, 32.5, 29.9, 26.7, 26.3, 15.7, 7.1, 6.8.
IR (ATR) 2932, 2912, 2873, 1606, 1458, 1444, 1378, 1363, 1234, 1201, 1151, 1126, 1045, 1031, 1015, 889, 791, 741, 721, 705, 670 cm⁻¹.

### Synthesis Example 12

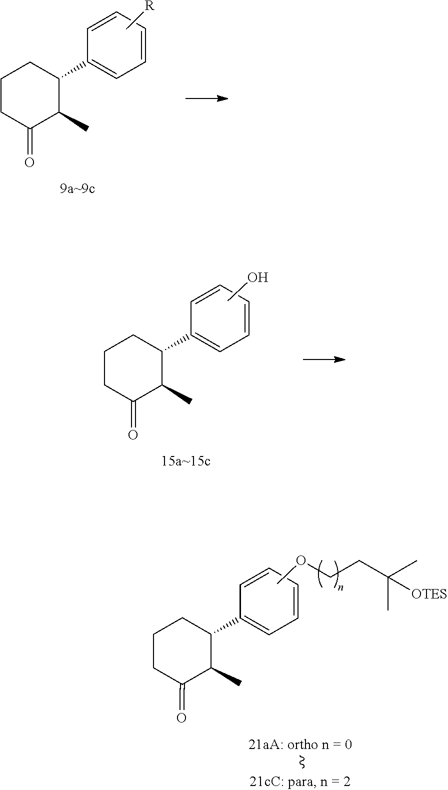

### Synthesis Example 12-1

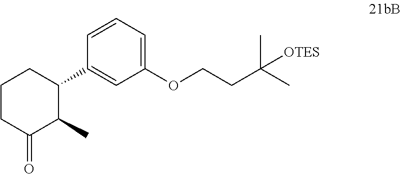

At 0° C., 3 M hydrochloric acid (0.5 mL) was added to a solution of compound 9b (0.12 g, 0.5 mmol, 1.0 equivalent) in methanol (1.0 mL). After the mixture was stirred for 24 hours, the reaction was quenched by adding a saturated aqueous sodium bicarbonate solution, followed by extraction with diethyl ether. The resulting organic layer was washed with a saturated aqueous sodium chloride solution, then dried over anhydrous sodium sulfate, and then filtered. The filtrate was concentrated under reduced pressure to give a crude product of compound 15b.

Anhydrous potassium carbonate (0.28 g, 4.0 mmol, 4.0 equivalents) and triethyl((4-iodo-2-methylbutan-2-yl)oxy)silane (13B) (0.66 g, 2.0 mmol, 4.0 equivalents) were added to a solution of the crude product of compound 15b in N,N-dimethylformamide (1.0 mL). The mixture was stirred at 50° C. for 24 hours. After the reaction mixture was cooled to room temperature, water was added to the reaction mixture, followed by extraction with hexane. The resulting organic layer was washed with a saturated aqueous sodium chloride solution, then dried over anhydrous magnesium sulfate, and then filtered, and the filtrate was concentrated. The resulting residue was purified by silica gel column chromatography (hexane/ethyl acetate) to give compound 21bB (0.20 g) in a yield of 98%.

¹H NMR (600 MHz, CDCl₃) δ 7.23 (t, J=7.8 Hz, 1H, Ar), 6.77 (d, J=7.8 Hz, 2H, Ar), 6.74 (s, 1H, Ar), 4.12 (t, J=7.2 Hz, 2H, OCH₂), 2.61 (dq, J=12.0, 6.6 Hz, 1H, CHCH₃), 2.52 (dd, J=3.6, 12.0 Hz, 1H), 2.51-2.47 (m, 1H), 2.45 (dd, J=6.0, 13.2 Hz, 1H), 2.17-2.13 (m, 1H), 2.02-1.89 (m, 2H), 1.95 (dd, J=7.2 Hz, 2H, OCH₂CH₂), 1.75 (tq, J=4.2, 13.2 Hz, 1H), 1.31 (s, 6H, C(CH₃)₂), 0.95 (t, J=7.8 Hz, 9H, Si(CH₂CH₃)₃), 0.82 (d, J=6.6 Hz, 3H, CHCH₃), 0.59 (q, J=7.8 Hz, 6H, Si(CH₂CH₃)₃).

¹³C NMR (150 MHz, CDCl₃) δ 212.3, 159.3, 145.5, 129.5, 119.3, 113.8, 112.0, 72.4, 64.7, 53.2, 50.5, 43.6, 41.8, 34.4, 30.4, 26.5, 12.2, 7.1, 6.7.
IR (ATR) 2954, 2934, 2912, 2874, 1711, 1600, 1583, 1488, 1447, 1381, 1365, 1284, 1266, 1240, 1219, 1156, 1078, 1050, 1029, 1017, 961, 849, 785, 742, 721, 698, 671 cm⁻¹.
HR-MS m/z=calcd for C₂₄H₄₀NaO₃Si [M+Na]⁺: 427.26444, found 427.26627.

### Synthesis Example 13

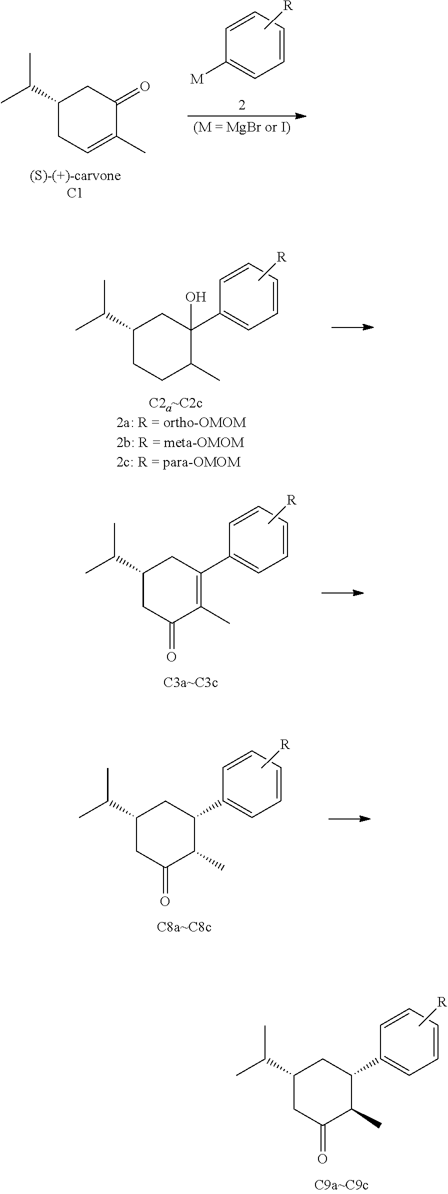

### Synthesis Example 13-1

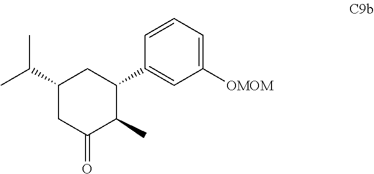

Under an argon atmosphere at −78° C., a 1.59 M hexane solution of n-butyllithium (5.0 mL, 7.8 mmol, 1.5 equivalents) was added dropwise to a mixture of 1-bromo-3-(methoxymethoxy)benzene (1.63 g, 7.5 mmol, 1.5 equivalents) and THF (11.2 mL). The mixture was stirred for 1 hour to form an alkyl lithium reagent. At −78° C., (S)-(+)-carvone (C1) (0.75 g, 5.0 mmol, 1.0 equivalent) was added to the alkyl lithium reagent. The resulting mixture was heated to room temperature and stirred for 2 hours. The reaction was quenched by adding a saturated ammonium chloride solution, followed by extraction with ethyl acetate. The resulting organic layer was washed with a saturated aqueous sodium chloride solution, then dried over anhydrous magnesium sulfate, and then filtered through Celite. The filtrate was concentrated under reduced pressure to give a crude product of compound C2b.

Under an argon atmosphere, a solution of the crude product of compound C2b in methylene chloride (7.1 mL) was added at 0° C. to a methylene chloride solution (21 mL) containing pyridinium chlorochromate (1.6 g, 7.5 mmol, 1.5 equivalents) and 4 Å molecular sieves (0.5 g). After the mixture was stirred at room temperature for 3 hours, the solid was filtered off, and the filtrate was concentrated to give a crude product of compound C3a.

Palladium (supported on charcoal, 72 mg) was added in an amount of 10 mass % to a solution of the crude product of compound C3a in ethyl acetate (50 mL). Under a hydrogen atmosphere, the mixture was stirred at room temperature for 24 hours. After the reaction mixture was filtered through Celite, the filtrate was concentrated to give a crude product of compound C8b.

Potassium carbonate (2.0 g, 15 mmol, 3.0 equivalents) was added to a solution of the crude product of compound C8b in methanol (10 mL). After the mixture was stirred at room temperature for 24 hours, water was added to the mixture. After the reaction mixture was extracted with hexane, the organic layer was dried over anhydrous magnesium sulfate. After the resulting organic layer was filtrated, the filtrate was concentrated. The resulting residue was purified by silica gel column chromatography to give compound C9b (0.53 g) in a yield of 37%.

¹H NMR (600 MHz, CDCl₃) δ 7.25 (t, J=7.8 Hz, 1H, Ar), 6.93 (dd, J=2.4, 7.8 Hz, 1H, Ar), 6.87 (d, J=2.4 Hz, 1H, Ar), 6.85 (d, J=7.8 Hz, 1H, Ar), 5.19 (d, J=6.6 Hz, 1H, OCH₂O), 5.17 (d, J=6.6 Hz, 1H, OCH₂O), 3.50 (s, 3H, OCH₃), 2.57 (dq, J=12.6, 6.6 Hz, 1H, CHCH₃), 2.50 (dt, J=12.6, 2.4 Hz, 1H, CHAr), 2.47 (dt, J=3.6, 12.0 Hz, 1H), 2.26-2.19 (m, 1H), 1.99 (dt, J=10.8, 2.4 Hz, 1H), 1.71-1.64 (m, 2H), 1.59 (dq, J=13.2, 6.6 Hz, 1H), 0.91 (d, J=6.6 Hz, 3H, CH(CH₃)₂), 0.90 (d, J=6.6 Hz, 3H, CH(CH₃)₂), 0.82 (d, J=6.6 Hz, 3H, CHCH₃).

¹³C NMR (150 MHz, CDCl₃) δ 212.5, 157.6, 145.7, 129.6, 120.7, 115.3, 114.0, 94.5, 56.0, 51.9, 49.9, 45.2, 44.8, 38.2, 32.7, 19.5, 19.3, 12.1.
IR (ATR) 2958, 2932, 2873, 2826, 1708, 1584, 1486, 1450, 1370, 1313, 1252, 1216, 1149, 1078, 1010, 994, 941, 922, 872, 779, 700 cm⁻¹.
HR-MS m/z=calcd for C₁₈H₂₆NaO₃ [M+Na]⁺: 313.17796, found 313.17905.

### Synthesis Example 14

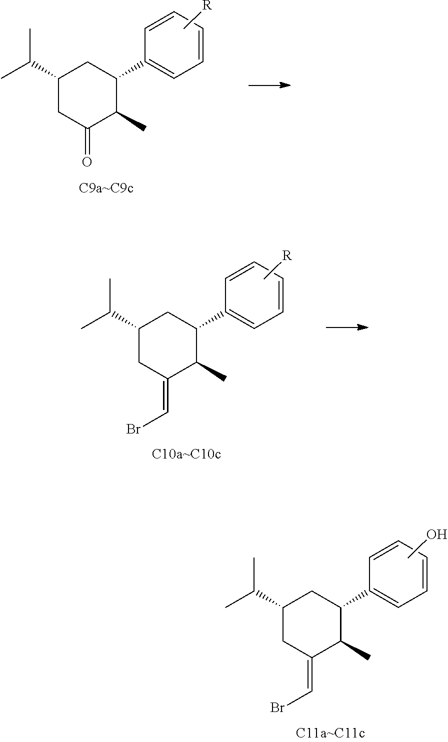

### Synthesis Example 14-1

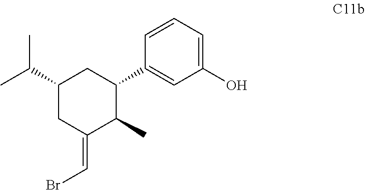 [SMILES: CC(C)[C@H]1C/C(=C\Br)[C@H](C)[C@@H](c2cccc(O)c2)C1]

A 0.5 M THF solution of lithium diisopropylamide (5.0 mL, 2.5 mmol, 2.0 equivalents) was added at −78° C. to a mixture of (bromomethyl)triphenylphosphonium bromide (1.1 g, 2.5 mmol, 2.0 equivalents) and THF (2.5 mL). The mixture was heated to 0° C. and stirred for 1 hour. After the mixture was cooled again to −78° C., a solution of compound C9b (0.31 g, 1.25 mmol, 1.0 eq.) in tetrahydrofuran (3.0 mL) was added to the mixture and stirred for 12 while being heated at 0° C. The reaction mixture was filtered through silica gel with hexane. The resulting filtrate was concentrated under reduced pressure to give a crude product of compound C10b.

At 0° C., 3 M hydrochloric acid (1.25 mL) was added to a solution of the crude product of compound C10b in methanol (2.5 mL). After the mixture was stirred for 24 hours, the reaction was quenched by adding a saturated aqueous sodium bicarbonate solution, followed by extraction with diethyl ether. The resulting organic layer was washed with a saturated aqueous sodium chloride solution, then dried over anhydrous sodium sulfate, and then filtered, and the filtrate was concentrated. The resulting residue was purified by silica gel column chromatography (hexane/ethyl acetate) to give compound C11b (0.31 g) in a yield of 76%.

¹H NMR (600 MHz, CDCl₃) δ 7.17 (t, J=7.5 Hz, 1H, Ar), 6.75 (d, J=7.2 Hz, 1H, Ar), 6.67 (dd, J=1,8, 7.8 Hz, 1H, Ar), 6.66 (s, 1H, Ar), 5.92 (s, 1H, C═CHBr), 4.63 (s, 1H, OH), 3.12 (dt, J=12.6, 2.4 Hz, 1H, CHAr), 2.27 (dq, J=11.4, 6.0 Hz, 1H, CHCH₃), 2.16 (dt, J=3.0, 11.4 Hz, 1H, CH₂), 1.87 (dq, J=13.2, 2.4 Hz, 1H), 1.61 (t, J=12.6 Hz, 1H), 1.8-1.52 (m, 1H), 1.40 (q, J=12.6 Hz, 1H), 1.34-1.27 (m, 1H), 0.94 (d, J=7.2 Hz, 3H, CH(CH₃)₂), 0.91 (d, J=7.2 Hz, 3H, CH(CH₃)₂), 0.83 (d, J=6.6 Hz, 3H, CHCH₃).

¹³C NMR (150 MHz, CDCl₃) δ 155.6, 148.3, 147.4, 129.7, 119.9, 114.1, 113.2, 99.1, 53.0, 44.5, 43.6, 38.8, 36.0, 32.6, 19.7, 19.5, 15.4.
IR (ATR) 3347, 2957, 2932, 2912, 2872, 1704, 1612, 1589, 1484, 1454, 1368, 1331, 1262, 1234, 1155, 970, 886, 864, 842, 783, 757, 730, 699 cm⁻¹.
HR-MS m/z=calcd for C₁₇H₂₂BrO [M−H]⁻: 321.08540, found 321.08745.

### Synthesis Example 15

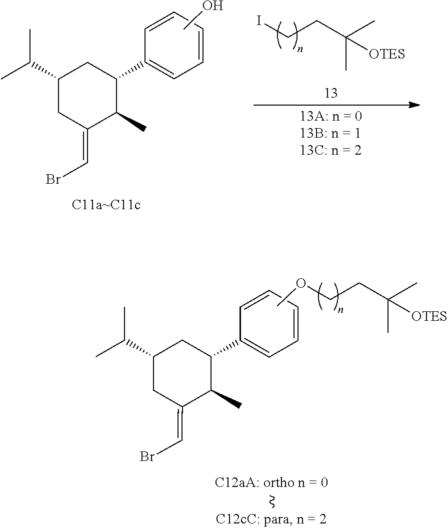

### Synthesis Example 15-1

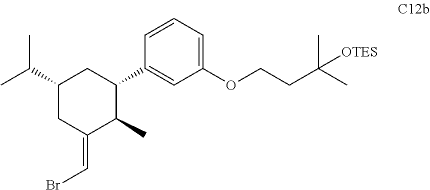

Anhydrous potassium carbonate (1.45 g, 10.5 mmol, 10.0 equivalents) and triethyl((4-iodo-2-methylbutan-2-yl)oxy)silane (13B) (1.72 g, 5.25 mmol, 5.0 equivalents) were added to a solution of compound C11b (0.34 g, 1.05 mmol, 1.0 equivalent) in N,N-dimethylformamide (3 mL). The mixture was stirred at 50° C. for 24 hours. After the reaction mixture was cooled to room temperature, water was added to the reaction mixture, followed by extraction with hexane. The resulting organic layer was washed with a saturated aqueous sodium chloride solution, then dried over anhydrous magnesium sulfate, and then filtered, and the filtrate was concentrated. The resulting residue was purified by silica gel column chromatography (hexane/ethyl acetate) to give compound C12b (0.52 g) in a yield of 94%.

¹H NMR (600 MHz, CDCl₃) δ 7.22 (t, J=7.8 Hz, 1H, Ar), 6.75 (d, J=7.8 Hz, 1H, Ar), 6.74 (d, J=7.8 Hz, 1H, Ar), 6.71 (s, 1H, Ar), 5.92 (s, 1H, C═CHBr), 4.12 (t, J=7.2 Hz, 2H, OCH₂), 3.12 (dt, J=13.2, 2.4 Hz, 1H, CHAr), 2.28 (dq, J=11.4, 6.6 Hz, 1H, CHCH₃), 2.17 (dt, J=3.0, 12.0 Hz, 1H), 1.94 (t, J=7.2 Hz, 2H, OCH₂CH₂), 1.88 (brd, J=13.2 Hz, 1H), 1.61 (t, J=12.6 Hz, 1H), 1.59-1.52 (m, 1H), 1.41 (q, J=12.6 Hz, 1H), 1.33-1.28 (m, 1H), 1.30 (s, 6H, C(CH₃)₂), 0.95 (t, J=7.8 Hz, 9H, Si(CH₂CH₃)₃), 0.94 (d, J=7.2 Hz, 3H, CH(CH₃)₂), 0.90 (d, J=7.2 Hz, 3H, CH(CH₃)₂), 0.82 (d, J=6.0 Hz, 3H, CHCH₃), 0.59 (q, J=7.8 Hz, 6H, Si(CH₂CH₃)₃).

¹³C NMR (150 MHz, CDCl₃) δ 159.2, 148.4, 147.0, 129.4, 119.6, 113.9, 111.7, 99.0, 72.4, 64.7, 53.2, 44.5, 43.7, 38.8, 36.0, 32.7, 30.4, 19.8, 19.5, 15.4, 7.1, 6.7.
IR (ATR) 2955, 2934, 2911, 2874, 1730, 1600, 1583, 1457, 1382, 1365, 1289, 1263, 1222, 1157, 1031, 1016, 870, 784, 725, 700 cm⁻¹.

### Synthesis Example 16

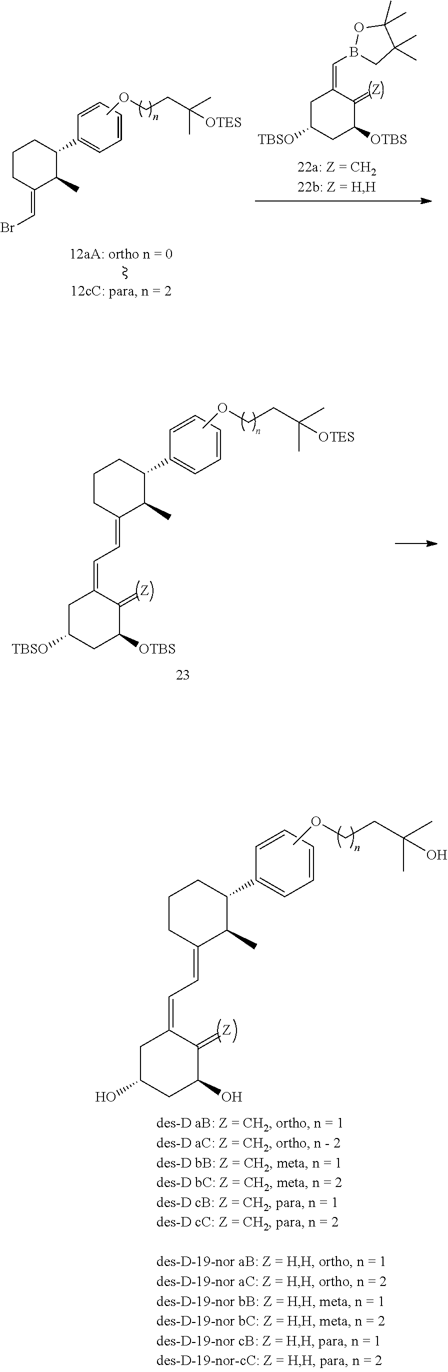

### Synthesis Example 16-1

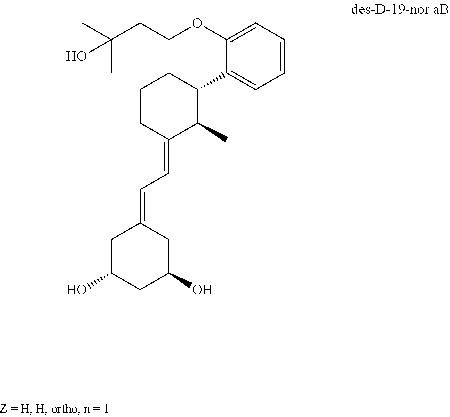 [SMILES: C[C@H]1/C(=C/C=C2C[C@@H](O)C[C@H](O)C2)CCC[C@@H]1c1ccccc1OCCC(C)(C)O]

A solution of compound 12aB (48.2 mg, 0.1 mmol, 1.0 equivalent) and compound 22b (53 mg, 0.11 mmol, 1.1 equivalents) in tetrahydrofuran (1.0 mL) and a 3 N potassium hydroxide solution (33 μL) were added to 1,1′-bis(diphenylphosphino)ferrocene-palladium(II) dichloride (5.9 mg, 8.0 μmol, 8.0 mol %). The mixture was stirred at 50° C. for 12 hours. After being cooled to room temperature, the reaction solution was dried over anhydrous sodium sulfate and then filtered through Celite with diethyl ether. The resulting filtrate was concentrated under reduced pressure to give a crude product of compound 23.

A 1.0 M tetrahydrofuran solution of tetrabutylammonium fluoride (0.6 mL, 0.6 mmol, 6.0 equivalents) was added at 0° C. to a solution of the crude product of compound 23 in tetrahydrofuran (0.1 mL). After the mixture was stirred at room temperature for 12 hours, the reaction was quenched with a saturated ammonium chloride solution, followed by extraction with diethyl ether. The resulting organic layer was dried over anhydrous magnesium sulfate and filtered, and the filtrate was concentrated. The resulting residue was purified by silica gel column chromatography (hexane/ethyl acetate/methanol) to give compound des-D-19-nor aB (24.5 mg) in a yield of 59%.

¹H NMR (600 MHz, CDCl₃) δ 7.24 (d, J=8.4 Hz, 1H, Ar), 7.16 (dd, J=8.4, 7.8 Hz, 1H, Ar), 6.96 (t, J=7.8 Hz, 1H, Ar), 6.89 (d, J=7.8 Hz, 1H, Ar), 6.33 (d, J=10.8 Hz, 1H, CH═C), 6.08 (d, J=10.8 Hz, 1H, CH═C), 4.19-4.09 (m, 4H, CHO, OCH₂), 2.95 (brd, J=13.2 Hz, 1H, CHAr), 2.76-2.69 (m, 1H), 2.57 (brd, J=13.2 Hz, 1H), 2.54 (dd, J=3.0, 13.2 Hz, 1H), 2.47 (dd, J=3.6, 13.2 Hz, 1H), 2.43 (dd, J=7.2, 13.2 Hz, 1H), 2.37 (brs, 1H), 2.25-2.14 (m, 2H), 2.05-1.96 (m, 2H), 1.95-1.61 (m, 7H), 1.47-1.37 (m, 1H), 1.31 (s, 6H, C(CH₃)₂), 0.84 (d, J=6.6 Hz, 3H, CHCH₃).

¹³C NMR (150 MHz, CDCl₃) δ 156.0, 146.4, 142.0, 134.3, 132.6, 126.6, 123.8, 121.1, 114.6, 111.4, 70.6, 67.5, 67.1 (2C), 65.4, 45.2, 43.0, 42.0, 41.8, 36.9, 30.0, 29.6 (2C), 29.3, 27.7, 15.5.
IR (ATR) 3349, 2965, 2927, 2885, 2853, 1598, 1492, 1449, 1365, 1289, 1228, 1158, 1088, 1049, 974, 940, 885, 813, 752 cm⁻¹.
HR-MS m/z=calcd for C₂₆H₃₈NaO₄ [M+Na]⁺: 437.26678, found 437.26733.

### Synthesis Example 16-2

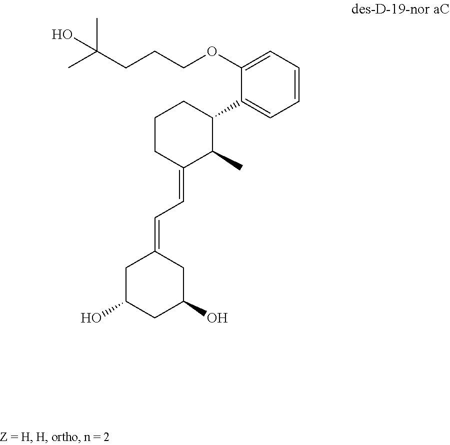 [SMILES: C[C@H]1/C(=C/C=C2C[C@@H](O)C[C@H](O)C2)CCC[C@@H]1c1ccccc1OCCCC(C)(C)O]

Similar to the synthesis of compound des-D-19-nor aB from compound 12aB, compound des-D-19-nor aC (30.0 mg) was synthesized in a yield of 71% from compound 12aC (49.6 mg, 0.1 mmol) and compound 22b (53 mg, 0.11 mmol).

¹H NMR (600 MHz, CDCl₃) δ 7.20 (d, J=7.2 Hz, 1H, Ar), 7.14 (dd, J=7.2, 8.4 Hz, 1H, Ar), 6.92 (t, J=7.2 Hz, 1H, Ar), 6.83 (d, J=8.4 Hz, 1H, Ar), 6.36 (d, J=10.8 Hz, 1H, CH═C), 6.09 (d, J=10.8 Hz, 1H, CH═C), 4.11 (brs, 2H, OCH₂), 3.99-3.91 (m, 2H, CHO), 2.97 (brd, J=13.2 Hz, 1H, CHAr), 2.83 (brs, 1H), 2.62 (dd, J=3.0, 13.2 Hz, 1H), 2.54 (dd, J=3.0, 13.2 Hz, 1H), 2.48 (dd, J=3.6, 13.2 Hz, 1H), 2.39 (dd, J=7.2, 13.2 Hz, 1H), 2.45-2.37 (m, 1H), 2.21 (dd, J=7.2, 13.2 Hz, 1H), 2.16 (dd, J=7.2, 13.2 Hz, 1H), 2.01 (brs, 1H), 1.97-1.58 (m, 10H), 1.47-1.36 (m, 1H), 1.25 (s, 6H, C(CH₃)₂), 0.85 (d, J=6.0 Hz, 3H, CHCH₃).

¹³C NMR (150 MHz, CDCl₃) δ 156.5, 147.0, 134.5, 132.3, 126.6, 124.0, 120.7, 114.3, 113.4, 111.6, 70.8, 68.4, 67.4, 67.2, 45.1, 43.0, 42.1, 40.6, 36.8, 34.1, 30.1 (2C), 29.2, 29.1, 27.8, 24.7, 15.6.
IR (ATR) 3353, 2963, 2927, 2881, 2854, 1599, 1492, 1450, 1365, 1289, 1234, 1157, 1127, 1087, 1046, 973, 939, 886, 814, 752 cm⁻¹.
HR-MS m/z=calcd for C₂₇H₄₀NaO₄ [M+Na]⁺: 451.28243, found 451.28674.

### Synthesis Example 16-3

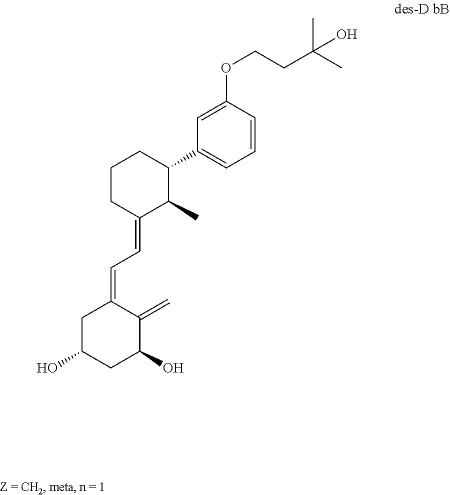 [SMILES: C=C1/C(=C\C=C2/CCC[C@H](c3cccc(OCCC(C)(C)O)c3)[C@H]2C)C[C@@H](O)C[C@@H]1O]

Similar to the synthesis of compound des-D-19-nor aB from compound 12aB, compound des-D bB (16.7 mg) was synthesized in a yield of 40% from compound 12bB (48 mg, 0.1 mmol) and compound 22a (55 mg, 0.11 mmol).

¹H NMR (600 MHz, CDCl₃) δ 7.20 (dd, J=7.2, 8.4 Hz, 1H, Ar), 6.79 (d, J=7.2 Hz, 1H, Ar), 6.75-6.74 (m, 2H, Ar), 6.41 (d, J=10.8 Hz, 1H, CH═C), 6.25 (d, J=10.8 Hz, 1H, CH═C), 5.34 (s, 1H, C═CH₂), 5.04 (s, 1H, C═CH₂), 4.45 (brt, 1H, CHO), 4.24 (brs, 1H, CHO), 4.18 (t, J=6.0 Hz, 2H, OCH₂), 2.96 (brd, J=13.2 Hz, 1H, CHAr), 2.63 (dd, J=3.0, 13.2 Hz, 1H), 2.40 (brs, 1H), 2.34 (dd, J=7.2, 13.2 Hz, 1H), 2.35-2.27 (m, 1H), 2.21 (dt, J=3.0, 10.8 Hz, 1H, CHAr), 2.00 (t, J=6.0 Hz, 2H, OCH₂CH₂), 2.02-1.97 (m, 1H), 1.95-1.87 (m, 3H), 1.72-1.57 (m, 3H), 1.46-1.38 (m, 1H), 1.32 (s, 6H, C(CH₃)₂O), 0.80 (d, J=6.0 Hz, 3H, CHCH₃).

¹³C NMR (150 MHz, CDCl₃) δ 148.1, 147.5, 146.2, 134.1, 129.3, 125.2, 120.3, 116.6, 113.9, 112.3, 111.5, 71.1, 70.5, 66.8, 65.1, 53.6, 45.5, 43.6, 42.8, 41.6, 35.4, 29.8, 29.6, 27.4, 16.1.
IR (ATR) 3357, 2965, 2927, 2877, 1716, 1698, 1653, 1600, 1583, 1446, 1377, 1263, 1243, 1157, 1038, 911, 882, 784, 754, 701 cm⁻¹.
HR-MS m/z=calcd for C₂₇H₃₈NaO₄ [M+Na]⁺: 449.26678, found 449.26855.

### Synthesis Example 16-4

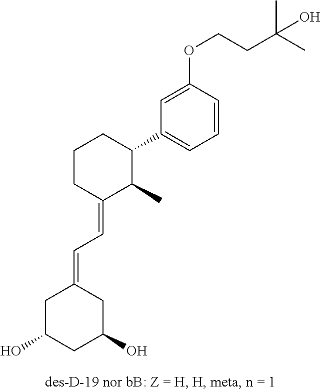 [SMILES: C[C@H]1/C(=C/C=C2C[C@@H](O)C[C@H](O)C2)CCC[C@@H]1c1cccc(OCCC(C)(C)O)c1]

Similar to the synthesis of compound des-D-19-nor aB from compound 12aB, compound des-D-19-nor bB (19 mg) was synthesized in a yield of 46% from compound 12bB (48.2 mg, 0.1 mmol) and compound 22b (53 mg, 0.11 mmol).

¹H NMR (600 MHz, CDCl₃) δ 7.21 (dd, J=7.8, 8.4 Hz, 1H, Ar), 6.79 (d, J=7.8 Hz, 1H, Ar), 6.76 (s, 1H, Ar), 6.75 (d, J=8.4 Hz, 1H, Ar), 6.34 (d, J=11.4 Hz, 1H, CH═C), 6.10 (d, J=11.4 Hz, 1H, CH═C), 4.18 (t, J=6.0 Hz, 2H, OCH₂), 4.16-4.06 (m, 2H, CHO), 2.94 (brd, J=13.2 Hz, 1H, CHAr), 2.78 (dd, J=3.6, 13.2 Hz, 1H), 2.51 (dd, J=3.0, 13.2 Hz, 1H), 2.48 (dd, J=3.0, 13.2 Hz, 1H), 2.41 (brs, 1H), 2.37-2.31 (m, 1H), 2.27 (dd, J=7.2, 13.2 Hz, 1H, CH₂), 2.25-2.20 (m, 2H), 2.16 (dd, J=7.2, 13.2 Hz, 1H), 2.00 (t, J=6.0 Hz, 2H, OCH₂CH₂), 2.01-1.80 (m, 3H), 1.74-1.51 (m, 3H), 1.43-1.37 (m, 1H), 1.32 (s, 6H, C(CH₃)₂), 0.86 (d, J=6.6 Hz, 3H, CHCH₃).

¹³C NMR (150 MHz, CDCl₃) δ 158.6, 148.1, 146.2, 132.4, 129.3, 124.0, 120.3, 114.8, 113.9, 111.5, 70.5, 67.4, 67.2, 65.1, 53.7, 44.8, 43.6, 42.2, 41.6, 37.3, 35.4, 29.7, 29.6, 29.5, 27.4, 16.2.
IR (ATR) 3359, 2965, 2925, 2881, 2854, 1607, 1583, 1476, 1444, 1367, 1285, 1267, 1232, 1156, 1081, 1044, 974, 940, 886, 813, 783, 753, 700 cm⁻¹.
HR-MS m/z=calcd for C₂₆H₃₈NaO₄ [M+Na]⁺: 437.26678, found 437.26497.

### Synthesis Example 16-5

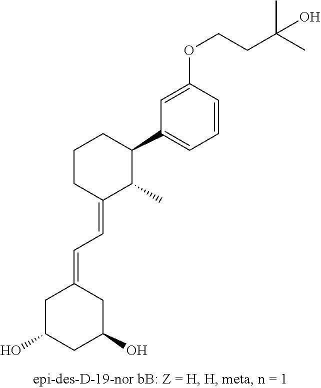 [SMILES: C[C@@H]1/C(=C/C=C2C[C@@H](O)C[C@H](O)C2)CCC[C@H]1c1cccc(OCCC(C)(C)O)c1]

Similar to the synthesis of compound des-D-19-nor aB from compound 12aB, compound epi-des-D-19-nor bB (25 mg) was synthesized in a yield of 67% from compound epi-12bB (48.0 mg, 0.1 mmol) and compound 22b (43.3 mg, 0.09 mmol).

¹H NMR (600 MHz, CDCl₃) δ 7.21 (dd, J=7.8, 8.4, 7.8 Hz, 1H, Ar), 6.79 (d, J=7.8 Hz, 1H, Ar), 6.76 (s, 1H, Ar), 6.75 (d, J=8.4 Hz, 1H, Ar), 6.35 (d, J=11.4 Hz, 1H, CH═C), 6.09 (d, J=11.4 Hz, 1H, CH═C), 4.18 (t, J=6.0 Hz, 2H, OCH₂), 4.12 (brs, 2H, CHO), 2.94 (brd, J=13.2 Hz, 1H, CHAr), 2.66 (dd, J=3.6, 13.2 Hz, 1H), 2.53 (dd, J=3.6, 13.2 Hz, 1H), 2.39 (brs, 1H, OH), 2.37 (dd, J=7.2, 13.2 Hz, 1H), 2.40-2.31 (m, 1H), 2.24-2.20 (m, 2H), 2.00 (t, J=6.0 Hz, 2H, OCH₂CH₂), 1.95-1.86 (m, 4H), 1.69 (dq, J=3.6, 13.2 Hz, 1H), 1.55 (brd, 1H), 1.48 (brd, 1H), 1.44-1.36 (tq, J=3.6, 13.2 Hz, 1H), 1.32 (s, 6H, C(CH₃)₂), 0.86 (d, J=6.6 Hz, 3H, CHCH₃).

¹³C NMR (150 MHz, CDCl₃) δ 158.6, 148.1, 146.1, 132.5, 129.3, 123.9, 120.3, 114.8, 113.9, 111.5, 70.5, 67.4, 67.1, 65.0, 53.7, 45.0, 43.6, 42.2, 41.6, 36.9, 35.4, 29.7, 29.6, 27.4, 16.1.
IR (ATR) 3366, 2966, 2926, 2877, 2854, 1607, 1583, 1476, 1443, 1367, 1314, 1285, 1267, 1244, 1156, 1081, 1043, 975, 940, 909, 878, 812, 783, 755, 700 cm⁻¹.
HR-MS m/z=calcd for C₂₆H₃₈NaO₄ [M+Na]⁺: 437.26678, found 437.26508.

### Synthesis Example 16-6

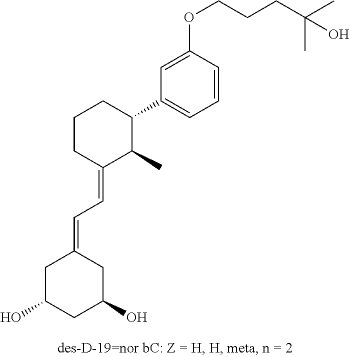 [SMILES: C[C@H]1/C(=C/C=C2C[C@@H](O)C[C@H](O)C2)CCC[C@@H]1c1cccc(OCCCC(C)(C)O)c1]

Similar to the synthesis of compound des-D-19-nor aB from compound 12aB, compound des-D-19-nor bC (21.4 mg) was synthesized in a yield of 50% from compound 12bC (49.6 mg, 0.10 mmol) and compound 22b (53.0 mg, 0.11 mmol).

¹H NMR (600 MHz, CDCl₃) δ 7.20 (dd, J=7.2, 7.8 Hz, 1H, Ar), 6.76 (d, J=7.2 Hz, 1H, Ar), 6.74 (s, 1H, Ar), 6.73 (d, J=7.8 Hz, 1H, Ar), 6.34 (d, J=11.4 Hz, 1H, CH═C), 6.09 (d, J=11.4 Hz, 1H, CH═C), 4.13 (brs, 1H, CHO), 4.08 (brs, 1H, CHO), 3.98 (t, J=6.6 Hz, 2H, OCH₂), 2.95 (brd, J=13.2 Hz, 1H, CHAr), 2.75 (dd, J=3.0, 13.2 Hz, 1H), 2.51 (dd, J=3.0, 13.2 Hz, 1H), 2.37-2.15 (m, 4H), 1.97-1.80 (m, 8H), 1.72-1.65 (m, 3H), 1.54 (brd, 1H), 1.47 (brd, J=4.8 Hz, 1H), 1.53-1.45 (m, 2H), 1.43-1.37 (m, 1H), 1.27 (s, 6H, C(CH₃)₂), 0.85 (d, J=6.0 Hz, 3H, CHCH₃).

¹³C NMR (150 MHz, CDCl₃) δ 159.0, 148.0, 146.3, 132.2, 129.3, 124.0, 119.9, 114.6, 113.9, 111.6, 70.7, 68.2, 67.4, 67.2, 53.9, 44.8, 43.7, 42.2, 40.3, 37.2, 35.5, 29.8, 29.3 (2C), 27.5, 24.4, 16.0.
IR (ATR) 3363, 2965, 2926, 2877, 2854, 1606, 1583, 1444, 1367, 1285, 1267, 1248, 1212, 1157, 1085, 1048, 974, 939, 876, 858, 811, 790, 753, 700 cm⁻¹.
HR-MS m/z=calcd for C₂₇H₄₀NaO₄ [M+Na]⁺: 451.28243, found 451.28313.

### Synthesis Example 17

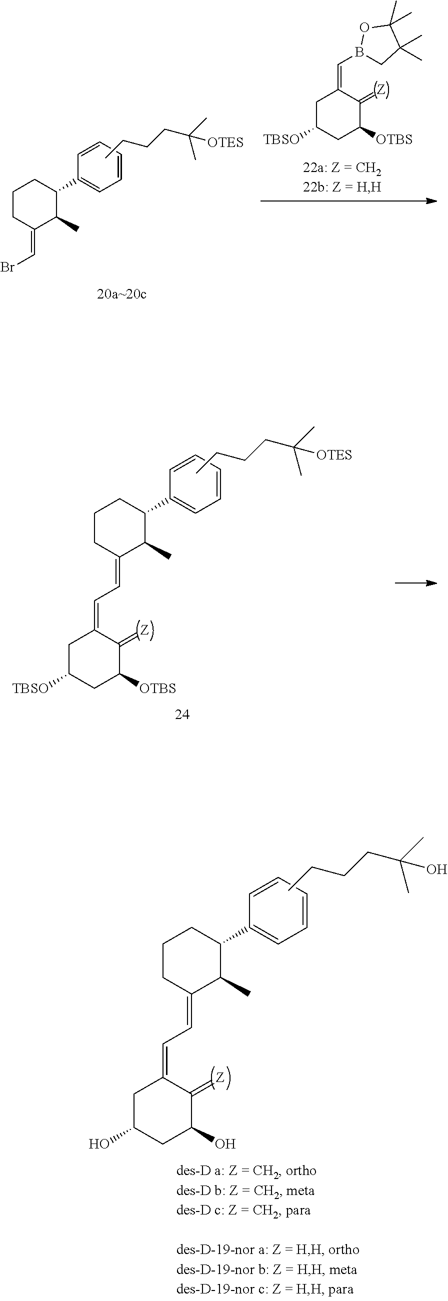

### Synthesis Example 17-1

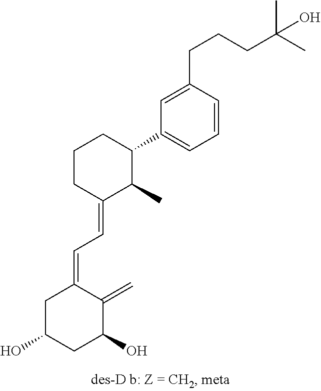 [SMILES: C=C1/C(=C\C=C2/CCC[C@H](c3cccc(CCCC(C)(C)O)c3)[C@H]2C)C[C@@H](O)C[C@@H]1O]

A solution of compound 20aB (38.3 mg, 0.11 mmol, 1.1 equivalents) and compound 22a (47.0 mg, 0.10 mmol, 1.0 equivalent) in tetrahydrofuran (1.0 mL) and a 3 N potassium hydroxide solution (56 μL) were added to 1,1′-bis(diphenylphosphino)ferrocene-palladium(II) dichloride (5.9 mg, 8.0 μmol, 8.0 mol %) and stirred at 50° C. for 12 hours. After being cooled to room temperature, the reaction solution was dried over anhydrous sodium sulfate and then filtered through Celite with diethyl ether. The resulting filtrate was concentrated under reduced pressure to give a crude product of compound 24.

A 1.0 M tetrahydrofuran solution of tetrabutylammonium fluoride (0.6 mL, 0.6 mmol, 6.0 equivalents) was added at 0° C. to a solution of the crude product of compound 24 in tetrahydrofuran (0.1 mL). After the mixture was stirred at room temperature for 12 hours, the reaction was quenched with a saturated ammonium chloride solution, followed by extraction with diethyl ether. The resulting organic layer was dried over anhydrous magnesium sulfate and then filtered, and the filtrate was concentrated. The resulting residue was purified by silica gel column chromatography (hexane/ethyl acetate/methanol) to give compound des-D b (19.1 mg) in a yield of 45%.

¹H NMR (600 MHz, CDCl₃) δ 7.20 (dd, J=7.2, 7.8 Hz, 1H, Ar), 7.01 (d, J=7.8 Hz, 1H, Ar), 6.99 (d, J=7.2 Hz, 1H, Ar), 6.98 (s, 1H, Ar), 6.42 (d, J=11.4 Hz, 1H, CH═C), 6.25 (d, J=11.4 Hz, 1H, CH═C), 5.34 (s, 1H, C═CH₂), 5.04 (s, 1H, C═CH₂), 4.44 (brt, J=4.8 Hz, 1H, CHO), 4.23 (brs, 1H, CHO), 2.98 (brd, J=13.2 Hz, 1H, CHAr), 2.67-2.61 (m, 1H), 2.60 (t, J=7.5 Hz, 2H, ArCH₂), 2.33 (dd, J=7.2, 13.2 Hz, 1H), 2.35-2.29 (m, 1H). 2.20 (dt, J=3.6, 11.4 Hz, 1H), 1.98 (t, J=5.4 Hz, 2H), 1.96-1.82 (m, 5H), 1.73-1.64 (m, 4H), 1.54-1.46 (m, 2H), 1.42 (tq, J=3.6, 13.2 Hz, 1H), 1.20 (s, 6H, C(CH₃)₂O), 0.77 (d, J=6.6 Hz, 3H, CHCH₃).

¹³C NMR (150 MHz, CDCl₃) δ 147.5, 146.4, 146.3, 142.4, 134.1, 128.2, 127.5, 126.0, 125.2, 124.7, 116.5, 112.4, 71.1, 71.0, 66.7, 53.7, 45.4, 43.7, 43.5, 42.8, 36.3, 35.6, 29.9, 29.2, 27.5, 26.3, 16.0.
IR (ATR) 3362, 2964, 2926, 2855, 1715, 1658, 1647, 1605, 1487, 1443, 1368, 1217, 1146, 1119, 1053, 957, 910, 797, 754, 707 cm⁻¹.
HR-MS m/z=calcd for C₂₈H₄₀NaO₃ [M+Na]⁺: 447.28751, found 447.28969.

### Synthesis Example 17-2

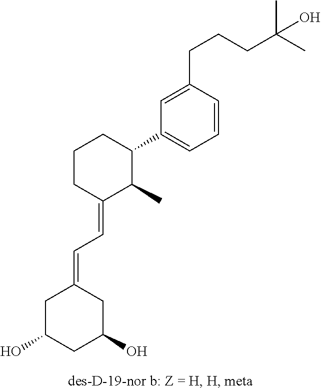 [SMILES: C[C@H]1/C(=C/C=C2C[C@@H](O)C[C@H](O)C2)CCC[C@@H]1c1cccc(CCCC(C)(C)O)c1]

Similar to the synthesis of compound des-D b from compound 20aB, compound des-D-19-nor b (57.0 mg) was synthesized in a yield of 69% from compound 20aB (76.6 mg, 0.22 mmol) and compound 22b (96.5 mg, 0.20 mmol).

¹H NMR (600 MHz, CDCl₃) δ 7.20 (dd, J=7.2, 7.8 Hz, 1H, Ar), 7.02 (d, J=7.8 Hz, 1H, Ar), 7.00 (d, J=7.2 Hz, 1H, Ar), 6.99 (s, 1H, Ar), 6.35 (d, J=11.4 Hz, 1H, CH═C), 6.09 (d, J=11.4 Hz, 1H, CH═C), 4.13 (brs, 1H, CHO), 4.08 (brs, 1H, CHO), 2.96 (brd, J=13.2 Hz, 1H, CHAr), 2.75 (dd, J=3.6, 13.2 Hz, 1H), 2.60 (t, J=7.8 Hz, 2H, ArCH₂), 2.51 (dd, J=3.6, 13.2 Hz, 1H), 2.34 (dq, J=10.8, 6.0 Hz, 1H), 2.27 (dd, J=7.2, 13.2 Hz, 1H), 2.24 (dd, J=7.2, 13.2 Hz, 1H), 2.22 (dd, J=3.6, 10.8 Hz, 1H), 1.97-1.80 (m, 5H), 1.74-1.66 (m, 3H), 1.64 (brs, 1H), 1.56 (brs, 1H), 1.53-1.48 (m, 2H), 1.40 (tq, J=3.6, 12.6 Hz, 1H), 1.21 (s, 6H, C(CH₃)₂), 0.83 (d, J=6.6 Hz, 3H, CHCH₃).

¹³C NMR (150 MHz, CDCl₃) δ 146.4, 146.2, 142.4, 132.4, 128.2, 127.5, 126.1, 124.8, 123.9, 114.6, 71.0, 67.4, 67.1, 53.8, 44.8, 43.7, 43.5, 42.1, 37.1, 36.3, 35.6, 29.8, 29.2 (C2), 27.6, 26.2, 16.0.
IR (ATR) 3358, 2965, 2927, 2877, 2854, 1606, 1486, 1443, 1366, 1302, 1215, 1149, 1122, 1084, 1047, 974, 940, 907, 892, 795, 753, 706 cm⁻¹.
HR-MS m/z=calcd for C₂₇H₄₀NaO₃ [M+Na]⁺: 435.28751, found 435.28765.

### Synthesis Example 18

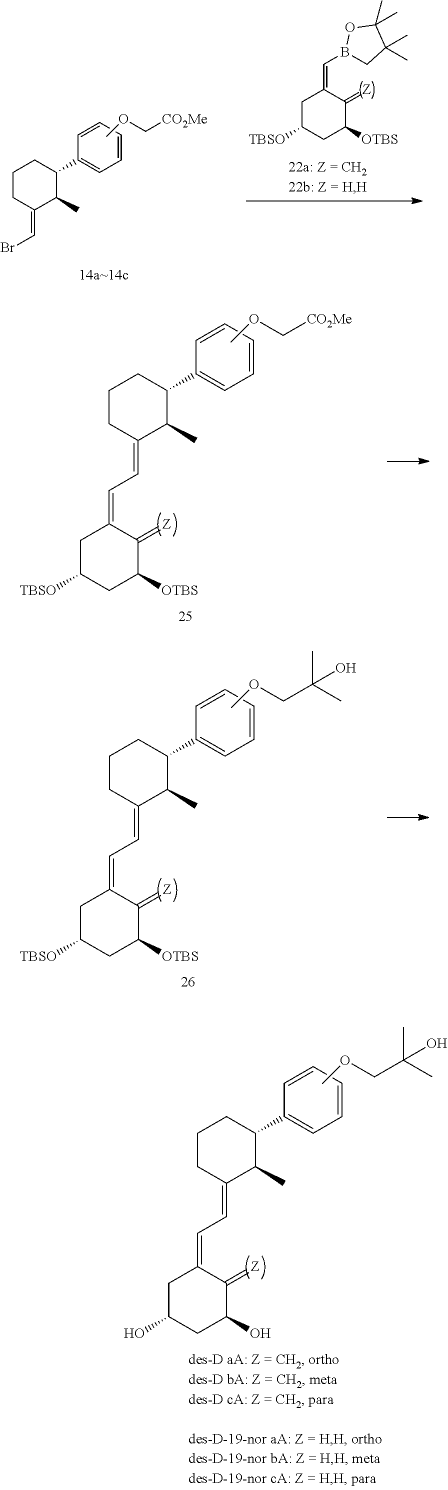

### Synthesis Example 18-1

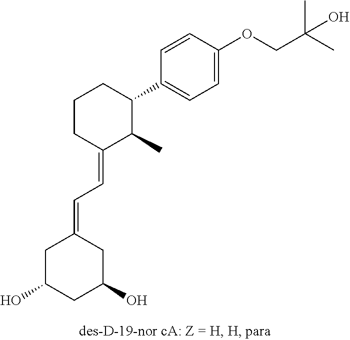 [SMILES: C[C@H]1/C(=C/C=C2C[C@@H](O)C[C@H](O)C2)CCC[C@@H]1c1ccc(OCC(C)(C)O)cc1]

A solution of compound 14c (93.8 mg, 0.26 mmol, 1.0 equivalent) and compound 22b (137 mg, 0.28 mmol, 1.1 equivalents) in tetrahydrofuran (2.8 mL) and a 3 N potassium hydroxide solution (0.15 mL) were added to 1,1′-bis(diphenylphosphino)ferrocene-palladium(II) dichloride (16.4 mg, 22.4 μmol, 8.0 mol %) and stirred at 50° C. for 12 hours. After being cooled to room temperature, the reaction solution was dried over anhydrous sodium sulfate and then filtered through silica gel with diethyl ether. The resulting filtrate was concentrated under reduced pressure to give a crude product of compound 25.

A 0.38 M diethyl ether solution of methylmagnesium iodide (1.5 mL, 0.57 mmol, 2.2 equivalents) was added at 0° C. to a solution of the crude product of compound 25 in tetrahydrofuran (2.6 mL). After the mixture was stirred at room temperature for 1 hour, a saturated ammonium chloride solution was added to the reaction mixture, followed by extraction with diethyl ether. The organic layer was washed with a saturated aqueous sodium chloride solution, then dried over anhydrous sodium sulfate, and then filtered through silica gel with diethyl ether. The resulting filtrate was concentrated under reduced pressure to give a crude product of compound 26.

Tetrabutylammonium fluoride (1.56 mL, 1.56 mmol, 6.0 equivalents) was added at 0° C. to a solution of the crude product of compound 26 in tetrahydrofuran (0.26 mL). After the mixture was stirred at room temperature for 12 hours, the reaction was quenched with a saturated ammonium chloride solution, followed by extraction with diethyl ether. The resulting organic layer was dried over anhydrous magnesium sulfate and then filtered, and the filtrate was concentrated. The resulting residue was purified by silica gel column chromatography (hexane/ethyl acetate/methanol) to give compound des-D-19-nor cA (70.7 mg) in a yield of 69%.

¹H NMR (600 MHz, CDCl₃) δ 7.09 (d, J=7.8 Hz, 2H, Ar), 6.86 (d, J=7.8 Hz, 2H, Ar), 6.35 (d, J=10.8 Hz, 1H, CH═C), 6.09 (d, J=10.8 Hz, 1H, CH═C), 4.16 (brs, 1H, CHO), 4.08 (brs, 1H, CHO), 3.78 (s, 2H, OCH₂), 2.97 (brd, J=13.2 Hz, 1H, CHAr), 2.75 (dd, J=3.6, 13.2 Hz, 1H), 2.51 (dd, J=3.6, 13.2 Hz, 1H), 2.32-2.27 (m, 2H), 2.24 (dd, J=6.6, 13.2 Hz, 1H), 2.18 (dt, J=3.6, 12.0 Hz, 1H), 1.97-1.80 (m, 5H), 1.67 (dq, J=3.6, 13.2 Hz, 1H), 1.59 (brs, 1H), 1.51 (brs, 1H), 1.48 (brs, 1H), 1.40 (brd, J=12.6 Hz, 1H), 1.34 (s, 6H, C(CH₃)₂), 0.83 (d, J=6.6 Hz, 3H, CHCH₃).

¹³C NMR (150 MHz, CDCl₃) δ 157.0, 146.3, 138.9, 132.4, 128.2, 123.9, 114.5, 114.4, 75.9, 70.1, 67.3, 67.1, 53.0, 44.8, 44.0, 42.2, 37.1, 35.8, 29.9, 27.6, 26.1, 15.9.
IR (ATR) 3358, 2968, 2925, 2875, 1609, 1510, 1456, 1366, 1301, 1235, 1176, 1046, 975, 923, 851, 825, 754 cm⁻¹.
HR-MS m/z=calcd for C₂₅H₃₆NaO₄ [M+Na]⁺: 423.25113, found 423.25293.

### Synthesis Example 19

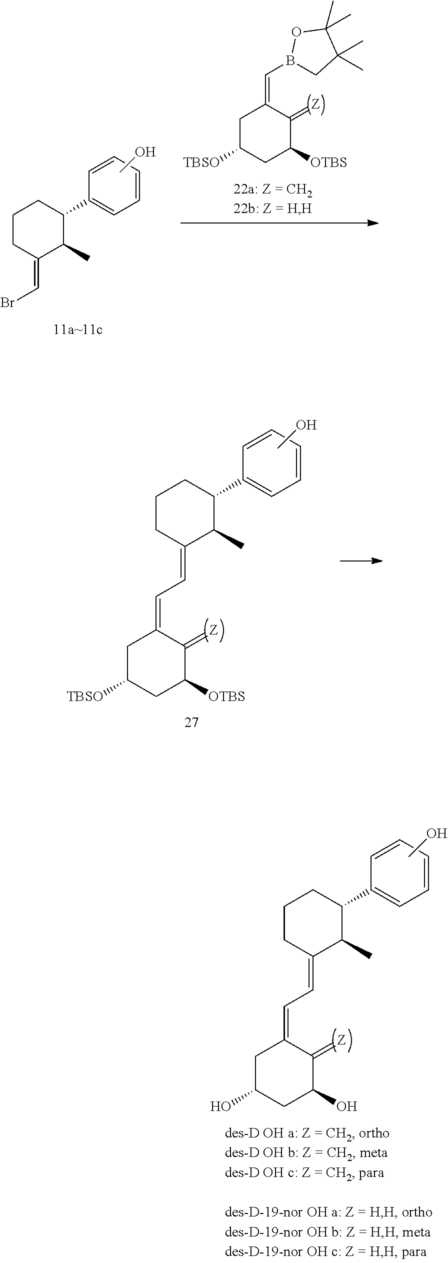

### Synthesis Example 19-1

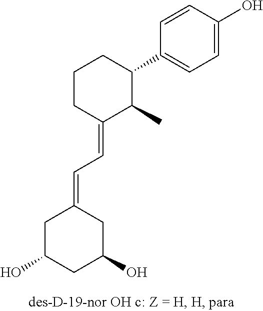 [SMILES: C[C@H]1/C(=C/C=C2C[C@@H](O)C[C@H](O)C2)CCC[C@@H]1c1ccc(O)cc1]

A solution of compound 11c (7.6 mg, 27 μmol, 1.0 equivalent) and compound 22b (14.5 mg, 30 μmol, 1.1 equivalents) in tetrahydrofuran (0.3 mL) and a 3 N potassium hydroxide solution (18 μL) were added to 1,1′-bis(diphenylphosphino)ferrocene-palladium(II) dichloride (1.6 mg, 2.2 μmol, 8.0 mol %) and stirred at 50° C. for 12 hours. After being cooled to room temperature, the reaction solution was dried over anhydrous sodium sulfate and then filtered through Celite with diethyl ether. The resulting filtrate was concentrated under reduced pressure to give a crude product of compound 27.

A 1.0 M tetrahydrofuran solution of tetrabutylammonium fluoride (70 μL, 70 μmol, 2.5 equivalents) was added at 0° C. to a solution of the crude product of compound 27 in tetrahydrofuran (0.3 mL). After the mixture was stirred at room temperature for 12 hours, the reaction was quenched with a saturated ammonium chloride solution, followed by extraction with diethyl ether. The resulting organic layer was dried over anhydrous magnesium sulfate and then filtered, and the filtrate was concentrated. The resulting residue was purified by silica gel column chromatography (hexane/ethyl acetate/methanol) to give compound des-D-19-nor OH c (5.0 mg) in a yield of 56%.

¹H NMR (600 MHz, CD₃OD) δ 7.03 (d, J=7.8 Hz, 2H, Ar), 6.73 (d, J=7.8 Hz, 2H, Ar), 6.28 (d, J=10.8 Hz, 1H, CH═C), 6.14 (d, J=10.8 Hz, 1H, CH═C), 4.11-4.03 (m, 2H, CHO), 3.01 (brd, J=12.6 Hz, 1H, CHAr), 2.64 (dd, J=3.6, 13.8 Hz, 1H), 2.47 (dd, J=3.6, 13.8 Hz, 1H), 2.30 (dd, J=7.2, 13.8 Hz, 2H), 2.34-2.27 (m, 1H), 2.22 (dd, J=7.2, 13.8 Hz, 1H), 2.13 (dt, J=3.6, 12.0 Hz, 1H), 1.97-1.79 (m, 5H), 1.72 (dq, J=3.0, 12.6 Hz, 1H), 1.40 (tq, J=3.6, 12.6 Hz, 1H), 0.85 (d, J=6.6 Hz, 3H, CHCH₃).

¹³C NMR (150 MHz, CD₃OD) δ 1574, 146.9, 135.6, 130.1, 129.8, 124.7, 117.0, 116.9, 68.9, 68.6, 55.6, 46.4, 46.1, 43.5, 38.5, 38.0, 31.7, 29.7, 17.3.
IR (ATR) 3334, 2920, 2850, 1612, 1567, 1514, 1453, 1369, 1231, 1098, 1045, 974, 827, 752 cm⁻¹.
HR-MS m/z=calcd for C₂₁H₂₈NaO₃ [M+Na]⁺: 351.19361, found 351.19372.

### Synthesis Example 20

In the formula below, “Ph” represents a phenyl group.

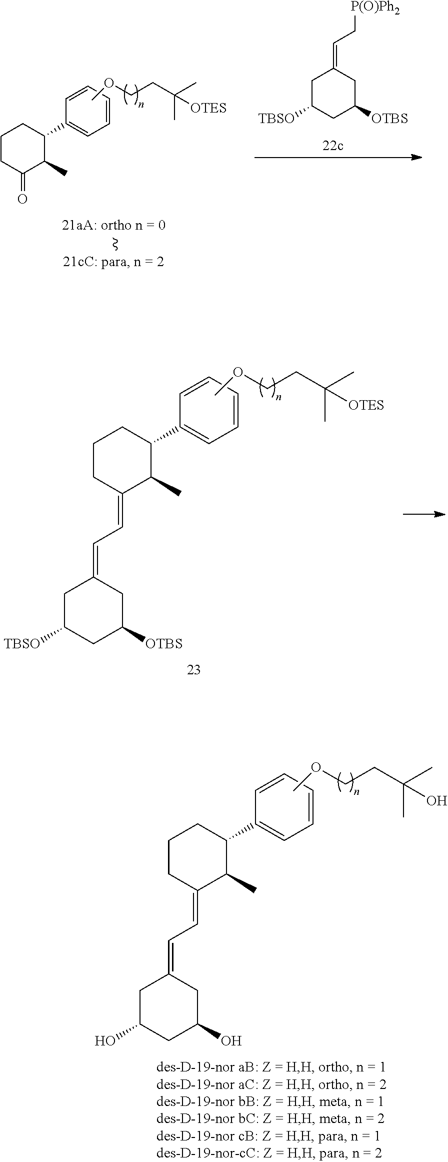

### Synthesis Example 20-1

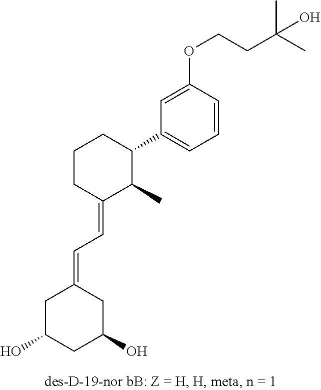 [SMILES: C[C@H]1/C(=C/C=C2C[C@@H](O)C[C@H](O)C2)CCC[C@@H]1c1cccc(OCCC(C)(C)O)c1]

A 1.59 M hexane solution of n-butyllithium (18 μL, 19.1 μmol) was added at−78° C. to a solution of compound 22c (15.8 mg, 27.7 μmol, 1.0 equivalent) in tetrahydrofuran (0.27 mL). After the mixture was stirred at −78° C. for 30 minutes, a solution of compound 21bB (13.4 mg, 33.2 μmol) in tetrahydrofuran (0.33 mL) was added at −78° C. to the mixture. After the mixture was stirred at 0° C. for 3 hours, a saturated ammonium chloride solution was added to the reaction mixture, followed by extraction with diethyl ether. The organic layer was washed with a saturated aqueous sodium chloride solution, then dried over anhydrous sodium sulfate, and then filtered through Celite with diethyl ether. The resulting filtrate was concentrated under reduced pressure to give a crude product of compound 23.

A 1.0 M tetrahydrofuran solution of tetrabutylammonium fluoride (0.17 mL, 166 μmol, 6.0 equivalents) was added at 0° C. to a solution of the crude product of compound 23 in tetrahydrofuran (0.28 mL). After the mixture was stirred at room temperature for 18 hours, the reaction was quenched with a saturated ammonium chloride solution, followed by extraction with diethyl ether. The resulting organic layer was dried over anhydrous magnesium sulfate and then filtered, and the filtrate was concentrated. The resulting residue was purified by silica gel column chromatography (hexane/ethyl acetate/methanol) to give compound des-D-19-nor bB (4.8 mg) in a yield of 42%.

The resulting ¹H NMR, ¹³C NMR, IR, and HR-MS data are the same as those shown in Synthesis Example 16-4.

### Synthesis Example 21

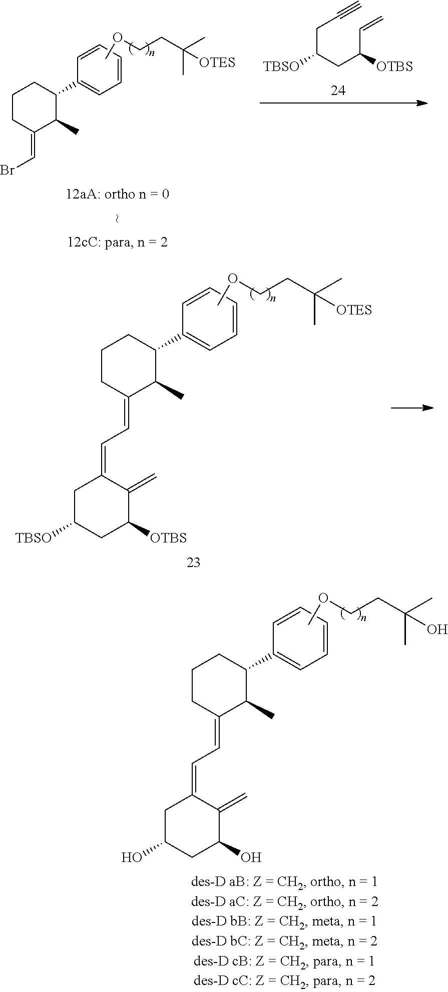

### Synthesis Example 21-1

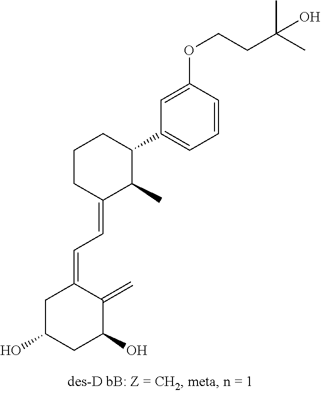 [SMILES: C=C1/C(=C\C=C2/CCC[C@H](c3cccc(OCCC(C)(C)O)c3)[C@H]2C)C[C@@H](O)C[C@@H]1O]

A solution of compound 24 (25 mg, 67.8 μmol, 1.0 equivalent) and compound 12bB (65.3 mg, 135.6 μmol, 2.0 equivalents) in triethylamine (0.5 mL) was added to a solution of palladium(II) acetate (3.0 mg, 13.6 μmol, 20 mol %) and triphenylphosphine (14.3 mg, 54.4 μmol, 80 mol %) in toluene (0.17 mL) and stirred at 80° C. for 2 hours. After being cooled to room temperature, the reaction mixture was filtered through Celite with hexane. The resulting filtrate was concentrated under reduced pressure to give a crude product of compound 23.

A 1.0 M tetrahydrofuran solution of tetrabutylammonium fluoride (0.5 mL, 0.5 mmol, 7.4 equivalents) was added at 0° C. to a solution of the crude product of compound 23 in tetrahydrofuran (0.1 mL). After the mixture was stirred at room temperature for 12 hours, the reaction was quenched with a saturated ammonium chloride solution, followed by extraction with diethyl ether. The resulting organic layer was dried over anhydrous magnesium sulfate and then filtered, and the filtrate was concentrated. The resulting residue was purified by silica gel column chromatography (hexane/ethyl acetate/methanol) to give compound des-D-19-nor bB (9.4 mg) in a yield of 34%.

The resulting ¹H NMR, ¹³C NMR, IR, and HR-MS data are the same as those shown in Synthesis Example 16-3.

### Synthesis Example 22

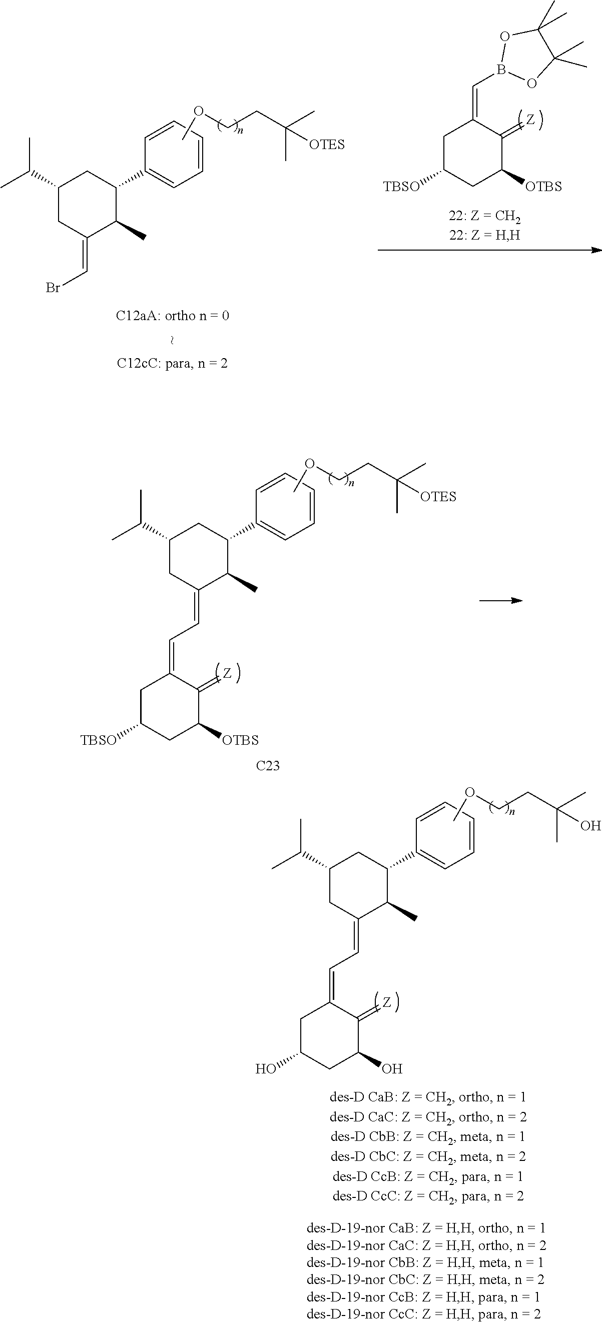

### Synthesis Example 22-1

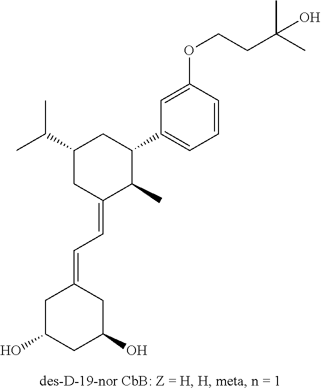 [SMILES: CC(C)[C@H]1C/C(=C\C=C2C[C@@H](O)C[C@H](O)C2)[C@H](C)[C@@H](c2cccc(OCCC(C)(C)O)c2)C1]

A solution of compound C12bB (57.6 mg, 0.11 mmol, 1.1 equivalents) and compound 22b (48.3 mg, 0.1 mmol, 1.0 equivalent) in tetrahydrofuran (1.0 mL) and a 3 N potassium hydroxide solution (56 μL) were added to 1,1′-bis(diphenylphosphino)ferrocene-palladium(II) dichloride (5.8 mg, 8 μmol, 8.0 mol %) and stirred at 50° C. for 12 hours. After being cooled to room temperature, the reaction solution was dried over anhydrous sodium sulfate and then filtered through Celite with diethyl ether. The resulting filtrate was concentrated under reduced pressure to give a crude product of compound C23.

A 1.0 M tetrahydrofuran solution of tetrabutylammonium fluoride (0.6 mL, 0.6 mmol, 6.0 equivalents) was added at 0° C. to a solution of the crude product of compound C23 in tetrahydrofuran (0.1 mL). After the mixture was stirred at room temperature for 12 hours, the reaction was quenched with a saturated ammonium chloride solution, followed by extraction with diethyl ether. The resulting organic layer was dried over anhydrous magnesium sulfate and then filtered, and the filtrate was concentrated. The resulting residue was purified by silica gel column chromatography (hexane/ethyl acetate/methanol) to give compound des-D-19-nor CbB (35.3 mg) in a yield of 77%.

¹H NMR (600 MHz, CDCl₃) δ 7.22 (dd, J=7.8, 8.4 Hz, 1H, Ar), 6.79 (d, J=7.8 Hz, 1H, Ar), 6.76 (dd, J=1.8, 8.4 Hz, 1H, Ar), 6.74 (d, J=1.8 Hz, 1H, Ar), 6.34 (d, J=10.8 Hz, 1H, CH═C), 6.08 (d, J=10.8 Hz, 1H, CH═C), 4.19 (t, J=6.0 Hz, 2H, OCH₂), 4.14 (brs, 1H, CHO), 4.08 (brs, 1H, CHO), 2.96 (brd, J=13.2 Hz, 1H, CHAr), 2.75 (dd, J=3.6, 13.2 Hz, 1H), 2.52 (dd, J=3.6, 13.2 Hz, 1H), 2.37 (brs, 1H), 2.29-2.23 (m, 3H), 2.17 (dt, J=3.0, 12.0 Hz, 1H), 2.00 (t, J=6.0 Hz, 2H, OCH₂CH₂), 1.98-1.93 (m, 1H), 1.86 (dq, J=13.2, 1.8 Hz, 1H), 1.82 (ddd, J=3.6, 8.4, 13.2 Hz, 1H), 1.61 (t, J=12.6 Hz, 1H), 1.58-1.51 (m, 3H), 1.41 (q, J=12.6 Hz, 1H), 1.32 (s, 6H, C(CH₃)₂), 1.31-1.23 (m, 1H), 0.93 (d, J=6.6 Hz, 3H, CH(CH₃)₂), 0.89 (d, J=6.6 Hz, 3H, CH(CH₃)₂), 0.84 (d, J=6.0 Hz, 3H, CHCH₃).

¹³C NMR (150 MHz, CDCl₃) δ 158.6, 148.2, 146.0, 132.5, 129.4, 123.8, 120.2, 114.6, 113.8, 111.6, 70.4, 67.4, 67.1, 65.0, 53.6, 45.2, 44.8, 43.5, 42.2, 41.7, 38.9, 37.2, 33.5, 32.9, 29.6, 19.9, 19.4, 15.6.
IR (ATR) 3356, 2961, 2930, 2873, 1607, 1583, 1476, 1448, 1384, 1367, 1309, 1288, 1262, 1216, 1156, 1085, 1043, 973, 940, 872, 812, 783, 754, 729 cm⁻¹.
HR-MS m/z=calcd for C₂₉H₄₄NaO₄ [M+Na]⁺: 479.31373, found 479.31490.

### Synthesis Example 22-2

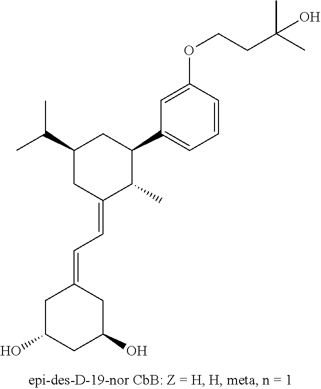 [SMILES: CC(C)[C@@H]1C/C(=C\C=C2C[C@@H](O)C[C@H](O)C2)[C@@H](C)[C@H](c2cccc(OCCC(C)(C)O)c2)C1]

Similar to the synthesis of compound des-D-19-nor CbB from compound C12bB, compound epi-des-D-19-nor CbB (33.4 mg) was synthesized in a yield of 73% from compound epi-C12bB (57.6 mg, 0.11 mmol) and compound 22b (48.3 mg, 0.1 mmol).

¹H NMR (600 MHz, CDCl₃) δ 7.22 (dd, J=7.8, 8.4 Hz, 1H, Ar), 6.79 (d, J=7.8 Hz, 1H, Ar), 6.76 (dd, J=8.4, 1.8 Hz, 1H, Ar), 6.74 (d, J=1.8 Hz, 1H, Ar), 6.36 (d, J=10.8 Hz, 1H, CH═C), 6.08 (d, J=10.8 Hz, 1H, CH═C), 4.19 (t, J=6.0 Hz, 2H, OCH₂), 4.12 (brs, 2H, CHO), 2.95 (brd, J=13.2 Hz, 1H, CHAr), 2.67 (dd, J=3.6, 13.2 Hz, 1H), 2.54 (dd, J=3.6, 13.2 Hz), 2.37-2.34 (m, 2H), 2.29-2.22 (m, 2H), 2.16 (dt, J=3.0, 12.0 Hz, 1H), 2.00 (t, J=6.0 Hz, 2H, OCH₂CH₂), 1.93-1.85 (m, 3H), 1.63-1.47 (m, 5H), 1.40 (q, J=12.6 Hz, 1H), 1.32 (s, 6H, C(CH₃)₂), 1.32-1.24 (m, 1H), 0.93 (d, J=6.6 Hz, 3H, CH(CH₃)₂), 0.90 (d, J=6.6 Hz, 3H, CH(CH₃)₂), 0.84 (d, J=6.6 Hz, 3H, CHCH₃).

¹³C NMR (150 MHz, CDCl₃) δ 158.6, 148.1, 146.0, 132.5, 129.3, 123.8, 120.2, 114.6, 113.9, 111.5, 70.4, 67.4, 67.1, 65.0, 53.6, 45.1, 45.0, 43.5, 42.1, 41.7, 38.8, 36.9, 33.5, 32.8, 29.6, 20.0, 19.3, 15.6.
IR (ATR) 3358, 2960, 2930, 2873, 1606, 1583, 1476, 1448, 1385, 1367, 1313, 1288, 1262, 1217, 1156, 1081, 1042, 974, 939, 907, 891, 872, 810, 783, 755, 728, 701 cm⁻¹.
HR-MS m/z=calcd for C₂₉H₄₄NaO₄ [M+Na]⁺: 479.31373, found 479.31486.

### Synthesis Example 23

In the formula below, “nPr” represents a n-propyl group.

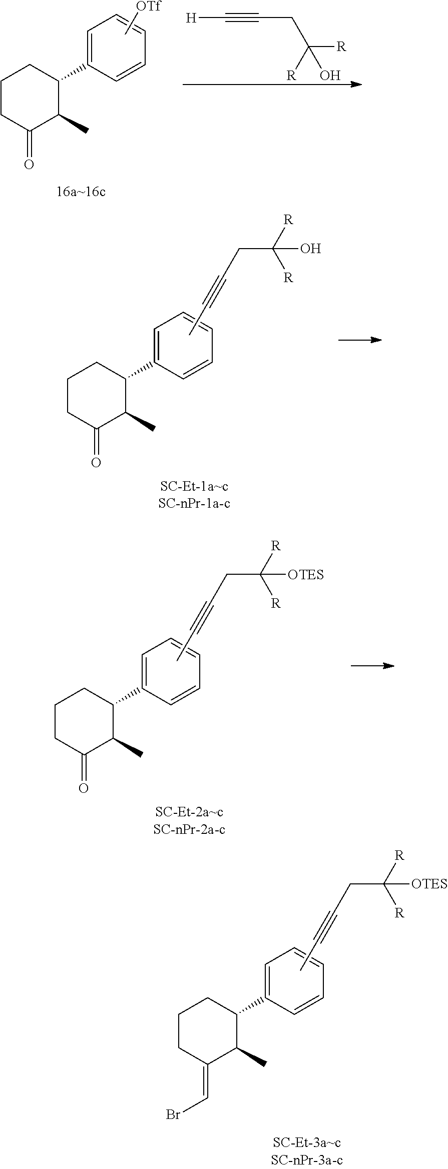

### Synthesis Example 23-1

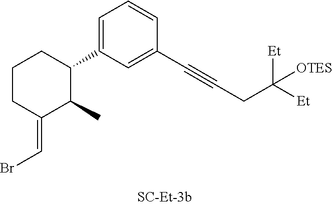

Similar to the synthesis of compound 17b from compound 16b, a crude product of compound SC-Et-1b was synthesized from compound 16b (152 mg, 0.45 mmol) and 3-ethylhex-5-yn-3-ol (85 mg, 0.675 mmol).

Triethylsilyl chloride (0.23 mL, 1.35 mmol) was added at room temperature to a solution of the crude product of compound SC-Et-1b and imidazole (153 mg, 2.25 mmol) in N,N-dimethylformamide (2.2 mL), and the mixture was stirred for 5 hours. After a saturated aqueous sodium bicarbonate solution was added to the reaction mixture, the resulting mixture was extracted with hexane. The organic layer was washed with a saturated aqueous sodium chloride solution and then dried over anhydrous magnesium sulfate. After the organic layer was filtered, the filtrate was concentrated under reduced pressure. The resulting residue was filtered through silica gel to give a crude product of compound SC-Et-2b.

A 0.5 M tetrahydrofuran solution of lithium diisopropylamide (2.6 mL, 1.3 mmol) was added at −40° C. to a mixture of (bromomethyl)triphenylphosphonium bromide (0.59 g, 1.35 mmol) and tetrahydrofuran (2.7 mL). The mixture was stirred for 1 hour while being heated at 0° C. After the mixture was cooled again to −78° C., a solution of the crude product of compound SC-Et-2b in tetrahydrofuran (2.3 mL) was added to the mixture. The resulting mixture was stirred for 12 hours while being heated at 0° C. The reaction mixture was filtered through silica gel with hexane. The resulting filtrate was concentrated under reduced pressure. The concentrate was purified by silica gel column chromatography (hexane/ethyl acetate) to give compound SC-Et-3b (80.1 mg) in a yield of 35%.

¹H NMR (400 MHz, CDCl₃) δ 7.17-7.24 (m, 3H, Ar), 7.00-7.07 (m, 1H, Ar), 5.93 (s, 1H, C═CH), 3.07-3.14 (m, 1H, ArCH), 2.55 (s, propargylic CH₂), 2.26-2.27 (m, 1H, CH₃CH), 2.19 (dt, J=4.0, 11.6 Hz, 1H), 1.84-1.95 (m, 1H), 1.66 (q, J=6.4 Hz, 4H, CH₃CH₂C), 1.58-1.70 (m, 1H), 1.35-1.45 (m, 1H), 0.96 (t, J=8.4 Hz, 9H, CH₃CH₂Si), 0.93 (t, J=7.2 Hz, 6H, CH₃CH₂C), 0.81 (d, J=6.8 Hz, 3H, CH₃CH), 0.63 (q, J=8.4 Hz, 6H, SiCH₂).

¹³C NMR (100 MHz, CDCl₃) δ 148.3, 145.2, 130.4, 129.5, 128.4, 126.7, 124.2, 99.1, 87.6, 82.4, 77.9, 53.1, 43.6, 35.3, 32.5, 32.0, 30.6, 26.6, 15.7, 8.3, 7.2, 6.9.
TLC analysis: R_{f}=0.9 (hexane:ethyl acetate=10:1 (v/v))

### Synthesis Example 23-2

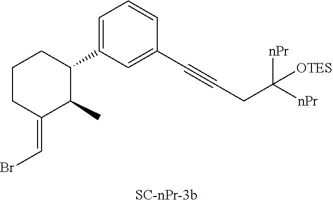

Similar to the synthesis of compound SC-Et-3b from compound 16b, compound SC-nPr-3b (110 mg) was synthesized in a yield of 49% from compound 16b (142 mg, 0.42 mmol) and 4-propylhept-1-yn-4-ol (0.11 mL, 0.63 mmol).

¹H NMR (400 MHz, CDCl₃) δ 7.18-7.26 (m, 3H, Ar), 7.06-7.09 (m, 1H, Ar), 5.94 (s, 1H, C═CH), 3.08-3.14 (m, 1H, ArCH), 2.55 (s, 2H, propargylic CH₂), 2.30-2.37 (m, 1H, CH₃CH), 2.20 (dt, J=3.2, 11.6 Hz, 1H), 1.85-1.96 (m, 3H), 1.51-1.71 (m, 5H), 1.30-1.48 (m, 4H), 0.96 (t, J=8.4 Hz, 9H, CH₃CH₂Si), 0.93 (t, J=7.6 Hz, 6H, CH₃CH₂C), 0.82 (d, J=6.4 Hz, 3H, CH₃CH), 0.62 (q, J=8.0 Hz, 6H, SiCH₂).

¹³C NMR (100 MHz, CDCl₃) δ 148.2, 145.3, 130.4, 129.5, 128.4, 126.7, 124.2, 99.1, 87.7, 82.4, 77.5, 53.1, 43.6, 42.5, 35.3, 32.4, 31.5, 26.6, 17.1, 15.7, 14.7, 7.2, 6.9.
TLC analysis: R_{f}=0.9 (hexane:ethyl acetate=10:1 (v/v))

### Synthesis Example 24

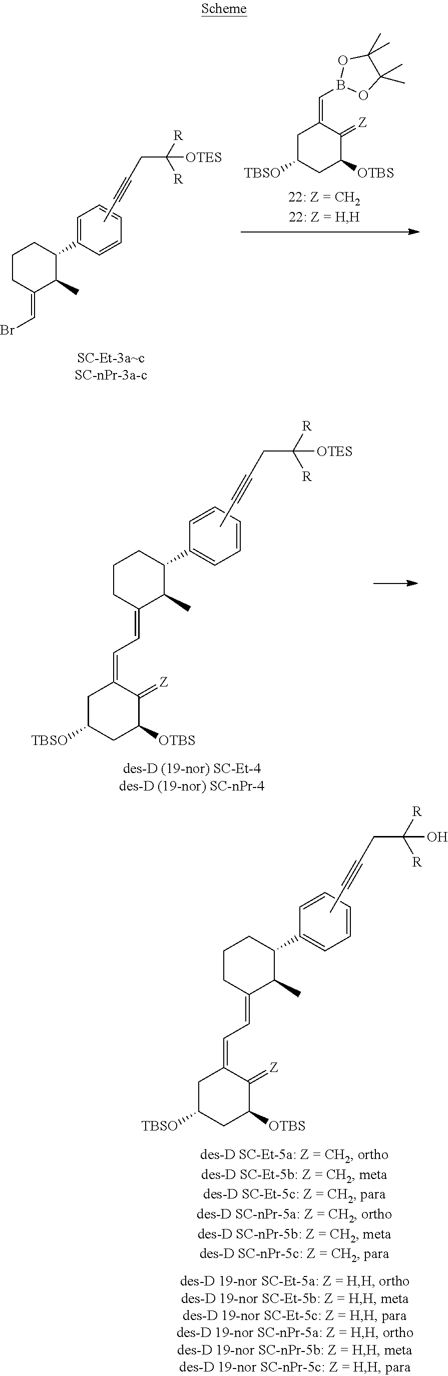

### Synthesis Example 24-1

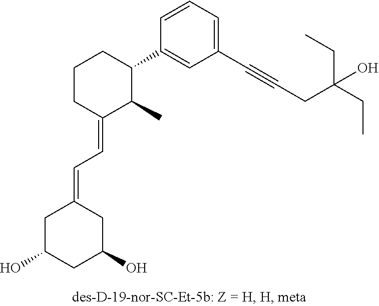 [SMILES: CCC(O)(CC)CC#Cc1cccc([C@H]2CCC/C(=C\C=C3C[C@@H](O)C[C@H](O)C3)[C@@H]2C)c1]

Similar to the synthesis of compound des-D-19-nor aB from compound 12aB and compound 22b, compound des-D-19-nor-SC-Et-5b (29.6 mg) was synthesized in a yield of 72% from compound SC-Et-3b (47.4 mg, 0.094 mmol) and compound 22b (50 mg, 0.104 mmol).

¹H NMR (400 MHz, CDCl₃) δ 7.19-7.24 (m, 3H, Ar), 7.11 (d, J=7.2 Hz, 1H, Ar), 6.34 (d, J=10.8 Hz, 1H, CH═C), 6.09 (d, J=10.8 Hz, 1H, C═CH), 4.03-4.18 (m, 2H, CHO), 2.96 (br d, J=14.0 Hz, 1H), 2.75 (br d, J=9.2 Hz, 1H), 2.58 (s, 1H, propargylic CH₂), 2.51 (br d, J=10.0 Hz, 1H), 2.11-2.39 (m, 5H), 1.58-2.00 (m, 18H), 1.22-1.43 (m, 2H), 0.94 (t, J=7.2 Hz, 6H, CH₃CH₂), 0.84 (d, J=6.4 Hz, 3H, CH₃CH).

¹³C NMR (100 MHz, CDCl₃) δ 146.0, 132.5, 130.5, 129.4, 128.3, 127.2, 123.9, 123.4, 114.7, 99.9, 85.8, 83.6, 74.2, 67.4, 67.1, 53.6, 44.8, 43.5, 42.2, 37.1, 35.5, 30.8, 30.3, 29.8, 27.5, 16.0, 8.0, 7.7.

### Synthesis Example 24-2

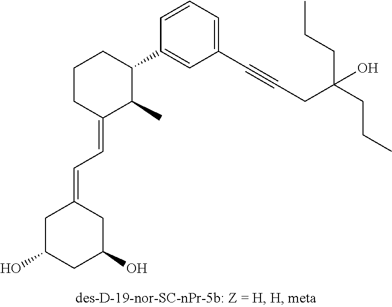 [SMILES: CCCC(O)(CC#Cc1cccc([C@H]2CCC/C(=C\C=C3C[C@@H](O)C[C@H](O)C3)[C@@H]2C)c1)CCC]

Similar to the synthesis of compound des-D-19-nor aB from compound 12aB and compound 22b, compound des-D-19 nor-SC-nPr-5b (72.8 mg) was synthesized in a yield of 90% from compound SC-nPr-3b (92.6 mg, 0.174 mmol) and compound 22b (92.4 mg, 0.191 mmol).

¹H NMR (400 MHz, CDCl₃) δ 7.18-7.26 (m, 3H, Ar), 7.11 (d, J=7.6 Hz, 1H, Ar), 6.34 (d, J=10.8 Hz, 1H, CH═C), 6.09 (d, J=10.8 Hz, 1H. C═CH), 4.03-4.20 (m, 2H, CHO), 2.96 (br d, J=14.4 Hz, 1H), 2.74 (br d, J=9.2 Hz, 1H), 2.58 (s, 2H, propargylic CH₂), 2.51 (br d, J=10.0 Hz, 1H), 2.15-2.38 (m, 3H), 1.45-2.00 (m, 9H), 1.22-1.48 (m, 7H), 0.96 (t, J=7.6 Hz, 6H, CH₃CH₂), 0.84 (d, J=6.4 Hz, 3H, CH₃CH).

¹³C NMR (100 MHz, CDCl₃) δ 146.4, 132.6, 130.5, 129.4, 128.3, 128.2, 127.2, 123.9, 123.4, 114.7, 85.8, 83.6, 73.9, 67.4, 67.1, 53.6, 44.7, 43.5, 42.1, 41.3, 37.1, 35.5, 31.3, 29.8, 27.5, 16.9, 16.0, 14.7.

### Synthesis Example 25

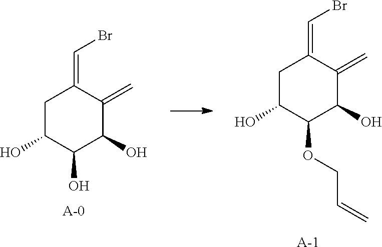

A mixture of compound A-0 (0.21 g, 0.88 mmol), Bu₂SnO (0.11 g, 0.44 mmol), n-tetrabutylammonium bromide (0.14 g, 0.44 mmol), isopropyldiethylamine (0.38 mL, 2.2 mmol), allyl bromide (0.082 mL, 0.97 mmol), and acetonitrile (4.4 mL) was stirred at 60° C. for 14 hours. After water was added to the reaction mixture, the resulting mixture was extracted with ethyl acetate. The organic layer was dried over anhydrous magnesium sulfate. After the organic layer was filtered, the filtrate was concentrated under reduced pressure. The resulting residue was purified by silica gel column chromatography to give compound A-1 (16.3 mg) in a yield of 7%.

¹H NMR (600 MHz, CDCl₃) δ 6.29 (s, 1H, BrCH═C), 5.98 (dt, J=17.4, 6.0 Hz, 1H, CH═CH₂), 5.51 (s, 1H, C═CH₂), 5.40 (s, 1H, C═CH₂), 5.33 (dd, J=1.8, 17.4 Hz, 1H, CH═CH₂), 5.24 (d, J=10.2 Hz, 1H, CH═CH₂), 4.40 (br s, 1H, CHO), 4.25 (d, J=6.6 Hz, 2H, OCH₂), 4.09 (br s, 1H, CHO), 3.59 (br s, 1H, CHO), 2.83 (d, J=7.8 Hz, 1H, OH), 2.68 (d, J=6.6 Hz, 1H, OH), 2.66 (dd, J=5.4, 13.8 Hz, 1H, ring CH₂), 2.38 (br d, J=13.8 Hz, 1H, ring CH₂).

¹³C NMR (100 MHz, CDCl₃) δ 142.4, 136.7, 134.5, 117.9, 117.4, 104.1, 78.9, 74.5, 71.0, 69.3, 40.4.

### Synthesis Example 26

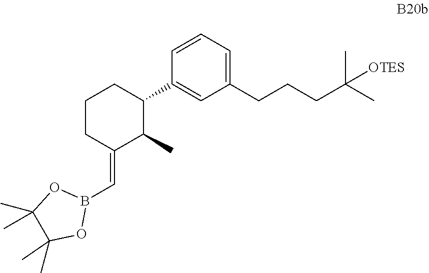

A 1.56 M hexane solution of tert-BuLi (1.40 mL, 2.18 mmol) was added dropwise at −80° C. to a solution of compound 20b (347 mg, 0.72 mmol) in diethyl ether (7.2 mL) and stirred for 1 hour. To the mixture was added 2-isopropyl-4,4,5,5-trimethyl-1,3,2-dioxaborolane (0.29 mL, 1.44 mmol), and the resulting mixture was heated to room temperature over 2 hours. The reaction was quenched by adding a saturated aqueous ammonium chloride solution, followed by extraction with diethyl ether. The organic layer was washed with a saturated aqueous sodium chloride solution and then dried over anhydrous magnesium sulfate. After the organic layer was filtered, the filtrate was concentrated under reduced pressure. The resulting residue was purified by silica gel column chromatography to give compound B20b (97.1 mg) in a yield of 26%.

¹H NMR (400 MHz, CDCl₃) δ 7.19 (t, J=7.6 Hz, 1H, Ar), 6.95-7.03 (m, 3H, Ar), 5.14 (s, 1H, C═CHB), 3.35 (br d, J=14.4 Hz, 1H, ArCH), 2.57 (t, J=7.2 Hz, 2H, ArCH₂), 2.83 (dd, J=4.8, 6.0 Hz, 1H), 2.23 (dd, J=3.6, 11.2 Hz, 1H), 1.60-2.02 (m, 8H), 1.42-1.47 (m, 3H), 1.29 (s, 12H, CH₃), 1.17 (s, 6H, CH₃), 0.93 (t. J=7.6 Hz, 9H, CH₃CH₂Si), 0.77 (d, J=6.4 Hz, 3H, CH₃CH), 0.54 (q, J=7.6 Hz, SiCH₂).

¹³C NMR (100 MHz, CDCl₃) δ 169.5, 146.1, 142.8, 128.2, 127.5, 126.1, 124.6, 82.7, 73.3, 54.1, 45.3, 44.6, 36.4, 35.8, 34.5, 29.9, 28.6, 26.3, 24.9, 24.8, 16.0, 6.8.

### Synthesis Example 27

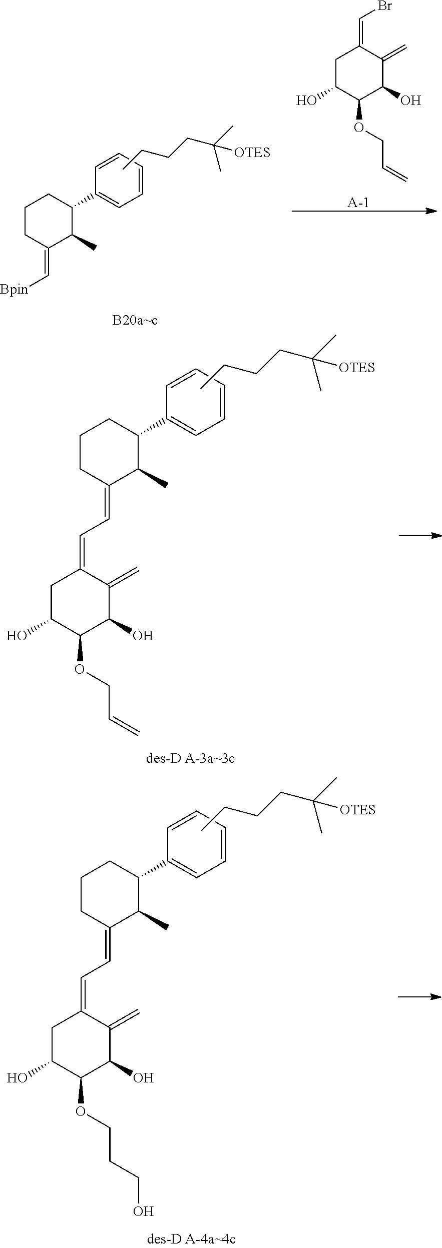

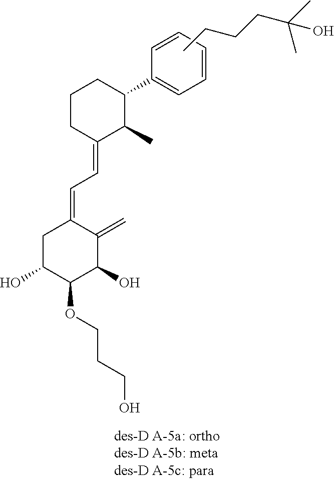

### Synthesis Example 27-1

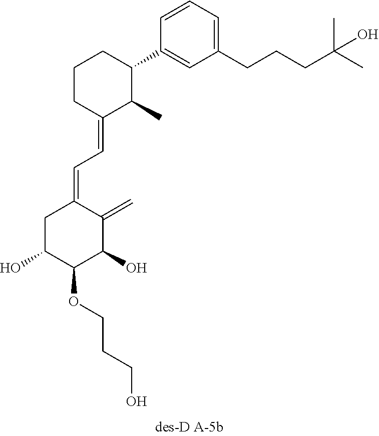 [SMILES: C=C1/C(=C\C=C2/CCC[C@H](c3cccc(CCCC(C)(C)O)c3)[C@H]2C)C[C@@H](O)[C@H](OCCCO)[C@@H]1O]

A solution of compound A-1 (8.7 mg, 0.032 mmol, 1.0 equivalent) and compound B20b (20.2 mg, 0.038 mmol, 1.19 equivalents) in tetrahydrofuran (1.0 mL) and a 3 N potassium hydroxide solution (10 μL) were added to 1,1′-bis(diphenylphosphino)ferrocene-palladium(II) dichloride (2.3 mg, 3.2 μmol, 10 mol %) and stirred at 40° C. for 6 hours. After being cooled to room temperature, the reaction solution was dried over anhydrous sodium sulfate and then filtered through Celite with diethyl ether. The resulting filtrate was concentrated under reduced pressure to give a crude product of compound des-D A-3b.

To a solution of the crude product of compound des-D A-3b in tetrahydrofuran (0.1 mL) was added a 0.5 M THF solution of 9-BBB (0.224 mL, 0.112 mmol) at 0° C. The mixture was stirred at 0° C. for 2 hours and then at room temperature for 5 hours. To the resulting mixture was added at 0° C. a 1 M aqueous NaOH solution (0.8 mL) and a 30% hydrogen peroxide solution (0.21 mL) and stirred for 30 minutes. After a saturated aqueous sodium thiosulfate solution (0.5 mL) was added to the reaction mixture, the resulting mixture was extracted with diethyl ether. The organic layer was washed with a saturated aqueous sodium chloride solution and then dried over anhydrous magnesium sulfate. After the organic layer was filtered, the filtrate was concentrated to give a crude product of compound des-D A-4b.

To a solution of the crude product of compound des-D A-4b in tetrahydrofuran (0.1 mL) was added at room temperature a 1.0 M THF solution of n-tetrabutylammonium fluoride (0.32 mL, 0.32 mmol) and stirred for 5 hours. After a saturated aqueous ammonium chloride solution was added to the reaction mixture, the resulting mixture was extracted with diethyl ether. The organic layer was dried over anhydrous magnesium sulfate. After the organic layer was filtered, the filtrate was concentrated. The resulting residue was purified by silica gel column chromatography to give compound des-D A-5b (14.1 mg) in a yield of 22%.

¹H NMR (400 MHz, CDCl₃) δ 7.15-7.20 (m, 1H, Ar), 6.93-7.03 (m, 3H, Ar), 6.51 (d, J=10.8 Hz, 1H, CH═C), 6.28 (d, J=10.8 Hz, 1H, CH═C), 5.39 (s, 1H, C═CH₂), 5.12 (s, 1H, C═CH₂), 3.75-3.90 (m, 7H, OCH₂ and CHO), 3.00 (br d, J=12 Hz, 1H, ArCH), 2.61-2.76 (m, 3H), 2.57 (t, J=7.8 Hz, 2H, ArCH₂), 2.20-2.46 (m, 3H), 1.43-1.95 (m, 9H), 1.18 (s, 6H, CH₃C), 0.78 (d, J=6.0 Hz, 3H, CH₃CH).

### Synthesis Example 28

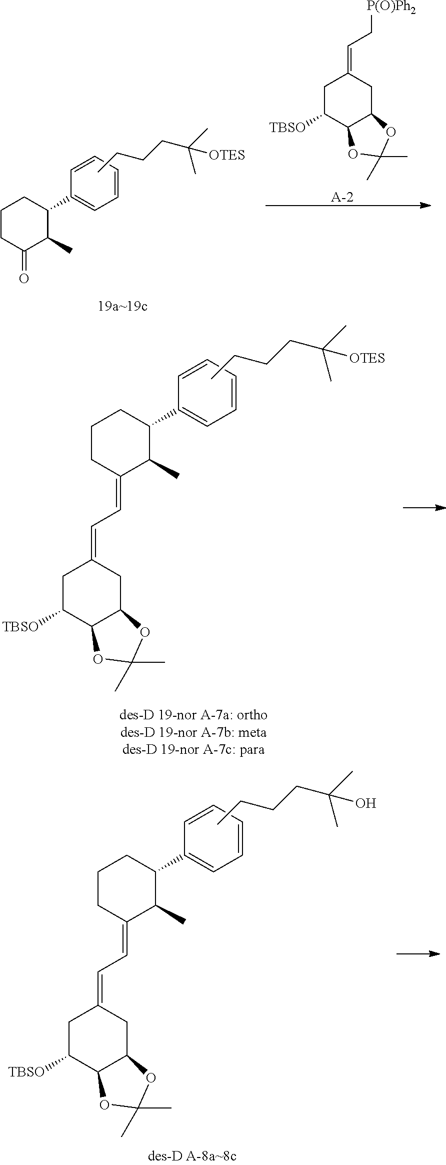

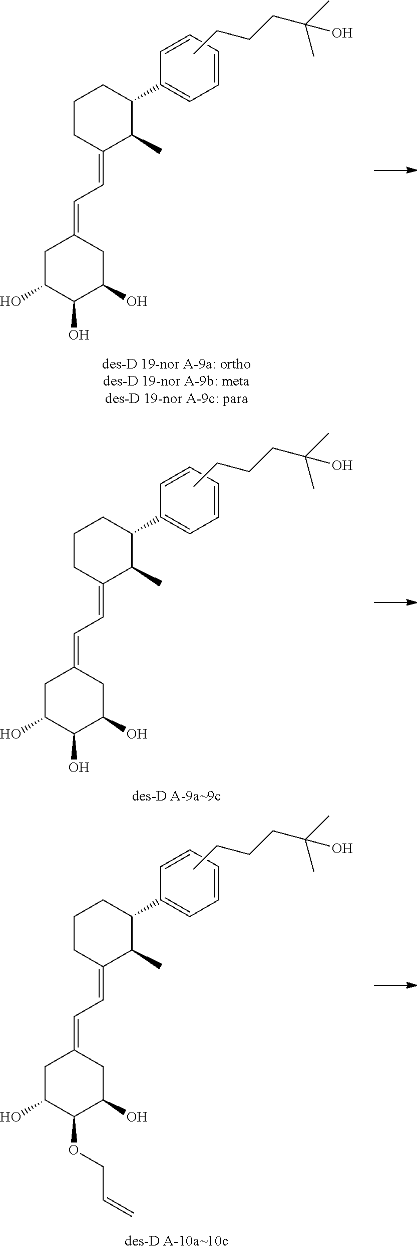

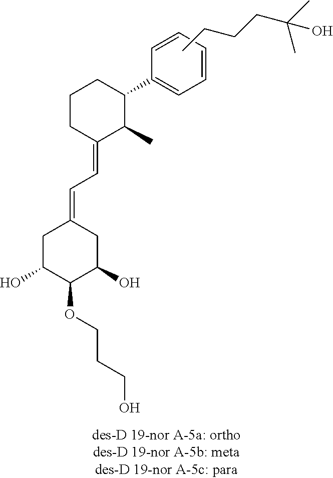

### Synthesis Example 28-1

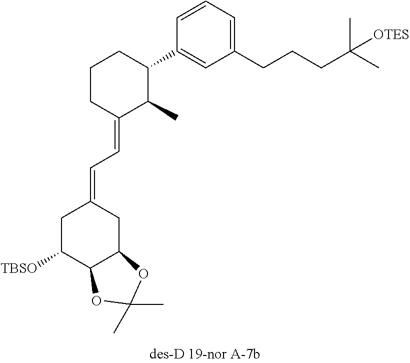

Similar to the synthesis of compound 23 from compound 22c and compound 21bB, compound des-D 19- nor A-7b (248 mg) was synthesized in a yield of 38% from compound A-2 (483 mg, 0.94 mmol) and compound 19b (480 mg, 1.2 mmol).

¹H NMR (400 MHz, CDCl₃) δ 7.20 (t, J=8.0 Hz, 1H, Ar), 6.95-7.03 (m, 3H, Ar), 6.27 (d, J=10.8 Hz, 1H, C═CH), 6.02 (d, J=10.8 Hz, 1H, C═CH), 4.18-4.23 (m, 2H, CHO), 3.89 (br d, J=9.6 Hz, 1H, CHO), 2.91-2.99 (m, 1H, ArCH), 2.62-2.72 (m, 2H), 2.57 (t, J=8.0 Hz, 2H, ArCH₂), 2.13-2.46 (m, 4H), 1.83-1.93 (m, 3H), 1.62-1.74 (m, 3H), 1.54 (s, 3H, CH₃), 1.42-1.47 (m, 1H), 1.36 (s, 3H, CH₃), 1.18 (s, 6H, CH₃), 0.93 (t, J=8.0 Hz, 9H, SiCH₂CH₃), 0.92 (s, 9H, (CH₃)₃C), 0.80 (d, J=6.4 Hz, 3H, CHCH₃), 0.54 (q, J=8.0 Hz, 6H, SiCH₂), 0.11 (s, 3H, SiCH₃), 0.10 (s, 3H, SiCH₃).

¹³C NMR (100 MHz, CDCl₃) δ 146.2, 145.7, 142.8, 132.4, 128.2, 127.5, 126.1, 124.6, 122.3, 114.8, 108.8, 77.2, 74.2, 73.3, 69.8, 54.1, 54.1, 44.6, 43.7, 38.1, 36.4, 35.8, 30.6, 30.0, 27.8, 27.6, 26.0, 25.4, 18.4, 15.8, 7.1, 6.8, −4.5, −4.6.
TLC analysis: R_{f}=0.7 (hexane:ethyl acetate=10:1 (v/v))

### Synthesis Example 28-2

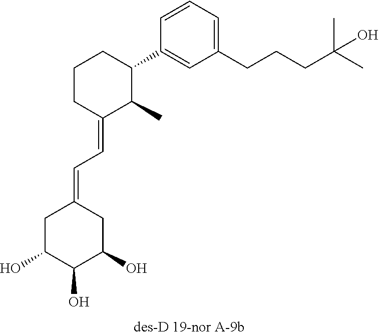

A 1.0 M THF solution of n-tetrabutylammonium fluoride (1.7 mL, 1.7 mmol) was added at room temperature to a solution of compound des-D 19-nor A-7b (290 mg, 0.42 mmol) in THF (0.4 mL) and stirred for 15 hours. After a saturated aqueous ammonium chloride solution was added to the reaction mixture, the resulting mixture was extracted with diethyl ether. The organic layer was dried over anhydrous magnesium sulfate and then filtered. The filtrate was concentrated to give a crude product, to which methanol (4.2 nL) was then added. To the mixture was added at 0° C. 1 M hydrochloric acid (0.15 mL) and stirred at room temperature for 5 hours. After a saturated aqueous sodium bicarbonate solution was added to the reaction mixture, the resulting mixture was extracted with ethyl acetate. The organic layer was dried over anhydrous magnesium sulfate and then filtered. The filtrate was concentrated, and the resulting residue was purified by silica gel column chromatography to give compound des-D 19-nor A-9b (176 mg) in a yield of 98%.

¹H NMR (400 MHz, CD₃OD) δ 7.09 (t, J=8.0 Hz, 1H, Ar), 6.68-6.94 (m, 3H, Ar), 6.20 (d, J=10.8 Hz, 1H, C═CH), 6.01 (d, J=10.8 Hz, 1H, C═CH), 3.83 (br s, 1H, CHO), 3.38-3.49 (m, 2H, CHO), 2.85-2.94 (m, 1H, ArH), 2.47-2.68 (m 1H), 2.51 (t, J=7.6 Hz, 2H, ArCH₂), 2.03-2.45 (m, 5H), 1.54-1.88 (m, 6H), 1.25-1.41 (m, 3H), 1.07 (s, 6H, CCH₃), 0.73 (d, J=6.4 Hz, 3H, CHCH₃).

¹³C NMR (100 MHz, CD₃OD) δ 147.8, 146.8, 144.1, 133.7, 129.6, 128.9, 127.5, 126.1, 123.7, 116.5, 74.3, 72.4, 72.0, 71.6, 45.1, 44.6, 40.6, 37.7, 37.3, 32.2, 31.1, 29.5, 29.1, 27.8, 16.7.
TLC analysis: R_{f}=0.4 (ethyl acetate:methanol=10:1 (v/v))

### Synthesis Example 28-3

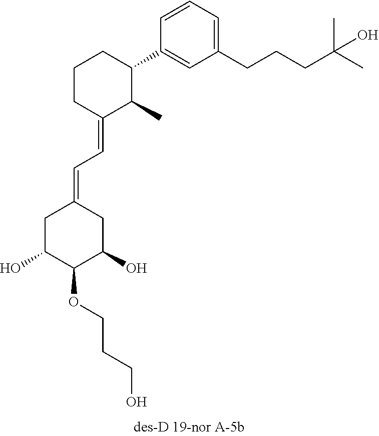

Similar to the synthesis of compound A-1 from compound A-0, a crude product of compound des-D 19-nor A-10b was synthesized from compound des-D 19-nor A-9b (128 mg, 0.30 mmol). To a solution of the crude product in tetrahydrofuran (1.0 mL) was added at 0° C. a 0.5 M THF solution of 9-BBN (3.0 mL, 1.5 mmol) and stirred at room temperature for 5 hours. To the mixture was added at 0° C. a 1 M aqueous NaOH solution (0.75 mL) and a 30% hydrogen peroxide solution (0.20 mL) and stirred for 30 minutes. After a saturated aqueous sodium thiosulfate solution (0.5 mL) was added to the reaction mixture, the resulting mixture was extracted with diethyl ether. The organic layer was washed with a saturated aqueous sodium chloride solution and then dried over anhydrous magnesium sulfate. After the organic layer was filtered, the filtrate was concentrated. The resulting residue was purified by silica gel column chromatography to give compound des-D A-5b (16 mg) in a yield of 11%.

¹H NMR (600 MHz, CDCl₃) δ 7.20 (t, J=7.2 Hz, 1H, Ar), 7.96-7.02 (m, 3H, Ar), 6.37 (d, J=10.8 Hz, 1H, C═CH), 6.08 (d, J=10.8 Hz, 1H, C═CH), 3.77-3.95 (m, 7H), 2.97 (br d, J=13.2 Hz, 1H, ArCH), 2.65-2.70 (m, 1H), 2.61 (t, J=7.2 Hz, 2H, ArCH₂), 2.29-2.53 (m, 3H), 2.18-2.23 (m, 1H), 1.40-2.07 (m, 16H), 1.20 (s, 6H, CCH₃), 0.83 (d, J=6.0 Hz, 3H, CHCH₃).

### Synthesis Example 29

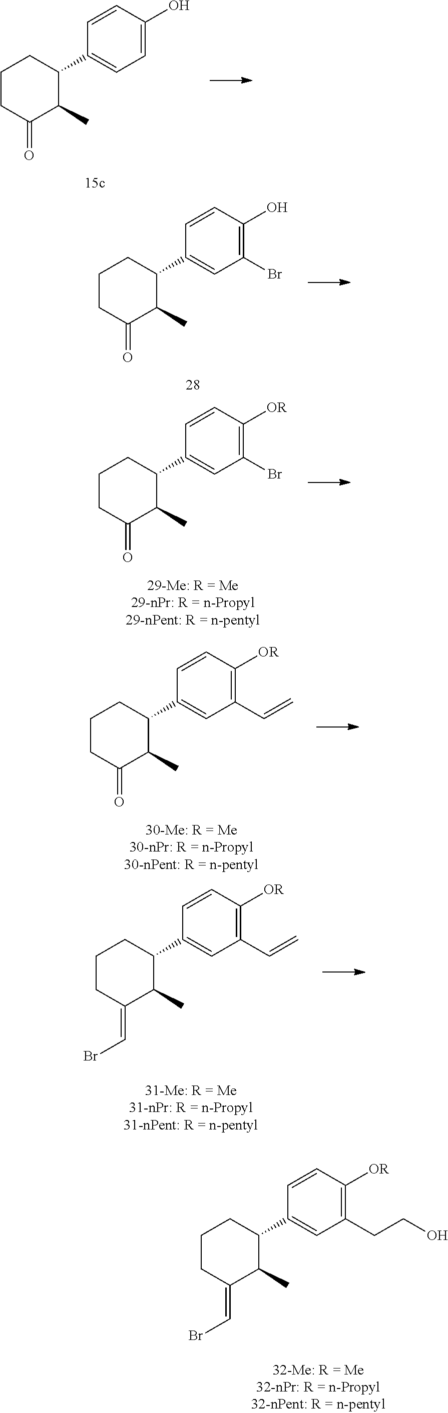

### Synthesis Example 29-1

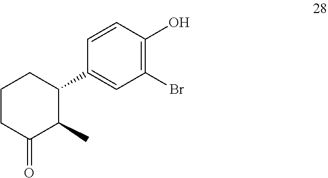 [SMILES: C[C@H]1C(=O)CCC[C@@H]1c1ccc(O)c(Br)c1]

A solution of compound 15c (2.0 g, 9.8 mmol, 1.0 equivalent) in methylene chloride (20 mL) was cooled to −40° C., to which N-bromosuccinimide (1.7 g, 9.8 mmol, 1.0 equivalent) was then added in two parts. After the mixture was stirred for 3 hours, the reaction was quenched by adding a saturated sodium hydrogen carbonate solution, followed by extraction with diethyl ether. The resulting organic layer was washed with a 10% aqueous sodium thiosulfate solution and a saturated aqueous sodium chloride solution, then dried over anhydrous sodium sulfate, and then filtered, and the filtrate was concentrated under reduced pressure. The resulting residue was purified by silica gel column chromatography (hexane/ethyl acetate) to give compound 28 (0.90 g) in a yield of 32%.

¹H NMR (600 MHz, CDCl₃) δ 7.30 (br s, 1H, Ar), 7.06 (d, J=7.8 Hz, 1H, Ar), 6.98 (d, J=7.8 Hz, 1H, Ar), 2.41-2.57 (m, 4H), 2.12-2.17 (m, 1H), 1.94-1.99 (m, 1H), 1.89 (dq, J=4.5, 8.4 Hz, 1H), 1.74 (tq, J=4.2, 9.0 Hz, 1H), 0.82 (d, J=6.6 Hz, 3H, CH₃).

¹³C NMR (150 MHz, CDCl₃) δ 212.0, 150.9, 137.7, 130.4, 127.9, 116.1, 110.2, 52.1, 50.7, 41.7, 34.6, 26.3, 12.2.

### Synthesis Example 29-2

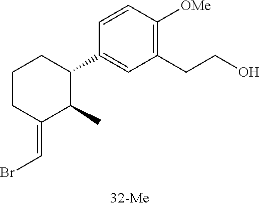 [SMILES: COc1ccc([C@H]2CCC/C(=C\Br)[C@@H]2C)cc1CCO]

Iodomethane (0.18 mL, 2.84 mmol, 2.0 equivalents) was added at 0° C. to a solution of compound 28 (0.40 g, 1.42 mmol, 1.0 equivalent) and potassium carbonate (0.59 g, 4.26 mmol, 3.0 equivalents) in N,N-dimethylformamide (1.4 mL). After the mixture was stirred at 50° C. for 3 hours, the reaction was quenched by adding water at 0° C., followed by extraction with diethyl ether. The resulting organic layer was washed with a saturated aqueous sodium chloride solution, then dried over anhydrous sodium sulfate, and then filtered. The filtrate was concentrated under reduced pressure to give a crude product of compound 29-Me.

Under an argon atmosphere, toluene (5.5 mL) and water (1.8 mL) were added to a mixture of palladium(II) acetate (32 mg, 0.14 mmol, 10 mol %), triphenylphosphine (73 mg, 0.28 mmol, 20 mol %), 2,4,6-trivinylboroxin-pyridine complex (0.34 g, 1.42 mmol, 1.0 equivalent), the crude product of compound 29-Me, and potassium carbonate (0.2 g, 1.42 mmol, 1.0 equivalent). After the resulting mixture was stirred at 80° C. for 15 hours, the reaction mixture was extracted with diethyl ether. The organic layer was washed with water and a saturated aqueous sodium chloride solution. The organic layer was dried over anhydrous sodium sulfate and then filtered. The filtrate was concentrated under reduced pressure to give a crude product of compound 30-Me.

Under an argon atmosphere, a solution of (bromomethyl)triphenylphosphonium bromide (1.86 g, 4.26 mmol, 3.0 equivalents) in tetrahydrofuran (2.8 mL) was cooled to −10° C., and a 0.56 M tetrahydrofuran solution of lithium diisopropylamide (7.1 mL, 4.0 mmol, 2.9 equivalents) was added to the solution. While being heated at 0° C., the mixture was stirred for 30 minutes. The mixture was then cooled to −40° C., to which a solution of the crude product of compound 30-Me in tetrahydrofuran (7.1 mL) was added. While being heated at 0° C., the resulting mixture was stirred for 2 hours. The reaction mixture was filtered through silica gel with hexane. The resulting filtrate was concentrated under reduced pressure to give a crude product of compound 31-Me.

Under an argon atmosphere, a 0.5 M tetrahydrofuran solution of 9-BBN (3.6 mL, 1.8 mmol, 1.27 equivalents) was added at 0° C. to a solution of the crude product of compound 31-Me in tetrahydrofuran (14 mL). After being stirred at room temperature for 7 hours, the mixture was cooled to 0° C., to which a 1 M aqueous sodium hydroxide solution (9.5 mL) and a 30% aqueous hydrogen peroxide solution (2.0 mL) were then added. After the mixture was stirred at room temperature for 1 hour, the reaction was quenched by adding a 10% aqueous sodium thiosulfate solution at 0° C., followed by extraction with diethyl ether. The organic layer was washed with a saturated aqueous sodium chloride solution. The organic layer was dried over anhydrous sodium sulfate and then filtered. The filtrate was concentrated under reduced pressure. The concentrate was purified by silica gel column chromatography (hexane/ethyl acetate) to give compound 32-Me (0.25 g) in a yield of 53%.

¹H NMR (400 MHz, CDCl₃) δ 7.00 (dd, J=2.0, 8.4 Hz, 1H, Ar), 6.95 (d, J=2.4 Hz, 1H, Ar), 6.80 (d, J=8.4 Hz, 1H, Ar), 5.92 (t, J=1.6 Hz, 1H, C═CBrH), 3.82 (s, 3H, OCH₃), 3.79-3.84 (m, 2H, CH₂O), 3.09 (br d, J=15.2 Hz, 1H, ArCH), 2.89 (t, J=6.8 Hz, 2H, ArCH₂), 2.23-2.33 (m, 1H), 2.16 (dt, J=3.6, 12 Hz, 1H), 1.83-1.96 (m, 3H), 1.55-1.74 (m, 2H), 0.81 (d, J=6.4 Hz, 3H, CH₃CH).

¹³C NMR (100 MHz, CDCl₃) δ 156.0, 148.5, 137.5, 129.8, 126.9, 126.4, 110.4, 98.9, 63.0, 55.4, 52.4, 44.0, 35.6, 34.3, 32.4, 26.6, 15.7.

### Synthesis Example 29-3

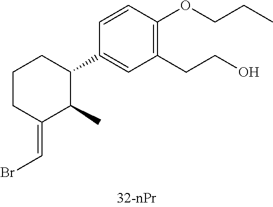 [SMILES: CCCOc1ccc([C@H]2CCC/C(=C\Br)[C@@H]2C)cc1CCO]

Similar to the synthesis of compound 32-Me from compound 28, compound 32-nPr (0.161 g) was synthesized in a yield of 29% from compound 28 (0.428 g, 1.51 mmol).

¹H NMR (400 MHz, CDCl₃) δ 6.97 (dd, J=2.0, 8.0 Hz, 1H, Ar), 6.95 (t, J=2.0 Hz, 1H, Ar), 6.78 (d, J=8.8 Hz, 1H, Ar), 5.92 (br s, 1H, C═CBrH), 3.92 (t, J=6.4 Hz, 2H, ArOCH₂), 3.80-3.87 (m, 2H, CH₂OH), 3.09 (br d, J=14.0 Hz, 1H, ArCH), 2.90 (t, J=6.4 Hz, 2H, ArCH₂), 2.24-2.27 (m, 1H), 2.10-2.17 (m, 1H), 1.76-1.94 (m, 6H), 1.58-1.66 (m, 1H), 1.37-1.48 (m, 1H), 1.05 (t, J=7.6 Hz, 3H, CH₂CH₃), 0.81 (d, J=6.8 Hz, 3H, CHCH₃).

¹³C NMR (100 MHz, CDCl₃) δ 155.5, 148.5, 137.3, 129.8, 127.1, 126.3, 111.2, 98.9, 69.6, 63.1, 52.5, 44.0, 35.6, 34.5, 32.4, 26.6, 22.7, 15.7, 10.7.

### Synthesis Example 29-4

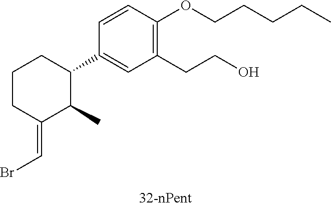 [SMILES: CCCCCOc1ccc([C@H]2CCC/C(=C\Br)[C@@H]2C)cc1CCO]

Similar to the synthesis of compound 32-Me from compound 28, compound 32-nPent (0.175 g) was synthesized in a yield of 31% from compound 28 (0.408 g, 1.44 mmol).

¹H NMR (400 MHz, CDCl₃) δ 6.97 (dd, J=2.4, 8.4 Hz, 1H, Ar), 6.94 (d, J=2.4 Hz, 1H, Ar), 6.78 (d, J=8.0 Hz, 1H, Ar), 5.92 (s, C═CBrH), 3.95 (t, J=6.4 Hz, 2H, ArOCH₂), 3.84 (dt, J=5.2, 6.4 Hz, 2H, CH₂OH), 3.09 (br d, J=14.0 Hz, 1H, ArCH), 2.90 (t, J=6.4 Hz, 2H, ArCH₂), 2.24-2.33 (m, 1H), 2.15 (dt, J=3.6, 11.2 Hz, 1H), 1.59-1.95 (m, 8H), 1.33-1.50 (m, 5H), 0.93 (t, J=7.2 Hz, 3H, CH₂CH₃), 0.81 (d, J=6.4 Hz, 3H, CHCH₃).

### 13C NMR (100 MHz, CD₃OD) δ 155.5, 148.5, 137.3, 129.8, 127.1, 126.3, 111.1, 98.9, 68.0, 63.1, 52.5, 44.0, 35.6, 34.5, 32.4, 29.0, 28.3, 26.6, 22.4, 15.7, 14.0.

### Synthesis Example 30

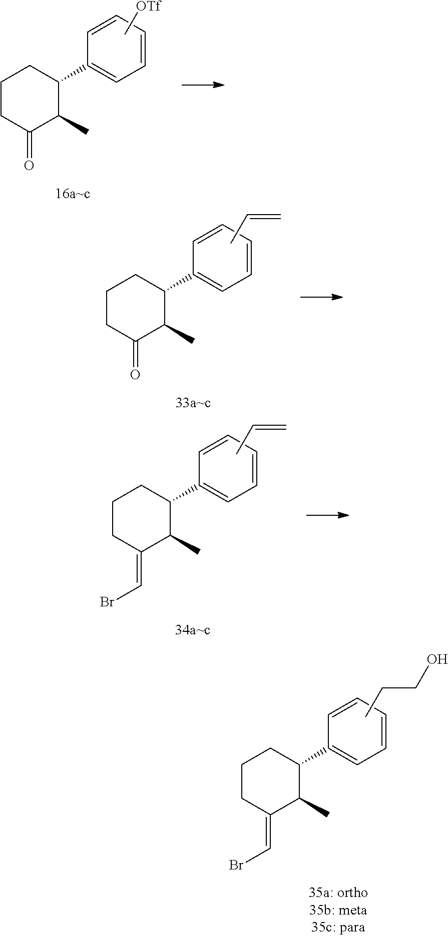

### Synthesis Example 30-1

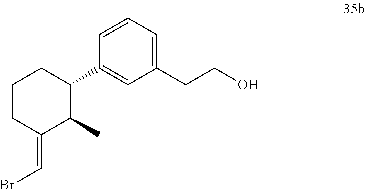 [SMILES: C[C@H]1/C(=C/Br)CCC[C@@H]1c1cccc(CCO)c1]

Under an argon atmosphere, a solution of compound 16b (0.336 g, 1.0 mmol, 1.0 equivalent) in toluene (3.8 mL) and water (3.8 mL) were added to a mixture of palladium(II) acetate (22.5 mg, 0.1 mmol, 10 mol %), triphenylphosphine (52.5 mg, 0.2 mmol, 20 mol %), 2,4,6-trivinylboroxin-pyridine complex (0.24 g, 1.0 mmol, 1.0 equivalent), and potassium carbonate (0.14 g, 1.0 mmol, 1.0 equivalent). After the mixture was stirred at 80° C. for 15 hours, the reaction mixture was extracted with diethyl ether. The organic layer was washed with water and a saturated aqueous sodium chloride solution. The organic layer was dried over anhydrous sodium sulfate and then filtered. The filtrate was concentrated under reduced pressure to give a crude product of compound 33b.

Under an argon atmosphere, a solution of (bromomethyl)triphenylphosphonium bromide (0.87 g, 2.0 mmol, 2.0 equivalents) in tetrahydrofuran (2.0 mL) was cooled to −20° C., to which a 1.0 M tetrahydrofuran solution of lithium diisopropylamide (2.0 mL, 2.0 mmol, 2.0 equivalents) was then added. While being heated at 0° C., the mixture was stirred for 30 minutes. After the mixture was cooled to −40° C., a solution of the crude product of compound 33b in tetrahydrofuran (5.0 mL) was added to the mixture. The resulting mixture was stirred for 12 hours while being heated at 0° C. The reaction mixture was filtered through silica gel with hexane. The resulting filtrate was concentrated under reduced pressure to give a crude product of compound 34b.

Under an argon atmosphere, a 0.5 M tetrahydrofuran solution of 9-BBN (4.0 mL, 4.0 mmol, 2.0 equivalents) was added at 0° C. to a solution of the crude product of compound 34b in tetrahydrofuran (10 mL). After being stirred at room temperature for 5 hours, the mixture was cooled to 0° C., to which a 1M aqueous sodium hydroxide solution (6.7 mL) and a 30% aqueous hydrogen peroxide solution (1.4 mL) were then added. After the mixture was stirred at room temperature for 1 hour, the reaction was quenched by adding a 10% aqueous sodium thiosulfate solution at 0° C., followed by extraction with diethyl ether. The organic layer was washed with a saturated aqueous sodium chloride solution. The organic layer was dried over anhydrous sodium sulfate and then filtered. The filtrate was concentrated under reduced pressure, and the concentrate was purified by silica gel column chromatography (hexane/ethyl acetate) to give compound 35b (0.149 g) in a yield of 48%.

¹H NMR (400 MHz, CDCl₃) δ 7.22-7.26 (m, 1H, Ar), 7.01-7.08 (m, 3H, Ar), 5.93 (s, 1H, C═CBrH), 3.86 (t, J=6.4 Hz, 2H, CH₂O), 3.10 (br d, J=14.4 Hz, 1H, ArCH), 2.86 (t, J=6.4 Hz, 2H, ArCH₂), 2.31-2.38 (m, 1H), 2.23 (dt, J=3.6, 11.6 Hz, 1H), 1.86-1.96 (m, 3H), 1.40-1.73 (m, 2H), 0.81 (d, J=6.4 Hz, 3H, CHCH₃).

¹³C NMR (100 MHz, CDCl₃) δ 148.3, 145.6, 138.5, 128.7, 128.1, 126.9, 125.4, 99.0, 63.6, 53.2, 43.7, 39.2, 35.4, 32.4, 26.6, 15.7.

### Synthesis Example 31

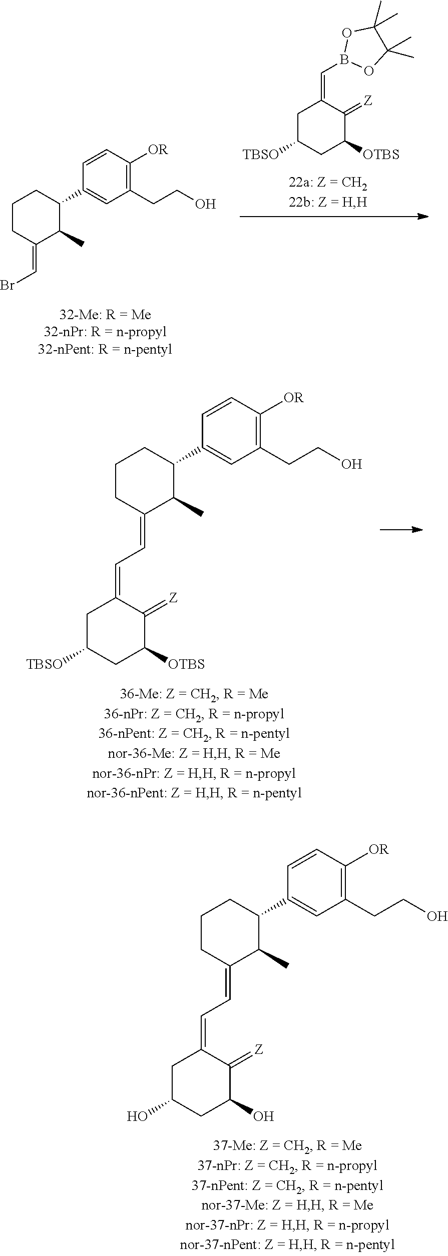

### Synthesis Example 31-1

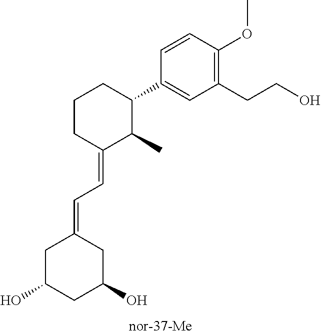 [SMILES: COc1ccc([C@H]2CCC/C(=C\C=C3C[C@@H](O)C[C@H](O)C3)[C@@H]2C)cc1CCO]

Under an argon atmosphere, a solution of compound 32-Me (0.18 g, 0.53 mmol, 1.0 equivalent) and compound 22b (0.28 g, 0.58 mmol, 1.1 equivalents) in tetrahydrofuran (5.3 mL) and a 3 N potassium hydroxide solution (0.35 mL) were added to 1,1′-bis(diphenylphosphino)ferrocene-palladium(II) dichloride (31.0 mg, 42 μmol, 8.0 mol %) and stirred at 50° C. for 22 hours. After being cooled to room temperature, the reaction solution was dried over anhydrous sodium sulfate and then filtered through Celite with diethyl ether. The resulting filtrate was concentrated under reduced pressure to give a crude product of compound nor-36-Me.

Under an argon atmosphere, a 1.0 M tetrahydrofuran solution of tetrabutylammonium fluoride (2.65 mL, 2.65 mmol, 5.0 equivalents) was added at 0° C. to a solution of the crude product of compound nor-36-Me in tetrahydrofuran (0.53 mL). After the mixture was stirred at room temperature for 20 hours, the reaction was quenched with a saturated ammonium chloride solution, followed by extraction with diethyl ether. The resulting organic layer was dried over anhydrous sodium sulfate and then filtered, and the filtrate was concentrated. The resulting residue was purified by silica gel column chromatography (hexane/ethyl acetate/methanol) to give compound nor-37-Me (0.138 g) in a yield of 68%.

¹H NMR (400 MHz, CD₃OD) δ 6.93 (d, J=8.0 Hz, 1H, Ar), 6.91 (s, 1H, Ar), 6.77 (d, J=8.0 Hz, 1H, Ar), 6.18 (d, J=10.8 Hz, 1H, C═CH), 6.04 (d, J=10.8 Hz, 1H, C═CH), 3.90-4.01 (m, 2H, CHO), 3.73 (s, 3H, CH₃O), 3.61 (t, J=7.6 Hz, 2H, CH₂OH), 2.91 (br d, J=13.6 Hz, 1H, ArCH), 2.75 (t, J=7.6 Hz, ArCH₂), 2.54 (br d, J=13.2 Hz, 1H), 2.37 (br d, J=9.6 Hz, 1H), 2.00-2.26 (m, 4H), 1.56-1.87 (m, 6H), 1.25-1.33 (m, 1H), 0.74 (d, J=6.8 Hz, 3H, CH₃CH).

¹³C NMR (100 MHz, CD₃OD) δ 157.7, 146.3, 139.9, 135.1, 131.0, 128.1, 127.7, 124.1, 116.5, 111.7, 68.3, 68.0, 63.3, 56.1, 55.0, 45.9, 45.4, 43.0, 38.0, 37.4, 35.4, 31.1, 29.1, 16.7.

### Synthesis Example 31-2

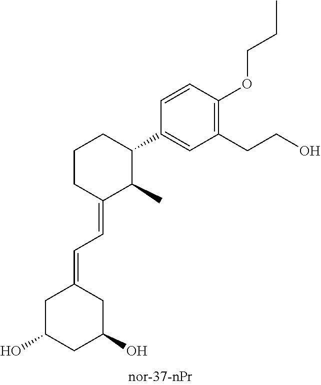 [SMILES: CCCOc1ccc([C@H]2CCC/C(=C\C=C3C[C@@H](O)C[C@H](O)C3)[C@@H]2C)cc1CCO]

Similar to the synthesis of compound nor-37-Me from compound 32-Me, compound nor-37-nPr (52.4 mg) was synthesized in a yield of 79% from compound 32-nPr (61 mg, 0.16 mmol) and compound 22b (85 mg, 0.176 mmol).

¹H NMR (400 MHz, CDCl₃) δ 6.98 (d, J=7.6 Hz, 1H, Ar), 6.97 (s, 1H, Ar), 6.78 (d, J=7.6 Hz, 1H, Ar), 6.34 (d, J=10.8 Hz, 1H, C═CH), 6.08 (d, J=10.8 Hz, 1H, C═CH), 4.03-4.16 (m, 2H, CHO), 3.93 (t, J=6.4 Hz, 2H, ArOCH₂), 3.84 (t, J=6.4 Hz, 2H, CH₂OH), 2.70-2.90 (m, 1H, ArCH), 2.91 (t, J=6.4 Hz, 2H, ArCH₂), 2.75 (br d, J=9.2 Hz, 1H), 2.51 (br d, J=13.2 Hz, 1H), 2.10-2.32 (m, 3H), 1.30-1.99 (m, 13H), 1.05 (t, J=7.6 Hz, 3H, CH₂CH₃), 0.84 (d, J=6.4 Hz, 3H, CHCH₃).

¹³C NMR (100 MHz, CDCl₃) δ 155.3, 146.5, 138.4, 132.3, 129.8, 126.9, 126.4, 124.0, 114.5, 111.1, 69.5, 67.4, 67.2, 63.1, 52.9, 44.8, 43.9, 42.2, 37.2, 35.8, 34.5, 29.8, 27.5, 22.7, 16.1, 10.7.

### Synthesis Example 31-3

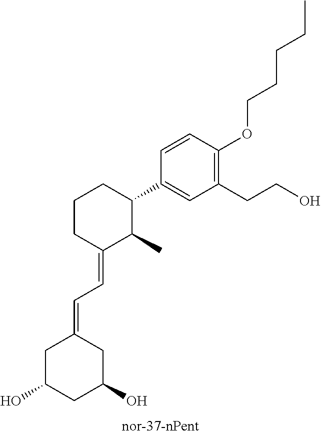 [SMILES: CCCCCOc1ccc([C@H]2CCC/C(=C\C=C3C[C@@H](O)C[C@H](O)C3)[C@@H]2C)cc1CCO]

Similar to the synthesis of compound nor-37-Me from compound 32-Me, compound nor-37-nPent (153 mg) was synthesized in a yield of 91% from compound 32-nPent (152 mg, 0.38 mmol) and compound 22b (200 mg, 0.418 mmol).

¹H NMR (400 MHz, CDCl₃) δ 6.89 (d, J=8.0 Hz, 1H, Ar), 6.97 (s, 1H, Ar), 6.78 (d, J=8.0 Hz, 1H, Ar), 6.34 (d, J=10.8 Hz, 1H, C═CH), 6.08 (d, J=10.8 Hz, 1H, C═CH), 4.05-4.17 (m, 2H, CHO), 3.95 (t, J=6.4 Hz, 2H, ArOCH₂), 3.84 (t, J=6.4 Hz, 2H, CH₂OH), 2.88-2.93 (m, 1H, ArCH), 2.90 (t, J=6.4 Hz, 2H, ArCH₂), 2.75 (br d, J=9.6 Hz, 1H), 2.51 (br d, J=10.0 Hz, 1H), 2.12-2.32 (m, 3H), 1.30-1.98 (m, 17H), 0.94 (t, J=6.8 Hz, 3H, CH₂CH₃), 0.84 (d, J=6.4 Hz, 3H, CHCH₃).

¹³C NMR (100 MHz, CDCl₃) δ 155.4, 146.5, 138.4, 132.3, 129.9, 126.9, 126.4, 124.0, 114.6, 111.1, 68.1, 67.4, 67.2, 63.1, 52.9, 44.8, 44.0, 42.2, 37.2, 35.8, 34.5, 29.8, 29.1, 28.4, 27.5, 22.4, 16.1, 14.0.

### Synthesis Example 32

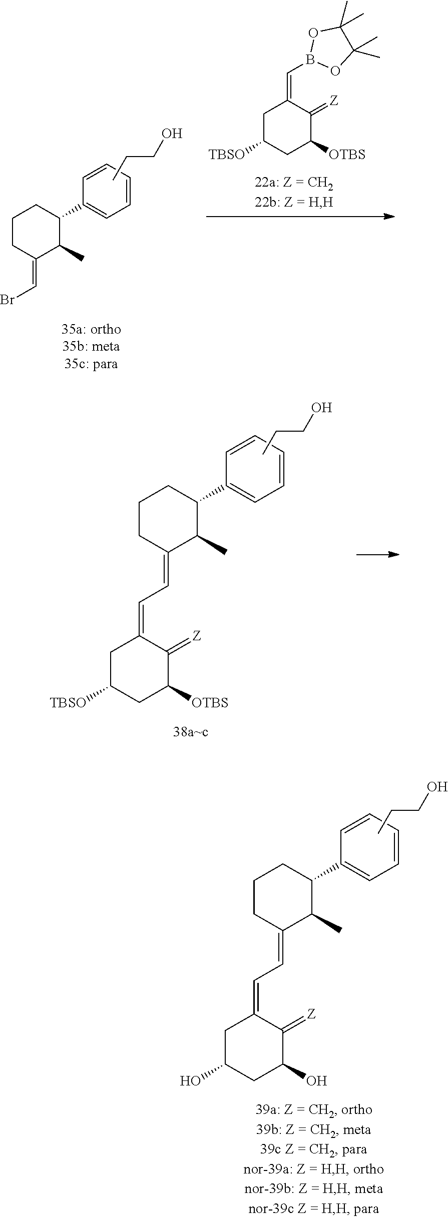

### Synthesis Example 32-1

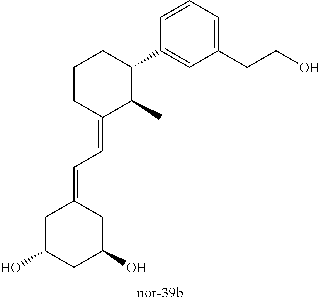 [SMILES: C[C@H]1/C(=C/C=C2C[C@@H](O)C[C@H](O)C2)CCC[C@@H]1c1cccc(CCO)c1]

Under an argon atmosphere, a solution of compound 35b (0.12 g, 0.387 mmol, 1.0 equivalent) and compound 22b (0.205 g, 0.43 mmol, 1.1 equivalents) in tetrahydrofuran (3.9 mL) and a 3 N potassium hydroxide solution (0.26 mL) were added to 1,1′-bis(diphenylphosphino)ferrocene-palladium(II) dichloride (23.0 mg, 31.4 μmol, 8.0 mol %) and stirred at 50° C. for 3.5 hours. After being cooled to room temperature, the reaction solution was dried over anhydrous sodium sulfate and then filtered through Celite with diethyl ether. The resulting filtrate was concentrated under reduced pressure to give a crude product of compound 38b.

Under an argon atmosphere, a 1.0 M tetrahydrofuran solution of tetrabutylammonium fluoride (1.9 mL, 1.94 mmol, 5.0 equivalents) was added at 0° C. to the crude product of compound 38b. After the mixture was stirred at room temperature for 20 hours, the reaction was quenched with a saturated ammonium chloride solution, followed by extraction with diethyl ether. The resulting organic layer was dried over anhydrous sodium sulfate and then filtered, and the filtrate was concentrated. The resulting residue was purified by silica gel column chromatography (hexane/ethyl acetate/methanol) to give compound nor-38b (0.115 g) in a yield of 84%.

¹H NMR (400 MHz, CD₃OD) δ 7.14 (t, J=9.2 Hz, 1H, Ar), 6.89-7.01 (m, 3H, Ar), 6.19 (d, J=10.8 Hz, 1H, C═CH), 6.04 (d, J=10.8 Hz, 1H, C═CH), 3.90-4.00 (m, 2H, CHO), 3.66 (t, J=7.2 Hz, 2H, CH₂O), 2.92 (br d, J=13.6 Hz, 1H, ArH), 2.73 (t, J=7.2 Hz, 2H, ArCH₂), 2.54 (dd, J=4.0, 13.6 Hz, 1H), 2.34 (dd, J=3.6, 14.0 Hz, 1H), 2.23-2.33 (m, 1H), 2.20 (dd, J 8.0, 14.0 Hz, 1H), 2.06-2.14 (m, 2H, 1.65-1.88 (m, 10H), 0.74 (d, J=6.4 Hz, 3H, CH₃).

### 13C NMR (100 MHz, CD₃OD) δ 148.1, 146.1, 140.6, 135.2, 129.7, 129.5, 128.0, 126.6, 124.1, 116.6, 68.3, 38.0, 64.7, 55.8, 45.9, 45.1, 43.0, 40.6, 37.9, 37.3, 31.1, 29.1, 16.7.

### Synthesis Example 33 (Synthesis of Intermediate Compound with R¹ Being Ethyl)

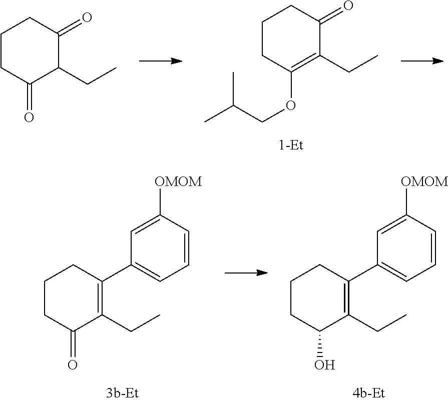

### Synthesis Example 33-1

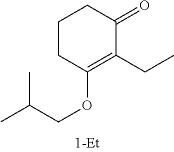

Similar to the synthesis of compound 1 from 2-methylcyclohexane-1,3-dione, compound 1-Et (1.30 g) was synthesized in a yield of 83% from 2-ethylcyclohexane-1,3-dione (1.12 g, 8.0 mmol).

¹H NMR (400 MHz, CDCl₃) δ 3.75 (d, J=6.4 Hz, 2H, CH₂O), 2.54 (t, J=6.2 Hz, 2H, CH₂), 2.26-2.35 (m, 4H), 1.93-2.05 (m, 3H), 1.00 (d, J=6.8 Hz, 6H, CH₃CH), 0.93 (t, J=7.2 Hz, 3H, CH₂CH₃).

¹³C NMR (100 MHz, CDCl₃) δ 198.2, 171.4, 121.2, 73.8, 36.4, 28.8, 25.4, 21.0, 19.0, 15.4, 13.3.

### Synthesis Example 33-2

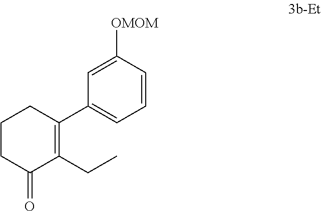

Similar to the synthesis of compound 3a from compound 1, compound 3b-Et (0.30 g) was synthesized in a yield of 29% from compound 1-Et (0.785 g, 4.0 mmol).

¹H NMR (600 MHz, CDCl₃) δ 7.30 (t, J=7.8 Hz, 1H, Ar), 7.00 (d, J=8.4 Hz, Ar), 6.87 (s, 1H, Ar), 6.82 (d, J=7.8 Hz, 1H, Ar), 5.19 (s, 2H, OCH₂O), 3.50 (s, 3H, CH₃O), 2.58 (t, J=6.0 Hz, 2H, CH₂), 2.50 (t, J=6.6 Hz, 2H, CH₂), 2.15 (q, J=7.2 Hz, 2H, CH₂CH₃), 2.05-2.10 (m, 2H, CH₂), 0.91 (t, J=7.2 Hz, CH₃CH₂).

¹³C NMR (150 MHz, CDCl₃) δ 199.5, 157.2, 156.3, 142.9, 137.9, 129.5, 120.1, 115.4, 114.6, 94.5, 56.1, 38.2, 33.3, 22.8, 20.0, 14.3.

### Synthesis Example 33-3

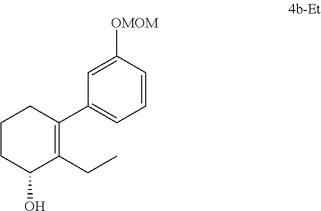

Similar to the synthesis of compound 4a from compound 3a, compound 4b-Et (0.224 g) was synthesized in a yield of 74% from compound 3b-Et (0.303 g, 1.16 mmol).

¹H NMR (600 MHz, CDCl₃) δ 7.23 (t, J=7.2 Hz, 1H, Ar), 6.92 (dd, J=2.4, 7.2 Hz, 1H, Ar), 6.81 (s, 1H, Ar), 6.77 (d, J=7.8 Hz, 1H, Ar), 5.17 (s, 2H, OCH₂O), 4.30 (br s, 1H, CHO), 3.49 (s, 3H, OCH₃), 2.00-2.30 (m, 4H, 1.64-1.90 (m, 4H), 1.45 (d, J=7.2 Hz, 1H, OH), 0.95 (t, J=7.2 Hz, 3H, CH₃CH₂).

¹³C NMR (150 MHz, CDCl₃) δ 157.1, 145.0, 136.8, 136.2, 129.1, 121.5, 115.8, 114.2, 94.5, 65.9, 56.0, 32.6, 32.0, 23.6, 18.2, 13.7.

### Synthesis Example 34 (Method Alternative to Synthesis Example 3)

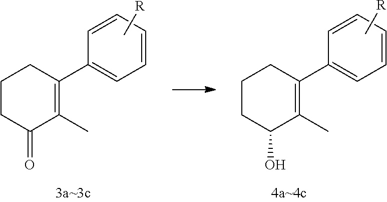

### Synthesis Example 34-1

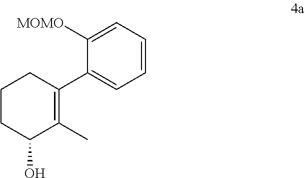

Under an argon atmosphere, a 0.5 M toluene solution of (S)-5,5-diphenyl-2-methyl-3,4-propano-1,3,2-oxazaborolidine (0.4 mL, 0.2 mmol, 20.0 mol %) and compound 3a (0.25 g, 1.0 mmol) were dissolved in toluene (1.0 mL) at −40° C. After a 0.2 M toluene solution of catecholborane (6.0 mL, 1.2 mmol, 1.2 equivalents) was added dropwise to the reactant solution over 5 hours, the mixture was stirred at −40° C. for 12 hours. The reaction mixture was quenched by adding a 1 M sodium hydroxide solution, followed by extraction with diethyl ether. The resulting organic layer was washed with a saturated aqueous sodium chloride solution, then dried over anhydrous sodium sulfate, and then filtered, and the filtrate was concentrated. The residue was purified by silica gel column chromatography (hexane/ethyl acetate) to give compound (R)-4a (0.24 g) in a yield of 97%.

(R)-4a (optical purity; 91% ee): Enantiomeric excess determined by a normal-phase HPLC [2.1 mmI.D.×250 mmL, CHIRALPAK (registered trademark) AY-H column, 0.2 mL/min, Hexane/EtOH (96:4) solvent system, 25° C., λ=254 nm, (R)-4a: t_{R}=7.9 min, (S)-4a: t_{R}=9.3 min].

### Synthesis Example 34-2

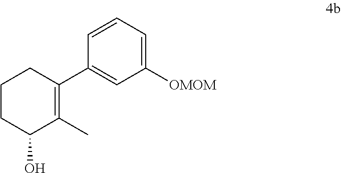

Similar to the synthesis of compound (R)-4a from compound 3a, compound (R)-4b (0.25 g) was synthesized in a yield of >99% from compound 3b (0.25 g, 1.0 mmol).

(R)-4b (optical purity; 95% ee): Enantiomeric excess determined by a normal-phase HPLC [2.1 mmI.D.×250 mmL, CHIRALPAK AY-H column, 0.2 mL/min, Hexane/EtOH (96:4) solvent system, 25° C., λ=254 nm, (R)-4a: t_{R}=8.3 min, (S)-4a: t_{R}=9.5 min].

### Synthesis Example 34-3

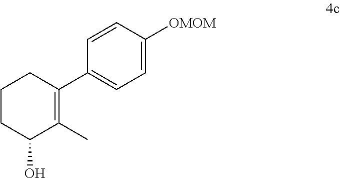

Similar to the synthesis of compound (R)-4a from compound 3a, compound (R)-4c (0.23 g) was synthesized in a yield of 93% from compound 3c (0.25 g, 1.0 mmol).

(R)-4c (optical purity; 90% ee): Enantiomeric excess determined by a normal-phase HPLC [2.1 mmI.D.×250 mmL, CHIRALPAK AY-H column, 0.2 mL/min, Hexane/EtOH (95:5) solvent system, 25° C., λ=254 nm, (R)-4a: t_{R}=7.7 min, (S)-4a: t_{R}=9.8 min].

### Synthesis Example 35: Synthesis of Racemic Compound 15b and Separation Method Using Preparative Chiral Column for Separation of Optically Active Compounds

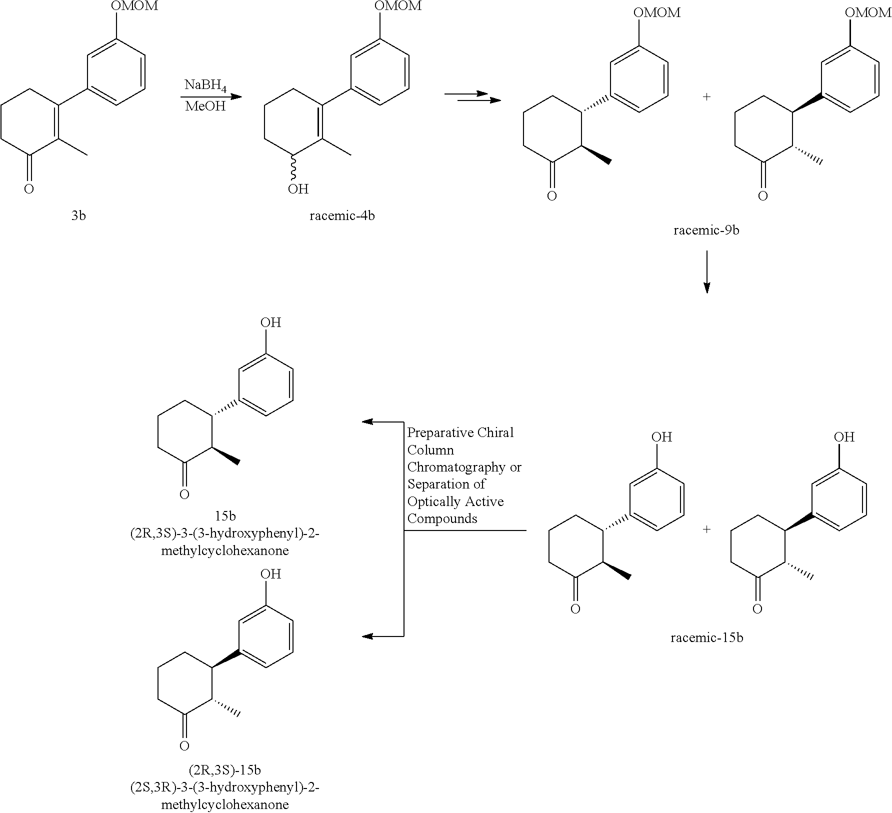

### Synthesis Example 35-1

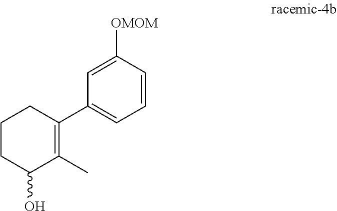

Compound 3b (1.23 g, 5.0 mmol) was slowly added at 0° C. to a mixture of NaBH₄ (264 mg, 7.0 mmol) and methanol (35 mL). After the reactant liquid was stirred at room temperature for 1 hour, water (30 mL) was slowly added to the reaction mixture. The resulting mixture was extracted with ethyl acetate, and the organic layer was washed with a saturated aqueous sodium chloride solution. The organic layer was dried over anhydrous magnesium sulfate and then filtered. The filtrate was concentrated to give racemic compound 4b (1.17 g) in a yield of 94%. The NMR spectrum of the resulting compound was consistent with that of compound 4b.

### Synthesis Example 35-2

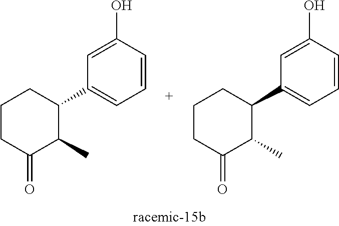

Similar to the synthesis of compound 15b from compound 4b, racemic compound 15b (0.55 g) was synthesized in a yield of 72% from racemic compound 4b (0.93 g, 3.75 mmol). The NMR spectrum of the resulting compound was consistent with that of compound 15b.

### Separation and Purification of Racemic or Less Optically Pure Compound 15b Using Preparative Chiral Column for Separation of Optically Active Compounds

Racemic compound 15b (290 mg) was loaded on a normal-phase preparative chiral column (CHIRALPAK AY-H, φ20 mm×250 mm, Daicel Corporation) for separation of optically active compounds and eluted using a mixed solvent of hexane and ethanol (90:10, v/v) as a mobile phase (5.7 mL/min). The eluate was continuously monitored with a UV absorption detector (254 nm) and fractionated. The eluted fractions were collected and concentrated to give optically active compound 15b (137 mg, Rt 7.8 min, 94% yield) and its enantiomer (2R, 3S) 15b (144 mg, Rt 10.2 min, 99% yield), respectively.

15b: [α]²⁰_{D}+14.6 (c 1.00, CHCl₃)

(2R,3S)15b: [α]²⁰′_{D}−15.0 (c 1.00, CHCl₃)

### Evaluation of Activity

### Test Example 1: Time-Resolved Fluorescence Resonance Energy Transfer (TR-FRET) Vitamin D Receptor (VDR) Coactivator Assay

A VDR ligand binding domain tagged with GST (VDR-LBD(GST)), a fluorescein-TRAP220/DRIP-2-bound coactivator peptide (Fluorescein-peptide), LanthaScreen Tb-anti-GST (Goat) antibody (Tb-anti-GST), TR-FRET co-regulator buffer G, and DTT solution were used from LanthaScreen TR-FRET VDR Coactivator Assay Kit, which was purchased from Invitrogen.

Each of the compounds synthesized as described above was dissolved in dimethyl sulfoxide (DMSO for molecular biology, Sigma Aldrich). The solution was diluted to a desired concentration with TR-FRET co-regulator buffer G containing 1 mass % of DMSO. The receptor-tracer-antibody complex solution was added to the compound solution in each well (20 μL) such that each well contained 1.0 nM of VDR-LBD(GST), 2.0 nM of Tb-anti-GST, and 100 nM of Fluorescein-peptide. The resulting mixture was incubated at room temperature for 2 hours.

Each well was measured for TR-FRET using a microplate reader (Infinite F200 PRO, Tecan) equipped with an excitation filter at 340 nm (30 nm bandwidth), a terbium emission filter at 495 nm (10 nm bandwidth), and a tracer emission filter at 520 nm (25 nm bandwidth). On the basis of the resulting data, the 50% effective concentration (EC₅₀) of each compound was calculated and evaluated using a graph plotting program (GraphPad Prism ver. 8.2.0) with the saturated activity of natural active vitamin D3 being normalized to 100%.

### Results

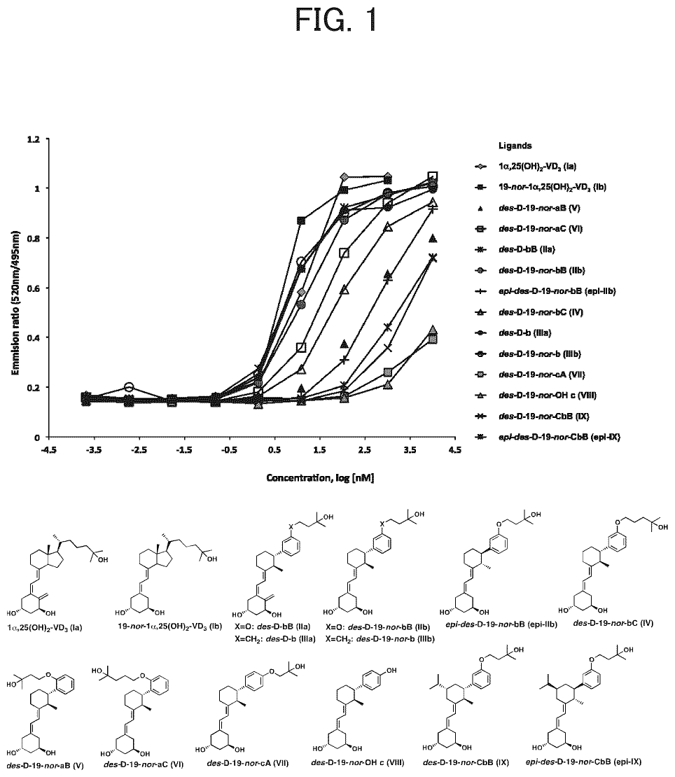

The time-resolved fluorescence resonance energy transfer (TR-FRET) vitamin D receptor (VDR) coactivator assay was used to assay the binding of the test compound to the vitamin D receptor, which is the first stage for its agonist activity, and the following formation of a complex between the coactivator protein and the receptor protein having undergone denaturation from apo- to holo-form. Compounds des-D-bB (IIa), des-D-b (IIIa), des-D-19-nor-bB (IIb), des-D-19-nor-b (IIIb), epi-des-D-19-nor-bB (epi-IIb), des-D-19-nor-bC (IV), des-D-19-nor-aB (V), des-D-19-nor-aC (VI), des-D-19-nor-cA (VII), des-D-19-nor-OH c (VIII), des-D-19-nor-CbB (IX), and epi-des-D-19-nor-CbB (epi-IX), which were synthesized according to the present invention, were assayed together with a natural active form of vitamin D3 (1α,25(OH)₂—VD₃ (Ia)) and a known active derivative (19-nor-1α,25(OH)₂—VD₃ (Ib)). The resulting concentration dependent curves for the fluorescence emission intensity ratio (Emission ratio) (an indicator of activity), in other words, for the ratio of the intensity of fluorescence emission at 520 nm to the intensity of fluorescence emission at 495 nm, are as shown in FIG. 1 (n=3), indicating that all of the derivatives assayed are effective in a concentration dependent manner. Table 1 shows a summary of the results, indicating that the half (50%) maximal effective concentration (EC₅₀) of the natural 1α,25(OH)₂—VD₃ (Ia) was 10.8 nM, 19-nor-1α,25(OH)₂-VD₃ (Ib) 4.8 nM, des-D-bB (IIa) 7.5 nM, des-D-b (IIIa) 7.4 nM, des-D-19-nor-bB (IIb) 17.4 nM, des-D-19-nor-b (IIIb) 6.7 nM, epi-des-D-19-nor-bB (epi-IIb) 518.4 nM, des-D-19-nor-bC (IV) 84.0 nM, des-D-19-nor-aB (V) 243.4 nM, des-D-19-nor-aC (VI) 50.3 nM, des-D-19-nor-cA (VII) >5000 nM, des-D-19-nor-OH c (VIII) >5000 nM, des-D-19-nor-CbB (IX) 922.0 nM, and epi-des-D-19-nor-CbB (epi-IX) 845.7 nM.

TABLE 1

| | | TR-FRET vitamin |
|---|---|---|
| | | D receptor |
| Compound name | Compound | coactivator assay: |
| abbreviation | No. | EC₅₀ (nM) |
| 1 α, 25 (OH)₂—VD₂ | Ia | 10.8 |
| 19-nor-1 α, 25 (OH)₂—VD₃ | Ib | 4.8 |
| des-D-bB | IIa | 7.5 |
| des-D-b | IIIa | 7.4 |
| des-D-19-nor-bB | IIb | 17.4 |
| des-D-19-nor-b | IIIb | 6.7 |
| epi-des-D-19-nor-bB | epi-IIb | 518.4 |
| des-D-19-nor-bC | IV | 84.0 |
| des-D-19-nor-aB | V | 243.4 |
| des-D-19-nor-aC | VI | 50.3 |
| des-D-19-nor-cA | VII | >5000 |
| des-D-19-nor-OH c | VIII | >5000 |
| des-D-19-nor-CbB | IX | 922.0 |
| epi-des-D-19-nor-CbB | epi-IX | 845.7 |

### Test Example 2: Vitamin D Receptor (NR1I1, VDR) Reporter Assay

VDR reporter cells (NR1I1, VDR) containing a VDR target gene and a corresponding luciferase gene, a cell recovery solution (CRM), a compound screening solution (CSM), calcitriol (1.0 mM in DMSO, standard agonist for VDR), a detection substrate, a detection buffer, and 96-well assay plates (white, sterile, collagen-coated) were used from Human Vitamin D Receptor (NR1I1, VDR) Reporter Assay System, which was purchased from INDIGO Biosciences, Inc.

Each of the compounds synthesized as described above was dissolved in dimethyl sulfoxide (DMSO for molecular biology, Sigma Aldrich). The solutions were each diluted with CSM to a desired concentration such that the resulting DMSO concentration was less than 0.4 mass % (hereinafter, the resulting solutions will be collectively referred to as the “VD3 solution”). The VDR reporter cell (NR1I1, VDR) solution was dispensed into assay plates (200 μL/well). The plates were incubated at 37° C. for 4 to 6 hours under conditions of at least 85% humidity and 5% CO₂.

After the incubation was completed, the solution on the assay plates was discarded, and 100 μL of the VD3 solution was dispensed into each well of the assay plates. The plates were incubated at 37° C. for 22 to 24 hours under conditions of at least 85% humidity and 5% CO₂. After the incubation was completed, the VD3 solution was discarded, and 100 μL of a luciferase detection reagent (LDR) prepared by mixing the detection substrate and the detection buffer was dispensed into each well of the assay plates. After the addition of the LDR, the assay plates were allowed to stand at room temperature for at least 5 minutes. A human vitamin D receptor reporter assay was performed in which the assay plates were measured using Infinite F200 PRO Microplate Reader (Tecan) in the luminescence mode. The EC₅₀ of each compound was calculated using GraphPad Prism (ver. 8.2.0).

### Results

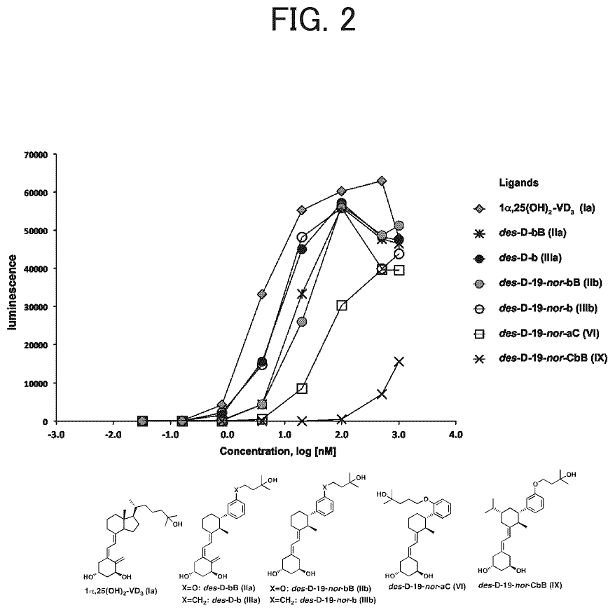

The vitamin D receptor reporter assay is performed to determine whether the test compound can induce the expression of the corresponding luciferase in the VDR reporter cells (NR1I1, VDR) containing the VDR target gene and the corresponding luciferase gene. The expression of the corresponding luciferase can occur if the test compound migrating into the cell nucleus can bind to the vitamin D receptor protein and then induce the binding of the coactivator protein to trigger the formation of a complex of various proteins necessary for the gene transcription so that the VDR target gene and the corresponding luciferase gene (the gene target sites) can be read out (transcribed). If the test compound is active in this assay, it can be shown to have an agonist activity. Compounds des-D-bB (IIa), des-D-b (IIIa), des-D-19-nor-bB (IIb), des-D-19-nor-b (IIIb), des-D-19-nor-aC (VI), and des-D-19-nor-CbB (IX), which were synthesized according to the present invention, were assayed together with a natural active form of vitamin D3 (1α,25(OH)₂—VD₃ (Ia)). The resulting concentration dependent curves for the luciferase fluorescence emission intensity (an indicator of activity) are as shown in FIG. 2 (n=3), indicating that all of the derivatives assayed are effective in a concentration dependent manner. Table 2 shows a summary of the results, indicating that the half (50%) maximal effective concentration (EC₅₀) of the natural 1α,25(OH)₂—VD₃ (Ia) was 3.4 nM, des-D-bB (IIa) 14.9 nM, des-D-b (IIIa) 8.2 nM, des-D-19-nor-bB (IIb) 22.5 nM, des-D-19-nor-b (IIIb) 7.5 nM, des-D-19-nor-aC (VI) 46.0 nM, and des-D-19-nor-CbB (IX) >500 nM.

TABLE 2

| Compound name | Compound | Vitamin D receptor (NR1I1, VDR) |
|---|---|---|
| abbreviation | No. | reporter assay: EC₅₀ (nM) |
| 1 α, 25 (OH)₂—VD₃ | Ia | 3.4 |
| des-D-bB | IIa | 14.9 |
| des-D-b | IIIa | 8.2 |
| des-D-19-nor-bB | IIb | 22.5 |
| des-D-19-nor-b | IIIb | 7.5 |
| des-D-19-nor-aC | VI | 46.0 |
| des-D-19-nor-CbB | IX | >500 |

1. A compound of Formula (1): 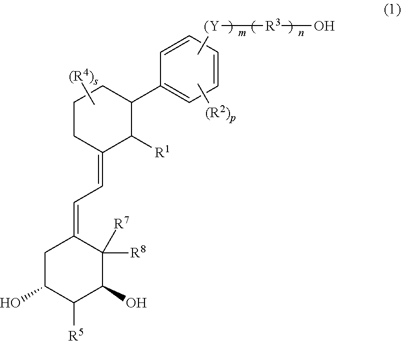
wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, R⁶ is a hydrogen atom or a monovalent aliphatic hydrocarbon group, and R⁷ and R⁸ are simultaneously hydrogen atoms or combined to form a methylidene group.

2. A prophylactic or therapeutic agent for a disease responsive to active vitamin D₃, the prophylactic or therapeutic agent comprising the compound according to claim 1 as an active ingredient.

3. The prophylactic or therapeutic agent according to claim 2, wherein the disease responsive to active vitamin D₃ is osteoporosis, rickets, chronic hypocalcemia, renal osteodystrophy, secondary hyperparathyroidism, psoriasis, or cancer.

4. A compound of Formula (2): 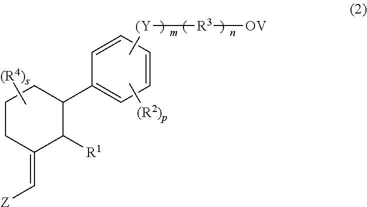
wherein m and n are independently 0 or 1, p is an integer of 0 to 4, s is an integer of 0 to 4, V is a hydrogen atom or a protecting group which is represented by W, Y is an oxygen atom, a sulfur atom, or a divalent group represented by —NR⁶—, Z is a halogen atom or a group represented by Formula (3) below, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, and R⁶ is a hydrogen atom or a monovalent aliphatic hydrocarbon group, 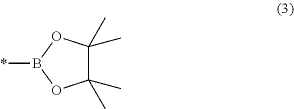 [SMILES: *B1OC(C)(C)C(C)(C)O1]
wherein * indicates a bond.

5. A compound of Formula (4): 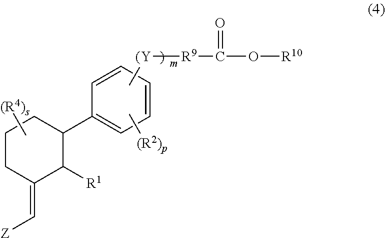
wherein m is 0 or 1, p is an integer of 0 to 4, s is an integer of 0 to 4, Y is an oxygen atom, a sulfur atom, or a divalent group represented by —NR⁶—, Z is a halogen atom or a group represented by Formula (3) below, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R⁴ is a linear or branched, saturated or unsaturated, monovalent aliphatic hydrocarbon group having 1 to 6 carbon atoms,
R⁶ is a hydrogen atom or a monovalent aliphatic hydrocarbon group, R⁹ is a linear or branched, saturated or unsaturated, divalent aliphatic hydrocarbon group having 1 to 19 carbon atoms, optionally having a substituent, and optionally having a heteroatom-containing carbon chain, and R¹⁰ is an alkyl group having 1 to 8 carbon atoms, 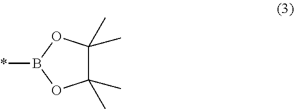 [SMILES: *B1OC(C)(C)C(C)(C)O1]
wherein * indicates a bond.

6. A compound of Formula (5): 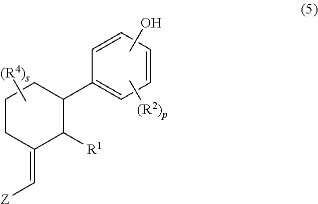
wherein p is an integer of 0 to 4, s is an integer of 0 to 6, Z is a halogen atom or a group represented by Formula (3) below, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, and R⁴ is a linear or branched, saturated or unsaturated, monovalent aliphatic hydrocarbon group having 1 to 6 carbon atoms, 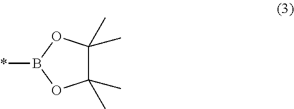 [SMILES: *B1OC(C)(C)C(C)(C)O1]
wherein * indicates a bond.

7. A compound of Formula (6): 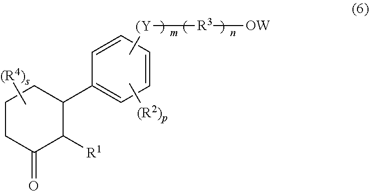
wherein m and n are independently 0 or 1, p is an integer of 0 to 4, s is an integer of 0 to 4, W is a protecting group, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, and R⁶ is a hydrogen atom or a monovalent aliphatic hydrocarbon group.

8. A method for producing a compound of Formula (1) below, the method comprising:
allowing a compound of Formula (7) below to react with a compound of Formula (8) below to form a compound of Formula (9) below; and
removing groups represented by W from the compound of Formula (9) to form the compound of Formula (1), 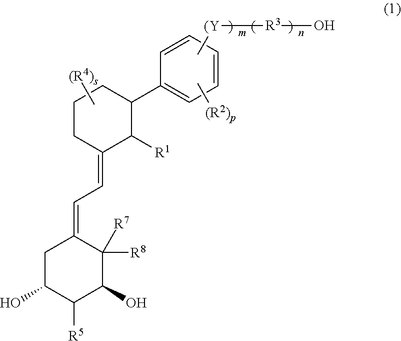
wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, R⁶ is a hydrogen atom or a monovalent aliphatic hydrocarbon group, and R⁷ and R⁸ are simultaneously hydrogen atoms or combined to form a methylidene group, 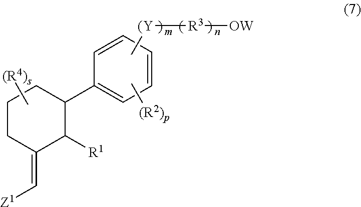
wherein m, n, p, s, Y, R¹, R², R³, R⁴, and R⁶ are as defined above, W is a protecting group, and Z¹ is a halogen atom or a group represented by Formula (3) below, 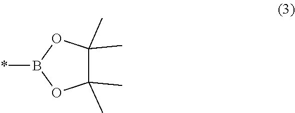
wherein * indicates a bond, 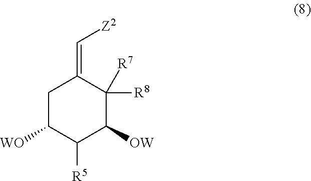
wherein R⁵, R⁷, R⁸, and W are as defined above, and Z² is a halogen atom or a group represented by Formula (3), provided that when Z¹ in Formula (7) is a halogen atom, Z² is a group represented by Formula (3) and that when Z¹ in Formula (7) is a group represented by Formula (3), Z² is a halogen atom, 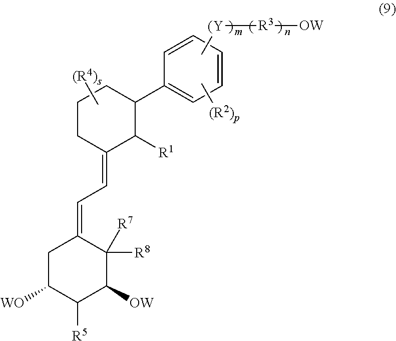
wherein m, n, p, s, W, Y, R¹, R², R³, R⁴, R⁵, R⁶, R⁷, and R⁸ are as defined above.

9. A method for producing a compound of Formula (13) below, the method comprising:
allowing a compound of Formula (10) below to react with a compound of Formula (8) below to form a compound of Formula (11) below;
obtaining a compound of Formula (12) below from the compound of Formula (11); and
removing groups represented by W from the compound of Formula (12) to form the compound of Formula (13), 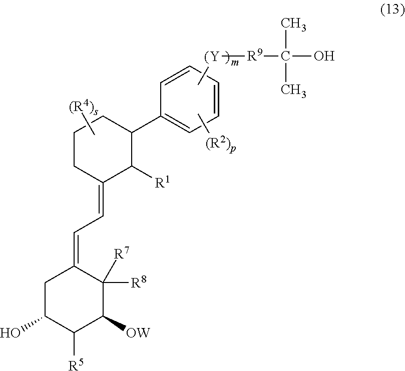
wherein m is 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, R⁶ is a hydrogen atom or a monovalent aliphatic hydrocarbon group, R⁷ and R⁸ are simultaneously hydrogen atoms or combined to form a methylidene group, and R⁹ is a linear or branched, saturated or unsaturated, divalent aliphatic hydrocarbon group having 1 to 19 carbon atoms, optionally having a substituent, and optionally having a heteroatom-containing carbon chain, 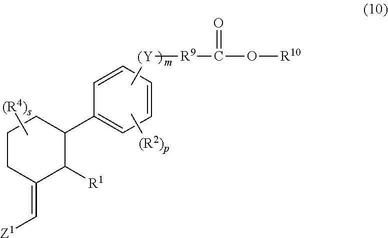
wherein m, p, s, Y, R¹, R², R⁴, R⁶, R⁹, and R¹⁰ are as defined above, R¹⁰ is an alkyl group having 1 to 8 carbon atoms, and Z¹ is a halogen atom or a group represented by Formula (3) below, 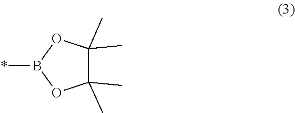
wherein * indicates a bond, 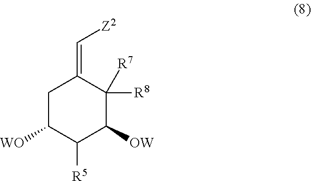
wherein R⁵, R⁷, and R⁸ are as defined above, W is a protecting group, and Z² is a halogen atom or a group represented by Formula (3), provided that when Z¹ in Formula (10) is a halogen atom, Z² is a group represented by Formula (3) and that when Z¹ in Formula (10) is a group represented by Formula (3), Z² is a halogen atom, 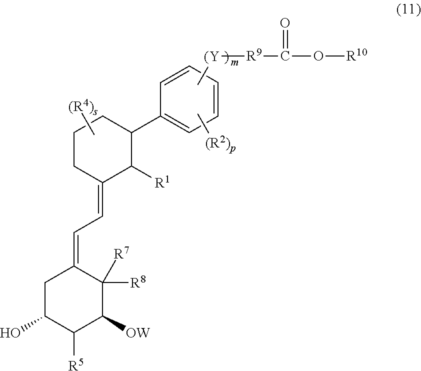
wherein m, p, s, W, Y, R¹, R², R⁴, R⁵, R⁶, R⁷, R⁸, R⁹, and R¹⁰ are as defined above, 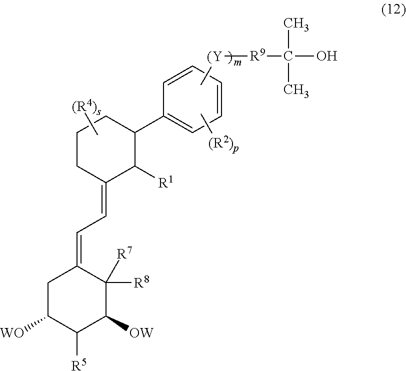
wherein m, p, s, W, Y, R¹, R², R⁴, R⁵, R⁶, R⁷, R⁸, and R⁹ are as defined above.

10. A method for producing a compound of Formula (16) below, the method comprising:
allowing a compound of Formula (14) below to react with a compound of Formula (8) below to form a compound of Formula (15) below; and
removing groups represented by W from the compound of Formula (15) to form the compound of Formula (16), 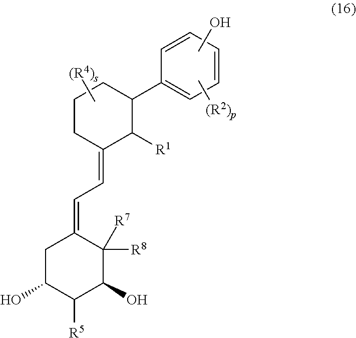
wherein p is an integer of 0 to 4, s is an integer of 0 to 4, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, and R⁷ and R⁸ are simultaneously hydrogen atoms or combined to form a methylidene group, 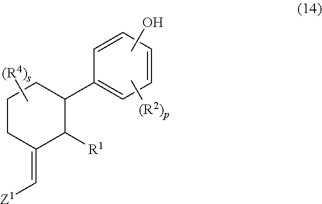
wherein p, s, R¹, R², and R⁴ are as defined above, and Z¹ is a halogen atom or a group represented by Formula (3) below, 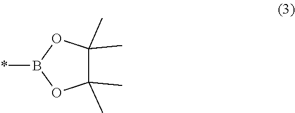
wherein * indicates a bond, 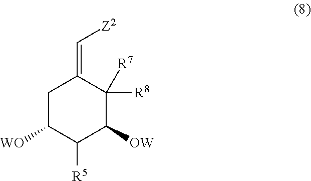
wherein R⁵, R⁷, and R⁸ are as defined above, W is a protecting group, and Z² is a halogen atom or a group represented by Formula (3), provided that when Z¹ in Formula (14) is a halogen atom, Z² is a group represented by Formula (3) and that when Z¹ in Formula (14) is a group represented by Formula (3), Z² is a halogen atom, 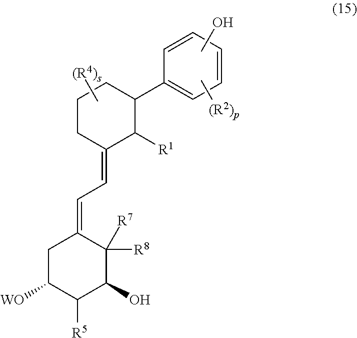
wherein p, s, W, R¹, R², R⁴, R⁵, R⁷, and R⁸ are as defined above.

11. A method for producing a compound of Formula (19) below, the method comprising:
allowing a compound of Formula (6) below to react with a compound of Formula (17) below to form a compound of Formula (18) below; and
removing groups represented by W from the compound of Formula (18) to form the compound of Formula (19), 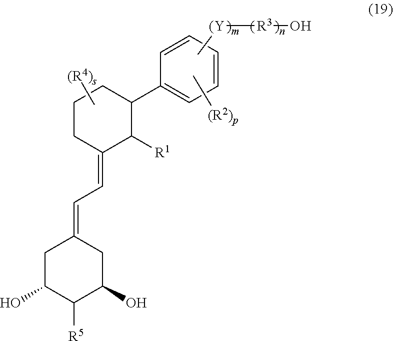
wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁵ is a hydrogen atom, a hydroxyl group, an alkoxy group having 1 to 6 carbon atoms and optionally having a substituent, or a linear or branched, saturated or unsaturated, monovalent aliphatic hydrocarbon group having 1 to 6 carbon atoms and optionally having a substituent, and R⁶ is a hydrogen atom or a monovalent aliphatic hydrocarbon group, 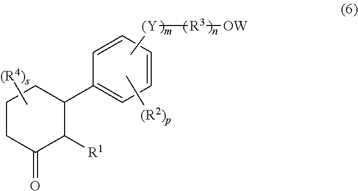
wherein m, n, p, s, Y, R¹, R², R³, R⁴, and R⁶ are as defined above, and W is a protecting group, 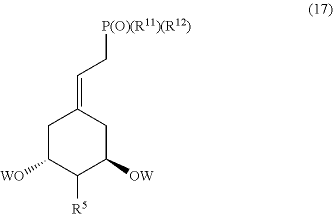
wherein R⁵ and W are as defined above, and R¹¹ and R¹² are independently a monovalent hydrocarbon group having 1 to 20 carbon atoms, 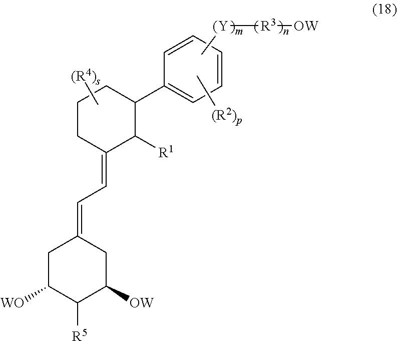
wherein m, n, p, s, W, Y, R¹, R², R³, R⁴, R⁵, and R⁶ are as defined above.

12. A method for producing a compound of Formula (23) below, the method comprising:
allowing a compound of Formula (7) below to react with a compound of Formula (20) below to form a compound of Formula (21) below;
obtaining a compound of Formula (22) below from the compound of Formula (21); and
removing a group represented by W from the compound of Formula (22) to form the compound of Formula (23), 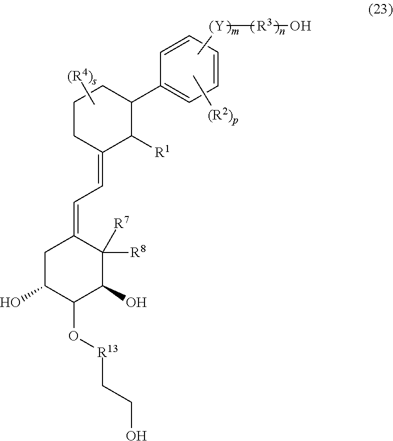
wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁶ is a hydrogen atom or a monovalent aliphatic hydrocarbon group, R⁷ and R⁸ are simultaneously hydrogen atoms or combined to form a methylidene group, and R¹³ is a linear or branched, saturated or unsaturated, divalent aliphatic hydrocarbon group having 1 to 4 carbon atoms, optionally having a substituent, and optionally having a heteroatom-containing carbon chain, 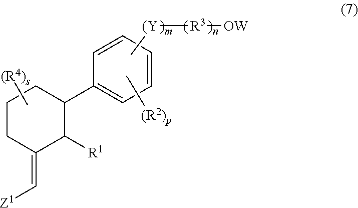
wherein m, n, p, s, Y, R¹, R², R³, R⁴, and R⁶ are as defined above, W is a protecting group, and Z¹ is a halogen atom or a group represented by Formula (3) below, 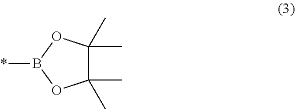 [SMILES: *B1OC(C)(C)C(C)(C)O1]
wherein * indicates a bond, 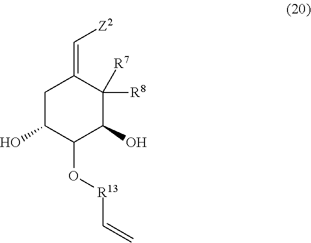
wherein R⁷, R⁸, and R¹³ are as defined above, and Z² is a halogen atom or a group represented by Formula (3), provided that when Z¹ in Formula (7) is a halogen atom, Z² is a group represented by Formula (3) and that when Z¹ in Formula (7) is a group represented by Formula (3), Z² is a halogen atom, 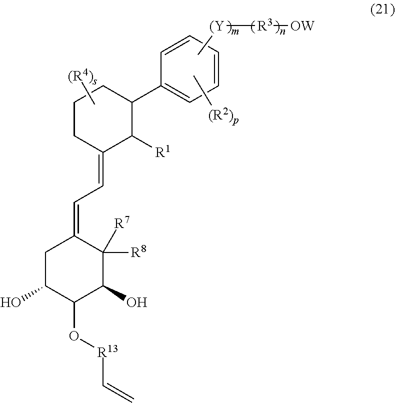
wherein m, n, p, s, Y, R¹, R², R³, R⁴, R⁶, R⁷, R⁸, and R¹³ are as defined above, and W is a protecting group, 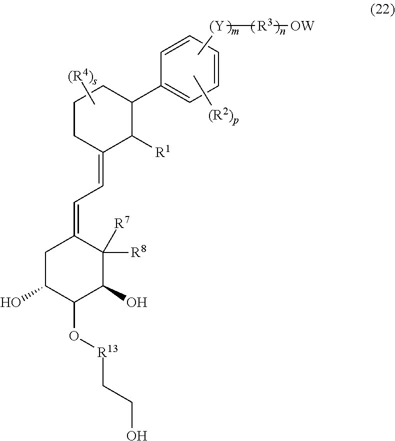
wherein m, n, p, s, Y, R¹, R², R³, R⁴, R⁶, R⁷, R⁸, R¹³, and W are as defined above.

13. A method for producing a compound of Formula (27) below, the method comprising:
allowing a compound of Formula (6) below to react with a compound of Formula (24) below to form a compound of Formula (25) below;
removing groups represented by W from the compound of Formula (25) to form a compound of Formula (26) below; and
obtaining the compound of Formula (27) from the compound of Formula (26), 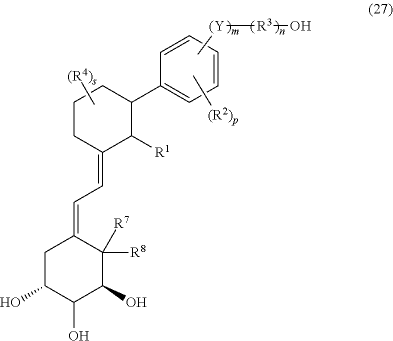
wherein m and n are independently 0 or 1, p is an integer of 0 to 4, s is an integer of 0 to 4, Y is an oxygen atom, a sulfur atom, or a divalent group represented by —NR⁶—, R¹ is a linear or branched, saturated or unsaturated, monovalent aliphatic hydrocarbon group having 1 to 6 carbon atoms, R² is an alkyl group having 1 to 8 carbon atoms, a hydroxyl group, an alkoxy group having 1 to 8 carbon atoms, or a halogen atom, R³ is a linear or branched, saturated or unsaturated, divalent aliphatic hydrocarbon group having 1 to 20 carbon atoms, optionally having a substituent, and optionally having a heteroatom-containing carbon chain, R⁴ is a linear or branched, saturated or unsaturated, monovalent aliphatic hydrocarbon group having 1 to 6 carbon atoms, R⁶ is a hydrogen atom or a monovalent aliphatic hydrocarbon group, and R⁷ and R⁸ are simultaneously hydrogen atoms or combined to form a methylidene group, 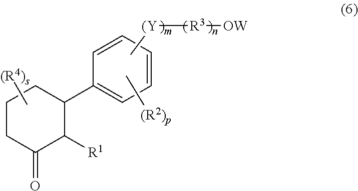
wherein m, n, p, s, Y, R¹, R², R³, R⁴, and R⁶ are as defined above, and W is a protecting group, 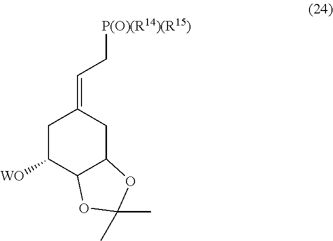
wherein W is as defined above, and R¹⁴ and R¹⁵ are independently a monovalent hydrocarbon group having 1 to 20 carbon atoms, 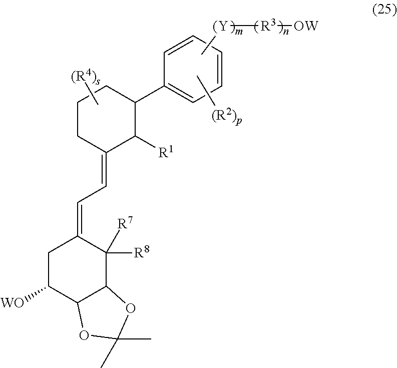
wherein m, n, p, s, Y, R¹, R², R³, R⁴, R⁶, R⁷, R⁸, and W are as defined above, 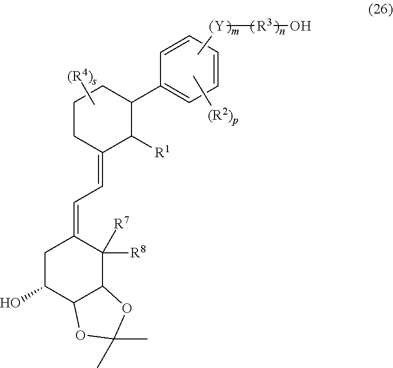
wherein m, n, p, s, Y, R¹, R², R³, R⁴, R⁶, R⁷, and R⁸ are as defined above.
